(19) United States
(12) Patent Application Publication
(10) Pub. No.: US 2008/0312301 A1
(43) Pub. Date: Dec. 18, 2008

(54) Substituted N-Benzo[D]Isoxazol-3-Yl-Amine Compounds as Inhibitors of Mglur5, Serotonin (5-Ht) and Noradrenaline Receptors, and Uses Thereof

(75) Inventors: Beatrix Merla, Aachen (DE); Matthias Gerlach, Brachttal (DE); Corinna Sundermann, Aachen (DE); Utz-Peter Jagusch, Aachen (DE); Hagen-Heinrich Hennies, Simmerath (DE); Stefan Oberborsch, Aachen (DE); Michael Haurand, Aachen (DE); Ruth Jostock, Stolberg (DE); Melanie Reich, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/916,613

(22) PCT Filed: Jun. 6, 2006
(86) PCT No.: PCT/EP06/05356

§ 371 Date: Jul. 2, 2008

(30) Foreign Application Priority Data
Jun. 6, 2005 (DE) 10 2005 026 194.9

(51) Int. Cl. A61K 31/423; A61P 25/00; A61P 1/00; C07D 261/20; C07D 413/12

(52) U.S. Cl. 514/379; 548/241

(57) ABSTRACT

The invention relates to substituted N-benzo[d]isoxazol-3-yl-amine derivatives of formula (I) wherein R¹, R², R³, R⁴ and R⁵ are defined as in patent claim 1. The invention also relates to the use of said compounds for producing medicaments.

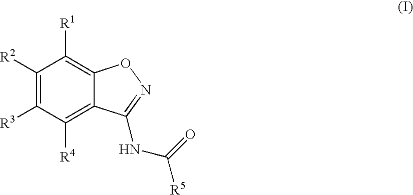

The present invention relates to substituted N-benzo[d]isoxazol-3-ylamine derivatives, process for their preparation, medicaments comprising these compounds, and the use of these compounds for producing medicaments.

Pain is one of the basic symptoms in clinical practice. There is a world-wide need for effective pain therapies. The pressing need for action for a treatment of chronic and non-chronic states of pain which is appropriate for patients and target-oriented, meaning by this the successful and satisfactory treatment of the patient's pain, is also demonstrated by the large number of scientific studies which have recently appeared in the field of applied analgesics and of basic research into nociception.

Classical opioids such as, for example, morphine are effective for the therapy of severe to very severe pain but often lead to unwanted side effects such as, for example, respiratory depression, vomiting, sedation, constipation or development of tolerance. They are moreover frequently insufficiently effective for neuropathic pain, from which tumor patients in particular suffer.

One object of the present invention was therefore to provide novel compounds which are suitable in particular as active pharmaceutical ingredients in medicaments, preferably in medicaments for the treatment of pain.

A further object of the present invention was to provide novel compounds which are suitable as pharmacological active ingredients in medicaments for the treatment of impairments or diseases which are mediated at least in part by mGluR5 receptors (mGluR5=metabotropic glutamate receptor 5) and/or serotonin (5-HT) receptors and/or noradrenaline receptors.

It has now been found, surprisingly, that substituted N-benzo[d]isoxazol-3-ylamine derivatives of the general formulae I and Ia indicated below have an excellent affinity for the mGluR5 receptor, for the serotonin (5-HT) receptor and for the noradrenaline receptor, and are therefore suitable in particular for the prophylaxis and/or treatment of disorders or diseases which are mediated at least partly by mGluR5 receptors (mGluR5=metabotropic glutamate receptor 5) and/or serotonin (5-HT) receptors and/or noradrenaline receptors.

The present invention therefore relates firstly to a medicament comprising at least one substituted N-benzo[d]isoxazol-3-ylamine derivative of the general formula I

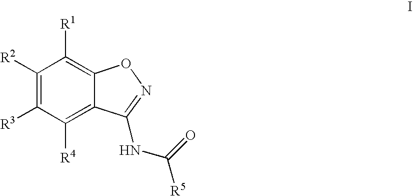

in which
R¹, R², R³ and R⁴ each independently of one another
   are H, F, Cl, Br, I, —CN, —NC, —NO₂, —SO₃H, —S(═O₂)NH₂, —NH₂, —OH, —SH, are a linear or branched C₁₋₁₀ alkyl radical, —OR⁶, —O—(CH₂)—R⁷, —SR⁸, —S—(CH₂)—R⁹, —NR¹⁰R¹¹, —NH—C(═O)—R¹², —NR¹³—C(═O)—R¹², —C(═O)—NH₂, —C(═O)—R¹⁴, —C(═O)—OH or —C(═O)—OR¹⁵;
R⁵ is a linear or branched, optionally substituted C₁₋₁₀ alkyl radical;
   is a linear or branched, optionally substituted C₂₋₁₀ alkenyl radical;
   is an unsaturated or saturated, optionally substituted 3-, 4-, 5-, 6-, 7-, 8- or 9-membered cycloaliphatic radical which may be bridged by 1 or 2 linear or branched C₁₋₅-alkylene groups;
   is an imidazolidinonyl radical which may be substituted by 1 or 2 substituents selected independently of one another from the group consisting of methyl, ethyl, n-propyl, isopropyl, n-butyl, isobutyl and tert-butyl;
   is an optionally substituted 6- to 10-membered aryl radical which may be fused to a saturated or unsaturated, optionally substituted mono- or polycyclic ring system;
   is a radical selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl, 4-methyl-3-nitrophenyl and 5-nitrofuranyl;
   is an optionally substituted 5- to 14-membered heteroaryl radical;
   is —(CH₂)ₙ—C(═O)—OR¹⁶ with n=0, 1, 2, 3, 4 or 5;
   is —CR¹⁷R¹⁸—O—C(═O)—R¹⁹;
   is —(CHR²⁰)—(CH₂)ₚ—R²¹ with p=0 or 1;
   or
   is —(CH═CH)—R²²;
R⁶, R⁸, R¹⁴ and R¹⁵ each independently of one another
   are a linear or branched, optionally substituted C₁₋₁₀ alkyl radical;
   are a linear or branched, optionally substituted C₂₋₁₀ alkenyl radical;
   or are an optionally substituted 5- to 14-membered aryl or heteroaryl radical;
R⁷ and R⁹ each independently of one another
   are an optionally substituted 5- to 14-membered aryl or heteroaryl radical;
R¹⁰ and R¹¹ each independently of one another
   are a hydrogen radical or
   are a linear or branched C₁₋₁₀ alkyl radical;
R¹², R¹³, R¹⁶ and R¹⁹ each independently of one another
   are a linear or branched C₁₋₁₀ alkyl radical;
R¹⁷ is a linear or branched C₁₋₁₀ alkyl radical
   or is an optionally substituted 6- to 10-membered aryl radical;
R¹⁸ and R²⁰ each independently of one another
   are a hydrogen radical;
   are a linear or branched C₁₋₁₀ alkyl radical;
   or
   are an optionally substituted 6- to 10-membered aryl radical;
R²¹ is an unsaturated or saturated, optionally substituted 3-, 4-, 5-, 6-, 7-, 8- or 9-membered cycloaliphatic radical,
   is an optionally substituted 6- to 10-membered aryl radical,
   or
   is an optionally substituted heteroaryl radical selected from the group consisting of thiophenyl, furanyl, benzo[b]furanyl and benzo[b]thiophenyl;
      and
R²² is an optionally substituted 6- to 10-membered aryl radical;
   in each case where appropriate in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates;
   and where appropriate one or more physiologically tolerated excipients.

The aforementioned optionally substituted C₁₋₁₀ alkyl radicals may preferably be unsubstituted or optionally each substituted by 1, 2, 3, 4, 5, 6, 7, 8 or 9 substituents independently of one another selected from the group consisting of F, Cl, Br, I, —CN, —OH, —SH and —NO₂.

The aforementioned optionally substituted C₂₋₁₀ alkenyl radicals can likewise preferably be unsubstituted or optionally each substituted by 1, 2, 3, 4, 5, 6, 7, 8 or 9 substituents independently of one another selected from the group consisting of F, Cl, Br, I, —CN, —OH, —SH and —NO₂.

Where one or more of the substituents R¹ to R⁴, R¹⁰ to R¹³ and R¹⁶ to R²⁰ are a linear or branched C₁₋₁₀-alkyl radical, this radical can preferably be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

If one or more of the substituents R⁵, R⁶, R⁸, R¹⁴ and R¹⁵ are a linear or branched, optionally substituted C₁₋₁₀-alkyl radical, this radical can preferably be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl and optionally be substituted by 1, 2, 3, 4, 5, 6, 7, 8 or 9 substituents independently of one another selected from the group consisting of F, Cl, Br, I, —CN and —NO₂.

Particularly preferred optionally substituted C₁₋₁₀-alkyl radicals can be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃ and —CH₂—CH₂—CH₂—CN.

If one or more of the substituents R⁵, R⁶, R⁸, R¹⁴ and R¹⁵ are a linear or branched optionally substituted C₂₋₁₀-alkenyl radical, this radical can preferably be selected from the group consisting of 1-propenyl, 2-propenyl, 1-butenyl, 2-butenyl, 3-butenyl and 2-methyl-1-propenyl and optionally be substituted by 1, 2, 3, 4, 5, 6, 7, 8 or 9 substituents independently of one another selected from the group consisting of F, Cl, Br, I, —CN and —NO₂.

The aforementioned optionally substituted cycloaliphatic radicals can preferably be unsubstituted or optionally each substituted by 1, 2, 3, 4 or 5 substituents independently of one another selected from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—H, —C(═O)—C₁₋₅-alkyl, —C(═O)—O—C₁₋₅-alkyl, —(CH₂)—C(═O)—O—C₁₋₅-alkyl, —NH—C₁₋₅-alkyl, —N(C₁₋₅-alkyl)₂, —(CH₂)-benzo[b]furanyl, —O-phenyl, —O-benzyl, phenyl, benzyl, naphthyl and —(CH₂)-naphthyl, where in each case the cyclic part of the radicals —O-phenyl, —O-benzyl, phenyl, —(CH₂)-benzo[b]furanyl, benzyl, naphthyl and —(CH₂)-naphthyl may be substituted by 1, 2, 3, 4 or 5 substituents independently of one another selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl.

Where the substituent R⁵ and/or R²¹ is a cycloaliphatic radical which may optionally be bridged by 1 or 2 linear or branched C₁₋₅-alkylene groups, this can preferably be selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantyl.

The cycloaliphatic radicals can particularly preferably each be optionally substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂, —N(CH(CH₃)₂)₂, —(CH₂)-benzo[b]furanyl, —O-phenyl, —O-benzyl, phenyl, benzyl, naphthyl and —(CH₂)-naphthyl, where in each case the cyclic part of the radicals —O-phenyl, —O-benzyl, phenyl, —(CH₂)-benzo[b]furanyl, benzyl, naphthyl and —(CH₂)-naphthyl can be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇, methyl, ethyl, n-propyl, isopropyl, n-butyl, sec-butyl, isobutyl, tert-butyl, n-pentyl, 2-pentyl, 3-pentyl, neopentyl, —O—CF₃, —S—CF₃, phenyl and —O-benzyl.

The aforementioned optionally substituted aryl or heteroaryl radicals may likewise preferably be unsubstituted or optionally each substituted by 1, 2, 3, 4 or 5 substituents independently of one another selected from the group consisting of F, Cl, Br, I, —CN, —CF₃, —SF₅, —OH, —O—C₁₋₅-alkyl, —O—C₂₋₅-alkenyl, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl may be substituted by 1, 2, 3, 4 or 5 substituents independently of one another selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl.

The aforementioned heteroaryl radicals may preferably optionally each have 1, 2, 3, 4 or 5 heteroatom(s) independently of one another selected from the group consisting of oxygen, nitrogen and sulfur as ring member(s).

Where one or more of the substituents R⁵ to R⁹, R¹⁴, R¹⁵, R¹⁷, R¹⁸ and R²⁰ to R²² are an aryl radical, this can preferably be selected from the group consisting of phenyl and naphthyl (1-naphthyl and 2-naphthyl).

Where one or more of the substituents R⁵ to R⁹, R¹⁴, R¹⁵ and R²¹ are a heteroaryl radical, this can preferably be selected from the group consisting of thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl.

The aryl or heteroaryl radicals can particularly preferably optionally each be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CH₂—CH═CH₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —C(═O)—H, —C(═O)—CH₃, —C(═O)—C₂H₅, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, —(CH₂)-benzo[b]furanyl, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇, methyl, ethyl, n-propyl, isopropyl, n-butyl, sec-butyl, isobutyl, tert-butyl, n-pentyl, 2-pentyl, 3-pentyl, neopentyl, —O—CF₃, —S—CF₃, phenyl and —O-benzyl.

The rings of the aforementioned optionally substituted mono- or polycyclic ring systems may likewise preferably be unsubstituted or optionally each substituted by 1, 2, 3, 4 or 5 substituents independently of one another selected from the group consisting of oxo (═O), thioxo (═S), F, Cl, Br, I, —CN, —CF₃, —SF₅, —OH, —O—C₁₋₅-alkyl, —O—C₂₋₅-alkenyl, —NH₂, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl and —S(═O)₂—C₁₋₅-alkyl.

The rings of the aforementioned optionally substituted mono- or polycyclic ring systems may particularly preferably be unsubstituted or optionally each substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —C(═O)—H, —C(═O)—CH₃, —C(═O)—C₂H₅, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅ and —S(═O)₂—CH(CH₃)₂.

A mono- or polycyclic ring system means in the context of the present invention mono- or polycyclic hydrocarbon radicals which may be saturated or unsaturated and optionally have 1, 2, 3, 4 or 5 heteroatom(s) as ring member(s) which are selected independently of one another from the group consisting of oxygen, nitrogen and sulfur.

Such a mono- or polycyclic ring system may for example be fused to an aryl radical or a heteroaryl radical.

If a polycyclic ring system such as, for example, a bicyclic ring system is present, the various rings may each independently of one another have a different degree of saturation, i.e. be saturated or unsaturated. A polycyclic ring system is preferably a bicyclic ring system.

The rings of the aforementioned mono- or polycyclic ring systems preferably each have 5, 6 or 7 members and may each have 1, 2 or 3 heteroatom(s) as ring member(s) which are selected independently of one another from the group consisting of oxygen, nitrogen and sulfur.

Examples which may be mentioned of aryl radicals which may be fused to a mono- or polycyclic ring system are [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl.

Where the radical R⁵ has a linear or branched C₁₋₅-alkylene group, this can preferably be selected from the group consisting of —(CH₂)—, —(CH₂)₂—, —C(H)(CH₃)—, —C(CH₃)₂—, —(CH₂)₃—, —(CH₂)₄—, —(CH₂)₅—, —C(H)(C(H)(CH₃)₂)— and —C(C₂H₅)(H)—.

Preferred medicaments comprise at least one compound of the general formula I indicated above, in which

R⁶, R⁸, R¹⁴ and R¹⁵ independently of one another each
are a radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃, —CH₂—CH₂—CH₂—CN, 1-propenyl, 2-propenyl, 1-butenyl, 2-butenyl, 3-butenyl and 2-methyl-1-propenyl;
or are a radical selected from the group consisting of phenyl, naphthyl, thiophenyl, furanyl and pyridinyl, where the radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
and in each case R¹ to R⁵, R⁷, R⁹ to R¹³ and R¹⁶ to R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred medicaments comprise at least one compound of the general formula I indicated above, in which

R⁷ and R⁹ independently of one another each are
a radical selected from the group consisting of phenyl, naphthyl, thiophenyl, furanyl and pyridinyl, where the radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
and in each case R¹ to R⁶, R⁸ and R¹⁰ to R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred medicaments comprise at least one compound of the general formula I indicated above, in which

R¹⁰ and R¹¹ independently of one another each are a hydrogen radical
or are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl;
and in each case R¹ to R⁹ and R¹² to R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred medicaments comprise at least one compound of the general formula I indicated above, in which

R¹², R¹³, R¹⁶ and R¹⁹ independently of one another each are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl;
and in each case R¹ to R¹¹, R¹⁴, R¹⁵, R¹⁷, R¹⁸ and R²⁰ to R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred medicaments comprise at least one compound of the general formula I indicated above, in which
R¹⁷ is an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl,
   or is a phenyl or naphthyl radical, where the radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and in each case R¹ to R¹⁶ and R¹⁸ to R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred medicaments comprise at least one compound of the general formula I indicated above, in which
R¹⁸ and R²⁰ independently of one another each
   are a hydrogen radical;
   are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl, or are a phenyl, or naphthyl radical, where the radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and in each case R¹ to R¹⁷, R¹⁹, R²¹ and R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred medicaments comprise at least one compound of the general formula I indicated above, in which
R²¹ is a cycloaliphatic radical selected from the group consisting of cyclopropyl, cyclobutyl, cyclopentyl, cyclohexyl, cycloheptyl, cyclopentenyl, cyclohexenyl and cycloheptenyl, where the cycloaliphatic radical may in each case optionally be substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂ and —N(CH(CH₃)₂)₂;
   or is a phenyl radical, where the phenyl radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and in each case R¹ to R²⁰ and R²² have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred medicaments comprise at least one compound of the general formula I indicated above, in which
R²² is a phenyl radical, where the phenyl radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.
   and in each case R¹ to R²¹ have the aforementioned meaning, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Particularly preferred medicaments comprise at least one compound of the general formula I indicated above, characterized in that
R¹, R², R³ and R⁴ independently of one another each
   are H, F, Cl, Br, I, —CN, —NC, —NO₂, —SO₃H, —S(═O₂)NH₂, —NH₂, —OH, —SH, methyl, ethyl, n-propyl, isopropyl, n-butyl, sec-butyl, isobutyl, tert-butyl, n-pentyl, sec-pentyl, n-hexyl, —OR⁶, —O—(CH₂)—R⁷, —SR⁸, —S—(CH₂)—R⁹, —NR¹⁰R¹¹, —NH—C(═O)—R¹², —NR¹³—C(═O)—R¹², —C(═O)—NH₂, —C(═O)—R¹⁴, —C(═O)—OH or —C(═O)—OR¹⁵;
R⁵ is a radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃, —CH₂—CH₂—CH₂—CN, 1-propenyl, 2-propenyl, 1-butenyl, 2-butenyl, 3-butenyl and 2-methyl-1-propenyl;
   is a cycloaliphatic radical selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantyl, where the cycloaliphatic radical may in each case optionally be substituted by 1, 2, 3, 4 or 5 substituents independently of one another selected from the group consisting of oxo (═O), thioxo (═S), F, Cl, Br, I, —CN, —CF₃, —SF₅, —O—CH₃, —O—C₂H₅, —O—C₃H₇, methyl, ethyl, n-propyl, isopropyl, n-butyl, sec-butyl, isobutyl, tert-butyl, phenyl and benzyl, where in each case the cyclic part of the radicals phenyl and benzyl may be substituted by 1, 2, 3, 4 or 5 substituents independently of one another selected from the group consisting of F, Cl, Br, methyl, ethyl and n-propyl;
   is an imidazolidinonyl radical;
   is a phenyl or naphthyl radical, where the radical may in each case be substituted by 1, 2, 3, 4 or 5 substituents independently of one another selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, phenyl and benzyl, where in each case the cyclic part of the radicals phenyl and benzyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇ methyl, ethyl, —O—CF₃ and —S—CF₃;
   is a radical selected from the group consisting of [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl, where the radical may in each case be substituted by 1, 2, 3, 4 or 5 substituents independently of one another selected from the group consisting of methyl, ethyl, n-propyl, isopropyl, n-butyl, sec-butyl, isobutyl and tert-butyl;
   is a radical selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl, 4-methyl-3-nitrophenyl and 5-nitrofuranyl;
   is a heteroaryl radical selected from the group consisting of thiophenyl, furanyl, pyrazolyl, pyrazinyl, pyranyl, triazolyl, pyridinyl, indolyl, benzo[b]furanyl, benzo[b]thiophenyl, thiazolyl, oxazolyl, isoxazolyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl, where the heteroaryl radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CH₂—CH═CH₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, dihydro[b]benzofuranyl, pyridinyl and benzyl may be substituted by 1, 2, 3, 4 or 5 substituents independently of one another selected from the group consisting of F, Cl, Br, —CF₃, —CN, —NO₂, —O—CH₃, —O—C₂H₅, —O—C₃H₇, methyl, ethyl, n-propyl, isopropyl, n-butyl, sec-butyl, isobutyl, tert-butyl, —O—CF₃ and —S—CF₃;
   is —(CH₂)ₙ—C(═O)—OR¹⁶ with n=0, 1, 2, 3 or 4;
   is —CR¹⁷R¹⁸—O—C(═O)—R¹⁹;
   is —(CHR²⁰)—(CH₂)ₚ—R²¹ with p=0 or 1;
   or
   is —(CH═CH)—R²²;
R⁶, R⁸, R¹⁴ and R¹⁵ independently of one another each
   are a radical selected from the group consisting of methyl, ethyl, n-propyl, isopropyl, n-butyl, isobutyl, tert-butyl, —CF₃, —CFH₂, —CF₂H, —CF₂—CF₃, —CH₂—CF₃ and —CF₂—CF₂—CF₃;
R⁷ and R⁹ independently of one another each are
   a phenyl radical which may be substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
R¹⁰ and R¹¹ independently of one another each
   are an alkyl radical selected from the group consisting of methyl, ethyl, n-propyl, isopropyl, n-butyl, isobutyl and tert-butyl;
R¹², R¹³, R¹⁶ and R¹⁹ independently of one another each
   are an alkyl radical selected from the group consisting of methyl, ethyl, n-propyl, isopropyl and tert-butyl;
R¹⁷ is an alkyl radical selected from the group consisting of methyl, ethyl, n-propyl, isopropyl and n-butyl,
   or is a phenyl radical;
R¹⁸ is a hydrogen radical,
   or is an alkyl radical selected from the group consisting of methyl, ethyl, n-propyl, isopropyl, n-butyl, isobutyl and tert-butyl,
R²⁰ is a hydrogen radical;
   is an alkyl radical selected from the group consisting of methyl, ethyl, n-propyl, isopropyl, n-butyl, isobutyl and tert-butyl,
   or is a phenyl radical;
R²¹ is a cycloaliphatic radical selected from the group consisting of cyclopropyl, cyclobutyl, cyclopentyl, cyclohexyl, cycloheptyl, cyclopentenyl, cyclohexenyl and cycloheptenyl;
   is a phenyl radical which may be substituted by 1, 2, 3, 4 or 5 substituents independently of one another selected from the group consisting of F, Cl, Br, I, —CN, —CF₃, —SF₅, —O—CH₃, —O—C₂H₅, —O—C₃H₇, —O—CF₃, —O—CHF₂, —O—CH₂F, methyl, ethyl, n-propyl, isopropyl, n-butyl, sec-butyl, isobutyl and tert-butyl;
   or is a thiophenyl radical;
      and
is a phenyl radical which may be substituted by 1, 2, 3, 4 or 5 substituents independently of one another selected from the group consisting of F, Cl, Br, and —CF₃.

Very particularly preferred medicaments comprise at least one compound of the general formula I indicated above, characterized in that
R¹ is H, F, Cl or Br;
R² is H, F, Cl, Br or —NH₂;
R³ is H, F, Cl, Br, methyl, ethyl, n-propyl, isopropyl, n-butyl, isobutyl and tert-butyl;
R⁴ is H, F, Cl, Br, —NH₂, methyl, ethyl, n-propyl, isopropyl, n-butyl, isobutyl and tert-butyl, —OR⁶, —O—(CH₂)—R⁷ or —NR¹⁰R¹¹;
R⁵ is a radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CF₂—CF₃, —CH₂—CF₃, —CF₂—CF₂—CF₃, —CH₂—CH₂—CF₃ and —CF₂—CF₂—CF₂—CF₃;
   is a cycloaliphatic radical selected from the group consisting of cyclopropyl, cyclobutyl, cyclopentyl, cyclohexyl, cycloheptyl, cyclopentenyl, cyclohexenyl, cycloheptenyl, (6,6)-dimethyl-[3.1.1]-bicycloheptyl and adamantyl;
   is a radical selected from the group consisting of phenyl, 2-trifluoromethylphenyl, 2-nitrophenyl, 2-dimethylaminophenyl, 2-diethylaminophenyl, 2-aminophenyl, 2-ethylphenyl, 2-methylbenzoate, 2-bromophenyl, 2-chlorophenyl, 2-fluorophenyl, 2-methylphenyl, 2-methoxyphenyl, 2-ethoxyphenyl, 2-cyanophenyl, 2-acetylphenyl, 2-dimethylaminosulfonylphenyl, 3-chlorophenyl, 3-methylphenyl, 3-nitrophenyl, 3-trifluoromethylphenyl, 3-fluorophenyl, 3-bromophenyl, 3-dimethylaminophenyl, 3-diethylaminophenyl, 3-aminophenyl, 3-methoxyphenyl, 3-ethylphenyl, 3-ethoxyphenyl, 3-cyanophenyl, 3-acetylphenyl, 3-phenylphenyl, 3-dimethylaminosulfonylphenyl, 4-bromophenyl, 4-methoxyphenyl, 4-chlorophenyl, 4-fluorophenyl, 4-tert-butylphenyl, 4-cyanophenyl, 4-nitrophenyl, 4-methylphenyl, 4-phenylphenyl, 4-trifluoromethylphenyl, 4-dimethylaminophenyl, 4-diethylaminophenyl, 4-aminophenyl, 4-iodophenyl, 4-n-propylphenyl, 4-di-n-propylaminosulfonylphenyl, 4-diethyl-aminosulfonylphenyl, 4-dimethylaminosulfonylphenyl, 4-ethylphenyl, 4-ethoxyphenyl, 4-methylbenzoate, 4-acetylphenyl, 2-fluoro-3-trifluoromethylphenyl, (2,3)-difluorophenyl, (2,3)-dimethylphenyl, (2,3)-dichlorophenyl, 3-fluoro-2-trifluoromethylphenyl, (2,4)-dichlorophenyl, (2,4)-difluorophenyl, 4-fluoro-2-trifluoromethylphenyl, (2,4)-dimethoxyphenyl, 2-chloro-4-fluorophenyl, (2,4)-dibromophenyl, 2-fluoro-4-trifluoromethylphenyl, (2,5)-difluorophenyl, 2-fluoro-5-trifluoromethylphenyl, 5-fluoro-2-trifluoromethylphenyl, 5-chloro-2-trifluoro-methylphenyl, 5-bromo-2-trifluoromethylphenyl, (2,5)-dimethoxyphenyl, (2,5)-bistrifluoromethylphenyl, (2,5)-dichlorophenyl, (2,5)-dibromophenyl, 2-fluoro-6-trifluoromethylphenyl, (2,6)-dimethoxyphenyl, (2,6)-dimethylphenyl, 2-chloro-6-fluorophenyl, 2-bromo-6-chlorophenyl, 2-bromo-6-fluorophenyl, (2,6)-difluorophenyl, (2,6)-dibromophenyl, (2,6)-dichlorophenyl, (3,4)-dichlorophenyl, 4-chloro-3-nitrophenyl, (3,4)-dimethoxyphenyl, 4-fluoro-3-trifluoromethylphenyl, 3-fluoro-4-trifluoromethylphenyl, (3,4)-difluorophenyl, 4-bromo-3-methylphenyl, 4-bromo-5-methylphenyl, 3-chloro-4-fluorophenyl, 4-fluoro-3-nitrophenyl, 4-bromo-3-nitrophenyl, (3,4)-dibromophenyl, 4-chloro-3-methylphenyl, 4-fluoro-3-methylphenyl, 4-methyl-3-nitrophenyl, (3,5)-dimethoxyphenyl, (3,5)-bis-trifluoromethylphenyl, (3,5)-difluorophenyl, (3,5)-dinitrophenyl, (3,5)-dichlorophenyl, 3-fluoro-5-trifluoromethylphenyl, 5-fluoro-3-trifluoromethylphenyl, (3,5)-dibromophenyl, 5-chloro-4-fluorophenyl, 5-chloro-4-fluorophenyl, 5-bromo-4-methylphenyl, (2,3,4)-trifluorophenyl, (2,3,4)-trichlorophenyl, (2,3,6)-trifluorophenyl, (2,4,6)-trichlorophenyl, (2,4,5)-trifluorophenyl, (2,4,5)-trichlorophenyl, (2,4)-dichloro-5-fluorophenyl, (2,4,6)-trichlorophenyl, (2,4,6)-trimethylphenyl, (2,4,6)-trifluorophenyl, (2,4,6)-trimethoxyphenyl, (3,4,5)-trimethoxyphenyl, (2,3,4,5)-tetrafluorophenyl and (2,3,4,5,6)-pentafluorophenyl;
   is a naphthyl radical;
   is a heteroaryl radical selected from the group consisting of thiophenyl, furanyl, pyridinyl, benzo[b]furanyl, benzo[b]thiophenyl, oxazolyl and isoxazolyl, where the heteroaryl radical may in each case optionally be substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
   is —(CH₂)ₙ—C(═O)—OR¹⁶ with n=1 or 2,
   or
   is —(CHR²⁰)—(CH₂)ₚ—R²¹ with p=0 or 1;
R⁶ is a radical selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R⁷ is a phenyl radical which may be substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
R¹⁰ and R¹¹ independently of one another each are an alkyl radical selected from the group consisting of methyl, ethyl, n-propyl, isopropyl, n-butyl, isobutyl and tert-butyl;
R¹⁶ is an alkyl radical selected from the group consisting of methyl, ethyl, n-propyl, isopropyl, n-butyl, isobutyl and tert-butyl;
R²⁰ is a hydrogen radical;
   is an alkyl radical selected from the group consisting of methyl, ethyl, n-propyl, isopropyl, n-butyl, isobutyl and tert-butyl,
   or is a phenyl radical, and
R²¹ is a cycloaliphatic radical selected from the group consisting of cyclopropyl, cyclobutyl, cyclopentyl, cyclohexyl, cycloheptyl, cyclopentenyl, cyclohexenyl and cycloheptenyl;
   is a phenyl radical which may be substituted by 1, 2, 3, 4 or 5 substituents independently of one another selected from the group consisting of F, Cl, Br, I, —CN, —CF₃, —SF₅, —O—CH₃, —O—C₂H₅, —O—C₃H₇, —O—CF₃, —O—CHF₂, —O—CH₂F, methyl, ethyl, n-propyl, isopropyl, n-butyl, sec-butyl, isobutyl and tert-butyl;
   or is a thiophenyl radical;
      in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of corresponding salts, or in each case in the form of corresponding solvates.

Yet further preferred medicaments comprise at least one compound of the general formula I selected from the group consisting of
[1] thiophene-2-benzo[d]isoxazol-3-ylcarboxamide,
[2] N-benzo[d]isoxazol-3-ylsuccinic acid methyl ester
[3] naphthalene-2-benzo[d]isoxazol-3-ylcarboxamide,
[4] adamantane-2-benzo[d]isoxazol-3-ylcarboxamide,
[5] cyclohexane-benzo[d]isoxazol-3-ylcarboxamide,
[6] N-benzo[d]isoxazol-3-yl-2,2-dimethylpropionamide,
[7] N-(4-methoxybenzo[d]isoxazol-3-yl)-2,2-diphenylacetamide,
[8] cyclopropane(5-bromobenzo[d]isoxazol-3-yl)carboxamide,
[9] 3,5-dichloro-N-(6-fluorobenzo[d]isoxazol-3-yl)benzamide,
[10] N-(4-aminobenzo[d]isoxazol-3-yl)-4-nitrobenzamide,
[11] naphthalene-1-(4-aminobenzo[d]isoxazol-3-yl)carboxamide,
[12] benzo[b]thiophene-2-(4-fluorobenzo[d]isoxazol-3-yl)carboxamide,
[13] N-benzo[d]isoxazol-3-yl-2-trifluoromethyl benzamide,
[14] N-benzo[d]isoxazol-3-yl-3,4-dichlorobenzamide
[15] N-benzo[d]isoxazol-3-yl-2,3-difluorobenzamide,
[16] N-(4-dimethylaminobenzo[d]isoxazol-3-yl)-3-fluorobenzamide,
[17] thiophene-2-(6-fluorobenzo[d]isoxazol-3-yl)carboxamide,
[18] N-(6-fluorobenzo[d]isoxazol-3-yl)-2-methylbutyramide,
[19] N-(6-chlorobenzo[d]isoxazol-3-yl)-2-fluoro-3-trifluoromethylbenzamide,
[20] N-(6-chlorobenzo[d]isoxazol-3-yl)-3-methoxybenzamide,
[21] N-(6-bromobenzo[d]isoxazol-3-yl)-4-tert-butylbenzamide,
[22] N-(6-bromobenzo[d]isoxazol-3-yl)-2-methoxybenzamide,
[23] N-(6-bromobenzo[d]isoxazol-3-yl)-2-trifluoromethylbenzamide,
[24] adamantane-2-(6-bromobenzo[d]isoxazol-3-yl)carboxamide,
[25] N-(6-bromobenzo[d]isoxazol-3-yl)-2-methylbutyramide,
[26] N-(5-fluorobenzo[d]isoxazol-3-yl)-2-trifluoromethylbenzamide,
[27] 3,4-dichloro-N-(5-fluorobenzo[d]isoxazol-3-yl)benzamide,
[28] N-(5-fluorobenzo[d]isoxazol-3-yl)-2-methylbenzamide,
[29] N-(5-bromobenzo[d]isoxazol-3-yl)-2-fluoro-5-trifluoromethylbenzamide,
[30] N-(5-bromobenzo[d]isoxazol-3-yl)-2,4-difluorobenzamide,
[31] N-(5-methylbenzo[d]isoxazol-3-yl)-2-trifluoromethyl benzamide,
[32] 3-fluoro-N-(5-methylbenzo[d]isoxazol-3-yl)benzamide,
[33] N-(4-methoxybenzo[d]isoxazol-3-yl)-3,5-bis-trifluoromethylbenzamide,
[34] 3,5-difluoro-N-(4-methoxybenzo[d]isoxazol-3-yl)benzamide,
[35] N-(4-dimethylaminobenzo[d]isoxazol-3-yl)-3,5-bis-trifluoromethylbenzamide,
[36] 2-bromo-N-[4-(2,2,2-trifluoroethoxy)benzo[d]isoxazol-3-yl]benzamide,
[37] 3-chloro-N-[4-(2,2,2-trifluoroethoxy)benzo[d]isoxazol-3-yl]benzamide,
[38] 4-tert-butyl-N-[4-(2,2,2-trifluoroethoxy)benzo[d]isoxazol-3-yl]benzamide,
[39] 2-methoxy-N-[4-(2,2,2-trifluoroethoxy)benzo[d]isoxazol-3-yl]benzamide,
[40] N-(6-chlorobenzo[d]isoxazol-3-yl)-4-iodobenzamide,
[41] naphthalene-2-(6-chlorobenzo[d]isoxazol-3-yl)carboxamide,
[42] N-(6-chlorobenzo[d]isoxazol-3-yl)-3,4-difluorobenzamide,
[43] N-(6-bromobenzo[d]isoxazol-3-yl)-4-fluoro-2-trifluoromethylbenzamide,
[44] 2,4-dichloro-N-(6-bromobenzo[d]isoxazol-3-yl)-5-fluorobenzamide,
[45] N-(6-bromobenzo[d]isoxazol-3-yl)-3-fluoro-4-trifluoromethyl benzamide,
[46] N-(6-bromobenzo[d]isoxazol-3-yl)-3-fluoro-5-trifluoromethylbenzamide,
[47] N-(6-bromobenzo[d]isoxazol-3-yl)-2,3,4-trifluorobenzamide,
[48] N-(6-bromobenzo[d]isoxazol-3-yl)-4-propylbenzamide,
[49] N-(6-bromobenzo[d]isoxazol-3-yl)-3,4-difluorobenzamide,
[50] furan-2-(5-bromobenzo[d]isoxazol-3-yl)carboxamide,
[51] 7-fluoro-N-(4-fluorobenzo[d]isoxazol-3-yl)-2-trifluoromethylbenzamide,
[52] 2,4-dichloro-5-fluoro-N-(7-fluorobenzo[d]isoxazol-3-yl)benzamide,
[53] 3-fluoro-N-(7-fluorobenzo[d]isoxazol-3-yl)-4-trifluoromethyl benzamide,
[54] 2,3,4-trifluoro-N-(7-fluorobenzo[d]isoxazol-3-yl)benzamide,
[55] N-(7-fluorobenzo[d]isoxazol-3-yl)-4-iodobenzamide,
[56] 2-chloro-N-(7-fluorobenzo[d]isoxazol-3-yl)nicotinamide,
[57] naphthalene-2-(7-fluorobenzo[d]isoxazol-3-yl)carboxamide,
[58] N-(7-fluorobenzo[d]isoxazol-3-yl)-4-propyl benzamide,
[59] 3,4-difluoro-N-(7-fluorobenzo[d]isoxazol-3-yl)benzamide,
[60] 2-fluoro-N-(7-fluorobenzo[d]isoxazol-3-yl)-3-trifluoromethylbenzamide,
[61] N-(7-fluorobenzo[d]isoxazol-3-yl)-3-methoxybenzamide,
[62] N-(7-fluorobenzo[d]isoxazol-3-yl)-2,2-diphenylacetamide,
[63] furan-2-(7-fluorobenzo[d]isoxazol-3-yl)carboxamide,
[64] 2,4-dichloro-N-(4-dimethylaminobenzo[d]isoxazol-3-yl)-5-fluorobenzamide,
[65] N-(4-dimethylaminobenzo[d]isoxazol-3-yl)-2,3,4-trifluorobenzamide,
[66] 2-chloro-N-(4-dimethylaminobenzo[d]isoxazol-3-yl)nicotinamide,
[67] naphthalene-2-(4-dimethylaminobenzo[d]isoxazol-3-yl)carboxamide,
[68] N-(4-dimethylaminobenzo[d]isoxazol-3-yl)-4-propylbenzamide,
[69] N-(4-dimethylaminobenzo[d]isoxazol-3-yl)-3,4-difluorobenzamide,
[70] N-(4-dimethylaminobenzo[d]isoxazol-3-yl)-2-fluoro-3-trifluoromethyl benzamide,
[71] N-(4-dimethylaminobenzo[d]isoxazol-3-yl)-3-methoxybenzamide,
[72] N-(4-dimethylaminobenzo[d]isoxazol-3-yl)-2,2-dimethylpropionamide,
[73] cyclohexane-(4-dimethylaminobenzo[d]isoxazol-3-yl)carboxamide,
[74] N-(4-dimethylaminobenzo[d]isoxazol-3-yl)-2,2-diphenylacetamide,
[75] furan-2-(4-dimethylaminobenzo[d]isoxazol-3-yl)carboxamide,
[76] N-(4-dimethylaminobenzo[d]isoxazol-3-yl)-2,2,3,3,4,4,4-heptafluorobutyramide,
[77] 4-fluoro-N-[4-(2,2,2-trifluoroethoxy)benzo[d]isoxazol-3-yl]-2-trifluoromethylbenzamide,
[78] 2,4-dichloro-5-fluoro-N-[4-(2,2,2-trifluoroethoxy)benzo[d]isoxazol-3-yl]benzamide,
[79] 3-fluoro-N-[4-(2,2,2-trifluoroethoxy)benzo[d]isoxazol-3-yl]-4-trifluoromethyl-benzamide,
[80] 2,3,4-trifluoro-N-[4-(2,2,2-trifluoroethoxy)benzo[d]isoxazol-3-yl]benzamide,
[81] naphthalene-2-[4-(2,2,2-trifluoroethoxy)benzo[d]isoxazol-3-yl]carboxamide,
[82] 3,4-difluoro-N-[4-(2,2,2-trifluoroethoxy)benzo[d]isoxazol-3-yl]benzamide,
[83] 2-fluoro-N-[4-(2,2,2-trifluoroethoxy)benzo[d]isoxazol-3-yl]-3-trifluoromethylbenzamide,
[85] N-benzo[d]isoxazol-3-yl-2-ethylhexanamide,
[86] N-benzo[d]isoxazol-3-yl-2-methylbutyramide,
[87] N-benzo[d]isoxazol-3-yl-2,6-difluorobenzamide,
[88] N-benzo[d]isoxazol-3-yl-3-fluoro-4-trifluoromethyl benzamide,
[89] N-benzo[d]isoxazol-3-yl-2,3,6-trifluorobenzamide,
[90] N-benzo[d]isoxazol-3-yinicotinamide,
[91] 5-methylisoxazole-3-benzo[d]isoxazol-3-ylcarboxamide,
[92] benzo[b]thiophene-3-benzo[d]isoxazol-3-ylcarboxamide,
[93] 2-chloro-N-(7-fluorobenzo[d]isoxazol-3-yl)benzamide,
[94] 4-tert-butyl-N-(7-fluorobenzo[d]isoxazol-3-yl)benzamide,
[95] N-(7-fluorobenzo[d]isoxazol-3-yl)benzamide,
[96] 4-fluoro-N-(7-fluorobenzo[d]isoxazol-3-yl)benzamide,
[97] 2-fluoro-N-(7-fluorobenzo[d]isoxazol-3-yl)benzamide,
[98] N-(7-fluorobenzo[d]isoxazol-3-yl)-2-methoxybenzamide,
[99] N-(7-fluorobenzo[d]isoxazol-3-yl)-2-methylbenzamide,
[100] 3,5-difluoro-N-(6-fluorobenzo[d]isoxazol-3-yl)benzamide,
[101] naphthalene-1-(6-fluorobenzo[d]isoxazol-3-yl)carboxamide,
[102] adamantane-1-(6-fluorobenzo[d]isoxazol-3-yl)carboxamide,
[103] 2-bromo-N-(5-fluorobenzo[d]isoxazol-3-yl)benzamide,
[104] 4-tert-butyl-N-(5-fluorobenzo[d]isoxazol-3-yl)benzamide,
[105] 2,4-difluoro-N-(5-fluorobenzo[d]isoxazol-3-yl)benzamide,
[106] naphthalene-2-(5-fluorobenzo[d]isoxazol-3-yl)carboxamide,
[107] N-(5-fluorobenzo[d]isoxazol-3-yl)-4-propylbenzamide,
[108] 2-chloro-N-(6-chlorobenzo[d]isoxazol-3-yl)benzamide,
[109] 4-tert-butyl-N-(6-chlorobenzo[d]isoxazol-3-yl)benzamide,
[110] N-(6-chlorobenzo[d]isoxazol-3-yl)-4-fluoro-2-trifluoromethylbenzamide,
[111] 2,4-dichloro-N-(6-chlorobenzo[d]isoxazol-3-yl)-5-fluorobenzamide,
[112] N-(6-chlorobenzo[d]isoxazol-3-yl)-2-ethylhexanamide,
[113] N-(6-chlorobenzo[d]isoxazol-3-yl)-2-methylbutyramide,
[114] N-(6-bromobenzo[d]isoxazol-3-yl)-2-chlorobenzamide,
[115] N-(6-bromobenzo[d]isoxazol-3-yl)-3,4-dichlorobenzamide,
[116] thiophene-2-(6-bromobenzo[d]isoxazol-3-yl)carboxamide,
[117] N-(6-bromobenzo[d]isoxazol-3-yl)-3,3-dimethylbutyramide,
[118] N-(5-methylbenzo[d]isoxazol-3-yl)benzamide,
[119] 4-bromo-N-(5-methylbenzo[d]isoxazol-3-yl)benzamide,
[120] 2-chloro-N-(5-methylbenzo[d]isoxazol-3-yl)benzamide,
[121] 4-fluoro-N-(5-methyl benzo[d]isoxazol-3-yl)benzamide,
[122] 2-fluoro-N-(5-methylbenzo[d]isoxazol-3-yl)benzamide,
[123] 3-methyl-N-(5-methylbenzo[d]isoxazol-3-yl)benzamide,
[124] 4-tert-butyl-N-(5-methylbenzo[d]isoxazol-3-yl)benzamide,
[125] 2-methyl-N-(5-methylbenzo[d]isoxazol-3-yl)benzamide,
[126] 2,3-difluoro-N-(5-methylbenzo[d]isoxazol-3-yl)benzamide,
[127] 2,4-difluoro-N-(5-methyl benzo[d]isoxazol-3-yl)benzamide,
[128] 2-chloro-N-(5-methyl benzo[d]isoxazol-3-yl)nicotinamide,
[129] cyclopropane-(5-fluorobenzo[d]isoxazol-3-yl)carboxamide,
[130] N-(5-fluorobenzo[d]isoxazol-3-yl)-3,3-dimethylbutyramide,
[131] 2,4-difluoro-N-(5-fluorobenzo[d]isoxazol-3-yl)benzamide,
[132] 2,5-dimethylfuran-3-(5-fluorobenzo[d]isoxazol-3-yl)carboxamide,
[133] N-(5-bromobenzo[d]isoxazol-3-yl)-2,3-difluorobenzamide,
[134] 2,5-dimethylfuran-3-(5-bromobenzo[d]isoxazol-3-yl)carboxamide,
[135] N-(5-bromobenzo[d]isoxazol-3-yl)-2-methyl butyramide,
[136] N-(5-bromobenzo[d]isoxazol-3-yl)-2,6-difluorobenzamide,
[137] N-(5-bromobenzo[d]isoxazol-3-yl)-3,4-dimethoxybenzamide,
[138] N-(5-bromobenzo[d]isoxazol-3-yl)-4-methylbenzamide,
[139] N-(5-bromobenzo[d]isoxazol-3-yl)-2,6-dimethoxybenzamide,
[140] 2-bromo-N-(5-bromobenzo[d]isoxazol-3-yl)benzamide,
[141] N-(5-bromobenzo[d]isoxazol-3-yl)-5-fluoro-2-trifluoromethylbenzamide,
[142] N-(5-bromobenzo[d]isoxazol-3-yl)-3-methoxybenzamide,
[143] 3-methyl-N-(5-methylbenzo[d]isoxazol-3-yl)benzamide,
[144] 4-cyano-N-(5-methylbenzo[d]isoxazol-3-yl)benzamide,
[145] N-(5-methylbenzo[d]isoxazol-3-yl)-2-phenylbutyramide,
[146] N-(5-methylbenzo[d]isoxazol-3-yl)-2-methylpentamide,
[147] N-(4-fluorobenzo[d]isoxazol-3-yl)benzamide,
[148] 4-chloro-N-(4-fluorobenzo[d]isoxazol-3-yl)benzamide,
[149] 2-fluoro-N-(4-fluorobenzo[d]isoxazol-3-yl)benzamide,
[150] adamantane-1-(4-fluorobenzo[d]isoxazol-3-yl)carboxamide,
[151] adamantane-1-(4-chlorobenzo[d]isoxazol-3-yl)carboxamide,
[152] N-(4-chlorobenzo[d]isoxazol-3-yl)benzamide,
[153] N-(4-chlorobenzo[d]isoxazol-3-yl)-3-methylbenzamide,
[154] N-(4-chlorobenzo[d]isoxazol-3-yl)-2-methyl butyramide,
[155] N-(4-chlorobenzo[d]isoxazol-3-yl)-3,3-dimethyl butyramide,
[156] N-(4-methoxybenzo[d]isoxazol-3-yl)-2-methylbenzamide,
[157] 2-chloro-N-(4-methoxybenzo[d]isoxazol-3-yl)benzamide,
[158] N-(4-methoxybenzo[d]isoxazol-3-yl)-2-trifluoromethylbenzamide,
[159] N-(4-methoxybenzo[d]isoxazol-3-yl)-2-ethylhexanamide,
[160] N-(4-methoxybenzo[d]isoxazol-3-yl)-2-methyl butyramide,
[161] N-(4-methoxybenzo[d]isoxazol-3-yl)-2-methylpentamide,
[162]cyclopropane-[4-(2,2,2-trifluoroethoxy)benzo[d]isoxazol-3-yl]carboxamide,
[163] 2-methyl-N-[4-(2,2,2-trifluoroethoxy)benzo[d]isoxazol-3-yl]butyramide,
[164] 3,3-dimethyl-N-[4-(2,2,2-trifluoroethoxy)benzo[d]isoxazol-3-yl]butyramide,
[165] cyclohexane-[4-(2,2,2-trifluoroethoxy)benzo[d]isoxazol-3-yl]carboxamide,
[166] 2,5-dimethylfuran-3-[4-(2,2,2-trifluoroethoxy)benzo[d]isoxazol-3-yl]carboxamide,
[167] N-[4-(4-methyl benzyloxy)benzo[d]isoxazol-3-yl]-3-nitrobenzamide,
[168] N-[4-(4-methylbenzyloxy)benzo[d]isoxazol-3-yl]-4-propylbenzamide,
[169] N-(4-dimethylaminobenzo[d]isoxazol-3-yl)-3-fluoro-5-trifluoromethylbenzamide,
[170] 3,4-dichloro-N-(4-dimethylaminobenzo[d]isoxazol-3-yl)benzamide,
[171] N-(4-dimethylaminobenzo[d]isoxazol-3-yl)-3-nitrobenzamide,
[172] adamantane-1-(4-dimethylaminobenzo[d]isoxazol-3-yl)carboxamide,
[173] benzo[b]thiophene-2-(4-dimethylaminobenzo[d]isoxazol-3-yl)carboxamide,
[174] N-(4-dimethylaminobenzo[d]isoxazol-3-yl)-3,3-dimethylbutyramide,
[176] N-(6-amino-4,5,7-trifluorobenzo[d]isoxazol-3-yl)-2,6-difluorobenzamide and
[177] N-(6-amino-4,5,7-trifluorobenzo[d]isoxazol-3-yl)-2,4-difluorobenzamide.

The medicaments of the invention are particularly suitable for mGluR5 receptor regulation, preferably for inhibiting the mGluR5 receptor, and/or for noradrenaline receptor regulation, preferably for noradrenaline reuptake inhibition, and/or for serotonin (5-HT) receptor regulation, preferably for serotonin reuptake inhibition, and thus also for the prophylaxis and/or treatment of disorders and diseases which are mediated at least partly by mGluR5 receptors and/or noradrenaline receptors and/or serotonin receptors.

The medicament of the invention is preferably suitable for the treatment and/or prophylaxis of one or more disorders selected from the group consisting of disorders of food intake, preferably selected from the group consisting of bulimia, anorexia, obesity and cachexia;

pain, preferably pain selected from the group consisting of acute pain, chronic pain, visceral pain and neuropathic pain; migraines; chronic paroxysmal hemicrania; depression; urinary incontinence; cough, asthma; glaucoma; tinitus; inflammations; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); epilepsy; narcolepsy; diarrhea; gastritis, gastric ulcer; pruritus; anxiety states; panic attacks; schizophrenia; cerebral ischemia; muscle spasms; cramps; gastroesaphageal reflux syndrome; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency;
for the prophylaxis and/or reduction of a development of tolerance to medicaments and/or drugs, especially medicaments based on opioids; for regulating food intake; for modulating motor activity, for regulating the cardiovascular system; for local anesthesia; for increasing vigilance; for increasing libido; for diuresis and/or for antinatriuresis.

The medicament of the invention is particularly preferably suitable for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraine; depression; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); anxiety states; panic attacks; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency.

The medicament of the invention is very particularly preferably suitable for the treatment and/or prophylaxis of pain, preferably pain selected from the group consisting of acute pain, chronic pain, visceral pain and neuropathic pain.

The present invention further relates to the use of at least one substituted N-benzo[d]isoxazol-3-ylamine derivative of the invention, of the general formula I indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for the manufacture of a medicament for mGluR5 receptor regulation, preferably for inhibiting the mGluR5 receptor, and/or for noradrenaline receptor regulation, preferably for noradrenaline reuptake inhibition, and/or for serotonin (5-HT) receptor regulation, preferably for serotonin reuptake inhibition.

Preference is given to the use of at least one substituted N-benzo[d]isoxazol-3-yl-amine derivative of the invention, of the general formula I indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the prophylaxis and/or treatment of disorders and diseases which are mediated at least partly by mGluR5 receptors and/or noradrenaline receptors and/or serotonin receptors.

Particular preference is given to the use of at least one substituted N-benzo[d]isoxazol-3-ylamine derivative of the invention, of the general formula I indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraines; chronic paroxysmal hemicrania; depression; urinary incontinence; cough, asthma; glaucoma; tinitus; inflammations; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); epilepsy; narcolepsy; diarrhea; gastritis, gastric ulcer; pruritus; anxiety states; panic attacks; schizophrenia; cerebral ischemia; muscle spasms; cramps; gastroesaphageal reflux syndrome; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency; for the prophylaxis and/or reduction of a development of tolerance to medicaments and/or drugs, especially medicaments based on opioids; for regulating food intake; for modulating motor activity, for regulating the cardiovascular system; for local anesthesia; for increasing vigilance; for increasing libido; for diuresis and/or for antinatriuresis.

Very particular preference is given to the use of at least one substituted N-benzo[d]isoxazol-3-ylamine derivative of the invention, of the general formula I indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraine; depression; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); anxiety states; panic attacks; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency.

Yet more preference is given to the use of at least one substituted N-benzo[d]isoxazol-3-ylamine derivative of the invention, of the general formula I indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of pain, preferably of pain selected from the group consisting of acute pain, chronic pain, visceral pain and neuropathic pain.

The medicament of the invention is suitable for administration to adults and children, including infants and babies.

The medicament of the invention can be in the form of a liquid, semisolid or solid pharmaceutical form, for example in the form of solutions for injection, drops, elixirs, syrups, sprays, suspensions, tablets, patches, capsules, plasters, suppositories, ointments, creams, lotions, gels, emulsions, aerosols or in multiparticulate form, for example in the form of pellets or granules, where appropriate compressed to tablets, packed into capsules or suspended in a liquid, and be administered as such. Besides at least one substituted N-benzo[d]isoxazol-3-ylamine derivative of the general formula I indicated above, where appropriate in the form of one of its pure stereoisomers, in particular enantiomers or diastereomers or rotamers, its racemate or in the form of mixtures of stereoisomers, in particular of enantiomers or diastereomers or rotamers, in any mixing ratio, or where appropriate in the form of a corresponding salt or in each case in the form of a corresponding solvate, the medicament of the invention normally comprises further physiologically tolerated pharmaceutical excipients which can preferably be selected from the group consisting of carrier materials, fillers, solvates, diluents, surface-active substances, colorants, preservatives, disintegrants, glidants, lubricants, flavorings and binders.

The selection of the physiologically tolerated excipients and the amounts thereof to be employed depends on whether the medicament is to be administered orally, subcutaneously, parenterally, intravenously, intraperitoneally, intradermally, intramuscularly, intranasally, buccally, rectally or locally, for example on infections of the skin, the mucous membranes and of the eyes. Suitable and preferred for oral administration are preparations in the form of tablets, coated tablets, capsules, granules, pellets, drops, elixiers and syrups, and for parenteral, topical and inhalational administration are solutions, suspensions, easily reconstitutable dry preparations, and sprays.

The substituted N-benzo[d]isoxazol-3-ylamine derivatives of the invention, of the general formula I indicated above, which are employed in the medicament of the invention may be in the form of a depot, in dissolved form or in a plaster, where appropriate with the addition of agents promoting skin penetration, as suitable percutaneous administration preparations.

Formulations which can be used orally or percutaneously may also release the particular substituted N-benzo[d]isoxazol-3-ylamine derivatives of the invention, of the general formula I indicated above, in a delayed manner. It is possible in principle to add to the medicament of the invention other further active ingredients known to the skilled worker.

The medicaments of the invention are manufactured with the aid of conventional means, apparatuses, methods and processes known from the prior art, as described for example in “Remington's Pharmaceutical Sciences”, edited by A. R. Gennaro, 17^{th} edition, Mack Publishing Company, Easton, Pa., 1985, especially in part 8, chapter 76 to 93. The corresponding description is introduced hereby as reference and forms part of the disclosure.

The amount of the particular substituted N-benzo[d]isoxazol-3-ylamine derivatives of the invention, of the general formula I indicated above, to be administered to the patient may vary and depends for example on the weight or age of the patient and on the mode of administration, the indication and the severity of the disorder. Normally, 0.005 to 100 mg/kg, preferably 0.05 to 75 mg/kg, of the patient's body weight of at least one such compound of the invention are administered.

The present application further relates to substituted N-benzo[d]isoxazol-3-ylamine derivatives of the general formula Ia,

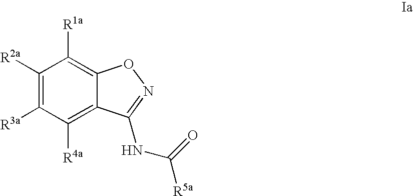

in which
R^{1a} is H, F, Cl, Br, I, —CN, —NC, —SO₃H, —S(═O₂)NH₂, —OH, —SH, is a linear or branched C₁₋₁₀ alkyl radical, —OR⁶, —O—(CH₂)—R^{7a}, —SR^{8a}, —S—(CH₂)—R^{9a}, NR^{10a}R^{11a}, —NH—C(═O)—R^{12a} or —NR^{13a}—C(═O)—R^{12a};
R^{2a} is H, F, Cl, Br, I, —CN, —NC, —SO₃H, —S(═O₂)NH₂, —NH₂, —OH, —SH, is a linear or branched C₁₋₁₀ alkyl radical, —OR^{6a}, —O—(CH₂)—R^{7a}, —SR^{8a}, —S—(CH₂)—R^{9a} or —NR^{10a}R^{11a};
R^{3a} is H, F, Cl, Br, I, —CN, —NC, —SO₃H, —S(═O₂)NH₂, —OH, —SH, is a linear or branched C₁₋₁₀ alkyl radical, —OR^{6a}, —O—(CH₂)—R^{7a}, —SR^{8a}, —S—(CH₂)—R^{9a} or NR^{10a}R^{11a};
R^{4a} is H, F, Cl, Br, I, —CN, —NC, —NO₂, —SO₃H, —S(═O₂)NH₂, —NH₂, —SH, is a linear or branched C₁₋₁₀alkyl radical, —OR⁶, —O—(CH₂)—R^{7a}, —SR^{8a}, —S—(CH₂)—R^{9a}, NR^{10a}R^{11a}, —NH—C(═O)—R^{12a} or —NR^{13a}—C(═O)—R^{12a};
R^{5a} is a linear or branched C₂₋₁₀ alkyl radical;
   is CₘF₂ₘ₊₁ with m=1, 2, 3, 4 or 5;
   is a linear or branched C₂₋₁₀ alkenyl radical;
   is an unsaturated or saturated, optionally substituted 3-, 4-, 5-, 6-, 7-, 8- or 9-membered cycloaliphatic radical which may be bridged by 1 or 2 linear or branched C₁₋₅-alkylene groups;
   is an imidazolidinonyl radical which may be substituted by 1 or 2 substituents independently of one another selected from the group consisting of methyl, ethyl, n-propyl, isopropyl, n-butyl and tert-butyl;
   is a phenyl radical which may be substituted in each case by 1, 2, 3, 4 or 5 substituents independently of one another selected from the group consisting of F, Cl, Br, I, —CN, —CF₃, —SF₅, —OH, —O—C₁₋₅-alkyl, —O—C₂₋₅-alkenyl, —NH₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl and benzyl,
   where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl and benzyl may be substituted by 1, 2, 3, 4 or 5 substituents independently of one another selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl,
   with the proviso that at least one of the meta positions and/or the para position of the phenyl radical is substituted by one of the aforementioned substituents, where the ortho positions of the phenyl radical are unsubstituted or substituted by at least one substituent selected from the group consisting of F, Cl, Br, methyl and —O—CH₃,
   and the radicals R¹, R², R³ and R⁴ are each H, or one of the radicals R¹, R², R³ and R⁴ is F, Cl, Br, I, —C₁₋₃-alkyl, —CF₃, —C₂F₅, —OCF₃, —OC₂F₅, —CF₂H, —C₂F₄H, —OCF₂H or —OC₂F₄H, and the remaining ones of these radicals in each case are H;
   is a radical selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl and 4-methyl-3-nitrophenyl;
   is an optionally substituted naphthyl radical;
   is an optionally substituted 6- to 10-membered aryl radical which is fused to a saturated or unsaturated, optionally substituted mono- or polycyclic ring system;
   is an optionally substituted 5- to 14-membered heteroaryl radical;
   is —(CH₂)ₙ—C(═O)—OR^{14a} with n=0, 1, 2, 3, 4 or 5;
   is —CR^{15a}R^{16a}—O—C(═O)—R^{17a};
   is —(CHR^{18a})—(CH₂)ₚₐ—R^{19a} with pa=0 or 1;
   or
   is —(CH═CH)—R^{20a};
R^{6a} and R^{8a} independently of one another each
   are a linear or branched, optionally substituted C₁₋₁₀ alkyl radical
   or are an optionally substituted 5- to 14-membered aryl or heteroaryl radical;
R^{7a} and R^{9a} independently of one another each
   are an optionally substituted 5- to 14-membered aryl or heteroaryl radical;
R^{10a} and R^{11a} independently of one another each
   are a hydrogen radical or
   are a linear or branched C₁₋₁₀ alkyl radical;
R^{12a}, R^{13a}, R^{14a} and R^{17a} independently of one another each
   are a linear or branched C₁₋₁₀ alkyl radical;
R^{15a} is a linear or branched C₁₋₁₀ alkyl radical
   or an optionally substituted 6- to 10-membered aryl radical;
R^{16a} and R^{18a} independently of one another each
   are a hydrogen radical;
   are a linear or branched C₁₋₁₀ alkyl radical
   or
   are an optionally substituted 6- to 10-membered aryl radical;
R^{19a} is an unsaturated or saturated, optionally substituted 3-, 4-, 5-, 6-, 7-, 8- or 9-membered cycloaliphatic radical,
   is an optionally substituted 6- to 10-membered aryl radical,
   or
   is an optionally substituted thiophenyl or furanyl radical;
      and
R^{20a} is an optionally substituted 6- to 10-membered aryl radical;
   in each case where appropriate in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

The aforementioned optionally substituted cycloaliphatic radicals may preferably each be unsubstituted or optionally substituted in each case by 1, 2, 3, 4 or 5 substituents independently of one another selected from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—H, —C(═O)—C₁₋₅-alkyl, —C(═O)—O—C₁₋₅-alkyl, —(CH₂)—C(═O)—O—C₁₋₅-alkyl, —NH—C₁₋₅-alkyl, —N(C₁₋₅-alkyl)₂, —(CH₂)-benzo[b]furanyl, —O-phenyl, —O-benzyl, phenyl, benzyl, naphthyl and —(CH₂)-naphthyl, where in each case the cyclic part of the radicals —O-phenyl, —O-benzyl, phenyl, —(CH₂)-benzo[b]furanyl, benzyl, naphthyl and —(CH₂)-naphthyl may be substituted by 1, 2, 3, 4 or 5 substituents independently of one another selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl.

Where the substituent R^{5a} and/or R^{19a} is a cycloaliphatic radical which may optionally be bridged by 1 or 2 linear or branched C₁₋₅-alkylene groups, this can preferably be selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantyl.

The cycloaliphatic radicals may particularly preferably each optionally be substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂, —N(CH(CH₃)₂)₂, —(CH₂)-benzo[b]furanyl, —O-phenyl, —O-benzyl, phenyl, benzyl, naphthyl and —(CH₂)-naphthyl, where in each case the cyclic part of the radicals —O-phenyl, —O-benzyl, phenyl, —(CH₂)-benzo[b]furanyl, benzyl, naphthyl and —(CH₂)-naphthyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇, methyl, ethyl, n-propyl, isopropyl, n-butyl, sec-butyl, isobutyl, tert-butyl, n-pentyl, 2-pentyl, 3-pentyl, neopentyl, —O—CF₃, —S—CF₃, phenyl and —O-benzyl.

Unless indicated otherwise, the aforementioned optionally substituted aryl, heteroaryl, naphthyl, furanyl or thiophenyl radicals can likewise preferably each be unsubstituted or optionally each substituted by 1, 2, 3, 4 or 5 substituents independently of one another selected from the group consisting of F, Cl, Br, I, —CN, —CF₃, —SF₅, —OH, —O—C₁₋₅-alkyl, —O—C₂₋₅-alkenyl, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl may be substituted by 1, 2, 3, 4 or 5 substituents independently of one another selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl.

The aforementioned heteroaryl radicals may preferably optionally each have 1, 2, 3, 4 or 5 heteroatom(s) independently of one another selected from the group consisting of oxygen, nitrogen and sulfur as ring member(s).

Where one or more of the substituents R^{5a} to R^{9a}, R^{15a}, R^{16a} and R^{18a} to R^{20a} are an aryl radical, this can preferably be selected from the group consisting of phenyl and naphthyl (1-naphthyl and 2-naphthyl).

The aryl radicals can particularly preferably optionally each be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CH₂—CH═CH₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —C(═O)—H, —C(═O)—CH₃, —C(═O)—C₂H₅, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —(CH₂)-benzo[b]furanyl, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇, methyl, ethyl, n-propyl, isopropyl, n-butyl, sec-butyl, isobutyl, tert-butyl, n-pentyl, 2-pentyl, 3-pentyl, neopentyl, —O—CF₃, —S—CF₃, phenyl and —O-benzyl.

Where one or more of the substituents R^{5a} to R^{9a} and R^{19a} are a heteroaryl radical or include one such, this can preferably be selected from the group consisting of thiophenyl, furanyl, pyrrolyl, pyrazolyl, pyranyl, triazolyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl.

The heteroaryl radicals may particularly preferably optionally each be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CH₂—CH═CH₂, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —C(═O)—H, —C(═O)—CH₃, —C(═O)—C₂H₅, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, —(CH₂)-benzo[b]furanyl, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇, methyl, ethyl, n-propyl, isopropyl, n-butyl, sec-butyl, isobutyl, tert-butyl, n-pentyl, 2-pentyl, 3-pentyl, neopentyl, —O—CF₃, —S—CF₃, phenyl and —O-benzyl.

The aforementioned optionally substituted C₁₋₁₀ alkyl radicals may likewise preferably each be unsubstituted or optionally each substituted by 1, 2, 3, 4 or 5 substituents independently of one another selected from the group consisting of F, Cl, Br, I, —OH, —SH, —CN and —NO₂.

Where one or more of the substituents R^{1a} to R^{4a}, R^{10a} to R^{18a} are a linear or branched C₁₋₁₀-alkyl radical, this can preferably be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

If R^{5a} is a linear or branched C₂₋₁₀-alkyl radical, this can preferably be selected from the group consisting of ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

If the substituent R^{6a} and/or R^{8a} is a linear or branched, optionally substituted C₁₋₁₀-alkyl radical, this can preferably be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl and optionally each be substituted by 1, 2, 3, 4 or 5 substituents independently of one another selected from the group consisting of F, Cl, Br, I, —CN and —NO₂.

Particularly preferred optionally substituted C₁₋₁₀-alkyl radicals can be selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃ and —CH₂—CH₂—CH₂—CN.

If R^{5a} is a C₂₋₁₀-alkenyl radical, this radical can preferably be selected from the group consisting of vinyl, 1-propenyl, 2-propenyl, 1-butenyl, 2-butenyl, 3-butenyl, 2-methyl-1-propenyl, 1-pentenyl, 2-pentenyl and 3-pentenyl.

A mono- or polycyclic ring system means in the context of the present invention mono- or polycyclic hydrocarbon radicals which may be saturated or unsaturated and optionally have 1, 2, 3, 4 or 5 heteroatom(s) as ring member(s) which are selected independently of one another from the group consisting of oxygen, nitrogen and sulfur.

Such a mono- or polycyclic ring system can for example be fused to an aryl radical or a heteroaryl radical.

Where a polycyclic ring system such as, for example, a bicyclic ring system is present, the various rings may each independently of one another have a different degree of saturation, i.e. be saturated or unsaturated. A polycyclic ring system is preferably a bicyclic ring system.

The rings of the aforementioned mono- or polycyclic ring systems preferably each have 5, 6 or 7 members and may each have 1, 2 or 3 heteroatom(s) as ring member(s) which are selected independently of one another from the group consisting of oxygen, nitrogen and sulfur.

The rings of the aforementioned optionally substituted mono- or polycyclic ring systems may preferably be unsubstituted or optionally substituted by 1, 2, 3, 4 or 5 substituents independently of one another selected from the group consisting of oxo (═O), thioxo (═S), F, Cl, Br, I, —CN, —CF₃, —SF₅, —OH, —O—C₁₋₅-alkyl, —O—C₂₋₅-alkenyl, —NH₂, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl and —S(═O)₂—C₁₋₅-alkyl.

The rings of the aforementioned optionally substituted mono- or polycyclic ring systems may particularly preferably be unsubstituted or optionally each substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —C(═O)—H, —C(═O)—CH₃, —C(═O)—C₂H₅, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅ and —S(═O)₂—CH(CH₃)₂.

Examples which may be mentioned of aryl radicals which are fused to a mono- or polycyclic ring system are [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl.

Where the radical R^{5a} includes a linear or branched C₁₋₅-alkylene group, this can preferably be selected from the group consisting of —(CH₂)—, —(CH₂)₂—, —C(H)(CH₃)—, —C(CH₃)₂—, —(CH₂)₃—, —(CH₂)₄—, —(CH₂)₅—, —C(H)(C(H)(CH₃)₂)— and —C(C₂H₅)(H)—.

Preferred substituted N-benzo[d]isoxazol-3-ylamine derivatives of the general formula Ia indicated above are those in which
R^{6a} and R^{8a} independently of one another each
   are a radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃ and —CH₂—CH₂—CH₂—CN;
   or are a radical selected from the group consisting of phenyl, naphthyl, thiophenyl, furanyl and pyridinyl, where the radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and R^{1a} to R^{5a}, R^{7a} and R^{9a} to R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred substituted N-benzo[d]isoxazol-3-ylamine derivatives of the general formula Ia indicated above are those in which
R^{7a} and R^{9a} independently of one another each
   are a radical selected from the group consisting of phenyl, naphthyl, thiophenyl, furanyl and pyridinyl, where the radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and R^{1a} to R^{6a}, R^{8a} and R^{10a} to R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred substituted N-benzo[d]isoxazol-3-ylamine derivatives of the general formula Ia indicated above are those in which
R^{10a} and R^{11a} independently of one another each are a hydrogen radical
   or are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl;
      and R^{1a} to R^{9a} and R^{12a} to R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred substituted N-benzo[d]isoxazol-3-ylamine derivatives of the general formula Ia indicated above are those in which
R^{12a}, R^{13a}, R^{14a} and R¹⁷, independently of one another each are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl;
   and R^{1a} to R^{11a}, R^{15a}, R^{16a} and R^{18a} to R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred substituted N-benzo[d]isoxazol-3-ylamine derivatives of the general formula Ia indicated above are those in which
R^{15a} is an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl
   or is a phenyl or naphthyl radical, where the radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and R^{1a} to R^{14a} and R^{16a} to R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred substituted N-benzo[d]isoxazol-3-ylamine derivatives of the general formula Ia indicated above are those in which
R^{16a} and R^{18a} independently of one another each
   are a hydrogen radical;
   are an alkyl radical selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl
   or are a phenyl or naphthyl radical, where the radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and R^{1a} to R^{15a}, R^{17a}, R^{19a} and R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Further preferred substituted N-benzo[d]isoxazol-3-ylamine derivatives of the general formula Ia indicated above are those in which
R^{19a} is a cycloaliphatic radical selected from the group consisting of cyclopropyl, cyclobutyl, cyclopentyl, cyclohexyl, cycloheptyl, cyclopentenyl, cyclohexenyl and cycloheptenyl, where the cycloaliphatic radical may in each case optionally be substituted by 1, 2, 3, 4 or 5 substituents independently of one another selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂ and —N(CH(CH₃)₂)₂;
   or is a radical selected from the group consisting of phenyl, naphthyl, furanyl and thiophenyl, where the radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
      and R^{1a} to R^{18a} and R^{20a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Likewise preferred substituted N-benzo[d]isoxazol-3-ylamine derivatives of the general formula Ia indicated above are those in which
R^{20a} is a phenyl or naphthyl radical, where the radical may in each case be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl;
   and R^{1a} to R^{19a} have the aforementioned meaning, in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Particularly preferred substituted N-benzo[d]isoxazol-3-ylamine derivatives of the general formula Ia indicated above are those in which
R^{1a} is H, F, Cl, Br, I, —CN, —NC, —SO₃H, —S(═O₂)NH₂, —OH, —SH, methyl, ethyl, n-propyl, isopropyl, n-butyl, sec-butyl, isobutyl, tert-butyl, n-pentyl, sec-pentyl, n-hexyl, —OR⁶, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{2a} is H, F, Cl, Br, I, —CN, —NC, —SO₃H, —S(═O₂)NH₂, —NH₂, —OH, —SH, methyl, ethyl, n-propyl, isopropyl, n-butyl, sec-butyl, isobutyl, tert-butyl, n-pentyl, sec-pentyl, n-hexyl, —OR⁶, —O—(CH₂)—R^{7a}, —SR^{8a}, S—(CH₂)—R^{9a} or NR^{10a}R^{11a};
R^{3a} is H, F, Cl, Br, I, —CN, —NC, —SO₃H, —S(═O₂)NH₂, —OH, —SH, methyl, ethyl, n-propyl, isopropyl, n-butyl, sec-butyl, isobutyl, tert-butyl, n-pentyl, sec-pentyl, n-hexyl —OR^{6a}—O—(CH₂)—R^{7a}, SR^{8a}, —S—(CH₂)—R^{9a} or —NR^{10a}R^{11a};
R^{4a} is H, F, Cl, Br, I, —CN, —NC, —NO₂, —SO₃H, —S(═O₂)NH₂, —NH₂, —SH, methyl, ethyl, n-propyl, isopropyl, n-butyl, sec-butyl, isobutyl, tert-butyl, n-pentyl, sec-pentyl, n-hexyl, —OR^{6a}, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{5a} is a radical selected from the group consisting of ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CF₂—CF₃, —CF₂—CF₂—CF₃, —CF₂—CF₂—CF₂—CF₃, 1-propenyl, 2-propenyl, 1-butenyl, 2-butenyl, 3-butenyl and 2-methyl-1-propenyl;
   is a cycloaliphatic radical selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantyl, where the cycloaliphatic radical may in each case optionally be substituted by 1, 2, 3, 4 or 5 substituents independently of one another selected from the group consisting of oxo (═O), thioxo (═S), F, Cl, Br, I, —CN, —CF₃, —SF₅, —O—CH₃, —O—C₂H₅, —O—C₃H₇, methyl, ethyl, n-propyl, isopropyl, n-butyl, sec-butyl, isobutyl, tert-butyl, phenyl and benzyl, where in each case the cyclic part of the radicals phenyl and benzyl may be substituted by 1, 2, 3, 4 or 5 substituents independently of one another selected from the group consisting of F, Cl, Br, methyl, ethyl and n-propyl;
   is an imidazolidinonyl radical;
   is a phenyl radical, where the radical may in each case be substituted by 1, 2, 3, 4 or 5 substituents independently of one another selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, phenyl and benzyl, where in each case the cyclic part of the radicals phenyl and benzyl may be substituted by 1, 2, 3, 4 or 5 substituents independently of one another selected from the group consisting of F, Cl, Br, —OH, —CF₃, —SF₅, —CN, —NO₂, —O—CH₃, —O—C₂H₅, —O—C₃H₇ methyl, ethyl, —O—CF₃ and —S—CF₃;
   is a radical selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl and 4-methyl-3-nitrophenyl;
   is a radical selected from the group consisting of naphthyl, [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl, where the radical may in each case be substituted by 1, 2, 3, 4 or 5 substituents independently of one another selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl and tert-butyl;
   is a heteroaryl radical selected from the group consisting of thiophenyl, furanyl, pyrazolyl, pyrazinyl, pyranyl, triazolyl, pyridinyl, indolyl, benzo[b]furanyl, benzo[b]thiophenyl, thiazolyl, oxazolyl, isoxazolyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl, where the heteroaryl radical may in each case optionally be substituted by 1, 2, 3, 4 or 5 substituents independently of one another selected from the group consisting of F, Cl, Br, I, —CN, —CF₃, —SF₅, —OH, —O—CH₃, —O—C₂H₅, —O—C₃H₇, —O—CH₂—CH═CH₂, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, dihydrobenzo[b]furanyl, —O-phenyl, —O-benzyl, —S-phenyl, —S-benzyl, phenyl, pyridinyl and benzyl, where in each case the cyclic part of the radicals —S(═O)₂-phenyl, —O-phenyl, —O-benzyl, —S-phenyl, —S-benzyl, phenyl, dihydro[b]benzofuranyl, pyridinyl and benzyl may be substituted by 1, 2, 3, 4 or 5 substituents independently of one another selected from the group consisting of F, Cl, Br, —CF₃, —CN, —NO₂, —O—CH₃, —O—C₂H₅, —O—C₃H₇, methyl, ethyl, n-propyl, isopropyl, n-butyl, sec-butyl, isobutyl, tert-butyl, —O—CF₃ and —S—CF₃;
   is —(CH₂)ₙ—C(═O)—OR^{14a} with n=0, 1, 2, 3 or 4;
   is —CR^{15a}R^{16a}—O—C(═O)—R^{17a};
   is —(CHR^{18a})—(CH₂)ₚₐ—R^{19a} with pa=0 or 1;
   or
   is —(CH═CH)—R^{20a};
R^{6a} and R^{8a} independently of one another each
   are a radical selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R^{7a} and R^{9a} independently of one another each
   are a phenyl radical which may be substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
R^{10a} and R^{11a} independently of one another each
   are an alkyl radical selected from the group consisting of methyl, ethyl, n-propyl, isopropyl, n-butyl, isobutyl and tert-butyl;
R^{12a}, R^{13a}, R^{14a} and R^{17a} independently of one another each
   are an alkyl radical selected from the group consisting of methyl, ethyl, n-propyl, isopropyl and tert-butyl;
R^{15a} is an alkyl radical selected from the group consisting of methyl, ethyl, n-propyl, isopropyl and n-butyl,
   or is a phenyl radical;
R^{16a} is a hydrogen radical,
   or is an alkyl radical selected from the group consisting of methyl, ethyl, n-propyl, isopropyl, n-butyl, isobutyl and tert-butyl;
R^{18a} is a hydrogen radical;
   is an alkyl radical selected from the group consisting of methyl, ethyl, n-propyl, isopropyl, n-butyl, isobutyl and tert-butyl,
   or is a phenyl radical;
R^{19a} is a cycloaliphatic radical selected from the group consisting of cyclopropyl, cyclobutyl, cyclopentyl, cyclohexyl, cycloheptyl, cyclopentenyl, cyclohexenyl and cycloheptenyl;
   is a phenyl radical which may be substituted by 1, 2, 3, 4 or 5 substituents independently of one another selected from the group consisting of F, Cl, Br, I, —CN, —CF₃, —SF₅, —O—CH₃, —O—C₂H₅, —O—C₃H₇, —NO₂, —O—CF₃, —O—CHF₂, —O—CH₂F, methyl, ethyl, n-propyl, isopropyl, n-butyl, sec-butyl, isobutyl and tert-butyl;
   or is a thiophenyl radical;
      and
R^{20a} is a phenyl radical which may be substituted by 1, 2, 3, 4 or 5 substituents independently of one another selected from the group consisting of F, Cl, Br, —CF₃, methyl, ethyl, n-propyl, isopropyl, n-butyl, sec-butyl, isobutyl and tert-butyl.

Very particularly preferred substituted N-benzo[d]isoxazol-3-ylamine derivatives of the general formula Ia indicated above are those in which
R^{1a} is H, F, Cl or Br;
R^{2a} is H, F, Cl, Br or —NH₂;
R^{3a} is H, F, Cl, Br, methyl, ethyl or n-propyl;
R^{4a} is H, F, Cl, Br, —NH₂, methyl, ethyl, n-propyl, isopropyl, n-butyl, isobutyl, tert-butyl, —OR^{6a}, —O—(CH₂)—R^{7a} or —NR^{10a}R^{11a};
R^{5a} is a radical selected from the group consisting of ethyl, n-propyl, isopropyl, n-butyl, sec-butyl, isobutyl, tert-butyl, n-pentyl, 2-pentyl, 3-pentyl, neopentyl, n-hexyl, 3-heptyl, —CF₃, —CF₂—CF₃, —CF₂—CF₂—CF₃ and —CF₂—CF₂—CF₂—CF₃;
   is a cycloaliphatic radical selected from the group consisting of cyclopropyl, cyclobutyl, cyclopentyl, cyclohexyl, cycloheptyl, cyclopentenyl, cyclohexenyl, cycloheptenyl, (6,6)-dimethyl-[3.1.1]-bicycloheptyl and adamantyl;
   is a radical selected from the group consisting of 2-trifluoromethylphenyl, 2-nitrophenyl, 2-dimethylaminophenyl, 2-diethylaminophenyl, 2-aminophenyl, 2-ethylphenyl, 2-methylbenzoate, 2-bromophenyl, 2-chlorophenyl, 2-fluorophenyl, 2-methylphenyl, 2-methoxyphenyl, 2-ethoxyphenyl, 2-cyanophenyl, 2-acetylphenyl, 2-dimethylaminosulfonylphenyl, 3-chlorophenyl, 3-methylphenyl, 3-nitrophenyl, 3-trifluoromethylphenyl, 3-fluorophenyl, 3-bromophenyl, 3-dimethylaminophenyl, 3-diethylaminophenyl, 3-aminophenyl, 3-methoxyphenyl, 3-ethylphenyl, 3-ethoxyphenyl, 3-cyanophenyl, 3-acetylphenyl, 3-phenylphenyl, 3-dimethylaminosulfonylphenyl, 4-bromophenyl, 4-methoxyphenyl, 4-chlorophenyl, 4-fluorophenyl, 4-tert-butylphenyl, 4-cyanophenyl, 4-nitrophenyl, 4-methylphenyl, 4-phenylphenyl, 4-trifluoromethylphenyl, 4-dimethylaminophenyl, 4-diethylaminophenyl, 4-aminophenyl, 4-iodophenyl, 4-n-propylphenyl, 4-di-n-propylaminosulfonylphenyl, 4-diethylaminosulfonylphenyl, 4-dimethylaminosulfonylphenyl, 4-ethylphenyl, 4-ethoxyphenyl, 4-methylbenzoate, 4-acetylphenyl, 2-fluoro-3-trifluoromethylphenyl, (2,3)-difluorophenyl, (2,3)-dimethylphenyl, (2,3)-dichlorophenyl, 3-fluoro-2-trifluoromethylphenyl, (2,4)-dichlorophenyl, (2,4)-difluorophenyl, 4-fluoro-2-trifluoromethylphenyl, (2,4)-dimethoxyphenyl, 2-chloro-4-fluorophenyl, (2,4)-dibromophenyl, 2-fluoro-4-trifluoromethylphenyl, (2,5)-difluorophenyl, 2-fluoro-5-trifluoromethylphenyl, 5-fluoro-2-trifluoromethylphenyl, 5-chloro-2-trifluoromethylphenyl, 5-bromo-2-trifluoromethylphenyl, (2,5)-dimethoxyphenyl, (2,5)-bis-trifluoromethylphenyl, (2,5)-dichlorophenyl, (2,5)-dibromophenyl, 2-fluoro-6-trifluoromethylphenyl, (2,6)-dimethoxyphenyl, (2,6)-dimethylphenyl, 2-chloro-6-fluorophenyl, 2-bromo-6-chlorophenyl, 2-bromo-6-fluorophenyl, (2,6)-difluorophenyl, (2,6)-dibromophenyl, (2,6)-dichlorophenyl, (3,4)-dichlorophenyl, 4-chloro-3-nitrophenyl, (3,4)-dimethoxyphenyl, 4-fluoro-3-trifluoromethylphenyl, 3-fluoro-4-trifluoromethylphenyl, (3,4)-difluorophenyl, 4-bromo-3-methylphenyl, 4-bromo-5-methylphenyl, 3-chloro-4-fluorophenyl, 4-fluoro-3-nitrophenyl, 4-bromo-3-nitrophenyl, (3,4)-dibromophenyl, 4-chloro-3-methylphenyl, 4-fluoro-3-methylphenyl, 4-methyl-3-nitrophenyl, (3,5)-dimethoxyphenyl, (3,5)-bis-trifluoromethylphenyl, (3,5)-difluorophenyl, (3,5)-dinitrophenyl, (3,5)-dichlorophenyl, 3-fluoro-5-trifluoromethylphenyl, 5-fluoro-3-trifluoromethylphenyl, (3,5)-dibromophenyl, 5-chloro-4-fluorophenyl, 5-chloro-4-fluorophenyl, 5-bromo-4-methylphenyl, (2,3,4)-trifluorophenyl, (2,3,4)-trichlorophenyl, (2,3,6)-trifluorophenyl, (2,4,6)-trichlorophenyl, (2,4,5)-trifluorophenyl, (2,4,5)-trichlorophenyl, (2,4)-dichloro-5-fluorophenyl, (2,4,6)-trichlorophenyl, (2,4,6)-trimethylphenyl, (2,4,6)-trifluorophenyl, (2,4,6)-trimethoxyphenyl, (3,4,5)-trimethoxyphenyl, (2,3,4,5)-tetrafluorophenyl and (2,3,4,5,6)-pentafluorophenyl;
   is a naphthyl radical;
   is a heteroaryl radical selected from the group consisting of thiophenyl, furanyl, pyridinyl, benzo[b]furanyl, benzo[b]thiophenyl, oxazolyl and isoxazolyl, where the heteroaryl radical may in each case be optionally substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
   is —(CH₂)ₙ—C(═O)—OR^{14a} with n=1 or 2
   or
   is —(CHR^{18a})—(CH₂)ₚₐ—R^{19a} with p=0 or 1;
R^{6a} is a radical selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R^{7a} is a phenyl radical which may be substituted by 1, 2, 3, 4 or 5 substituents independently of one another selected from the group consisting of F, Cl, Br, methyl, ethyl, n-propyl, isopropyl, n-butyl, sec-butyl, isobutyl and tert-butyl;
R^{10a} and R^{11a} independently of one another each are an alkyl radical selected from the group consisting of methyl, ethyl, n-propyl, isopropyl, n-butyl, isobutyl and tert-butyl;
R^{14a} is an alkyl radical selected from the group consisting of methyl, ethyl, n-propyl, isopropyl, n-butyl, isobutyl and tert-butyl;
R^{18a} is a hydrogen radical;
   is an alkyl radical selected from the group consisting of methyl, ethyl, n-propyl, isopropyl, n-butyl, isobutyl and tert-butyl
   or is a phenyl radical,
   and
R^{19a} is a cycloaliphatic radical selected from the group consisting of cyclopropyl, cyclobutyl, cyclopentyl, cyclohexyl, cycloheptyl, cyclopentenyl, cyclohexenyl and cycloheptenyl;
   is a phenyl radical which may be substituted by 1, 2, 3, 4 or 5 substituents independently of one another selected from the group consisting of F, Cl, Br, I, —CN, —CF₃, —SF₅, —O—CH₃, —O—C₂H₅, —O—C₃H₇, —NO₂, —O—CF₃, —O—CHF₂, —O—CH₂F, methyl, ethyl, n-propyl, isopropyl, n-butyl, sec-butyl, isobutyl and tert-butyl;
   or is a thiophenyl radical;
   in each case optionally in the form of one of their pure stereoisomers, in particular enantiomers or diastereomers or rotamers, their racemates or in the form of a mixture of stereoisomers, in particular of enantiomers and/or diastereomers and/or rotamers, in any mixing ratio, or in each case in the form of corresponding salts, or in each case in the form of corresponding solvates.

Even more preferred substituted N-benzo[d]isoxazol-3-ylamine derivatives of the general formula Ia indicated above are selected from the group consisting of
[1] thiophene-2-benzo[d]isoxazol-3-ylcarboxamide,
[2] N-benzo[d]isoxazol-3-ylsuccinic acid methyl ester
[3] naphthalene-2-benzo[d]isoxazol-3-ylcarboxamide,
[4] adamantane-2-benzo[d]isoxazol-3-ylcarboxamide,
[5] cyclohexane-benzo[d]isoxazol-3-ylcarboxamide,
[6] N-benzo[d]isoxazol-3-yl-2,2-dimethylpropionamide,
[7] N-(4-methoxybenzo[d]isoxazol-3-yl)-2,2-diphenylacetamide,
[8] cyclopropane(5-bromobenzo[d]isoxazol-3-yl)carboxamide,
[9] 3,5-dichloro-N-(6-fluorobenzo[d]isoxazol-3-yl)benzamide,
[10] N-(4-aminobenzo[d]isoxazol-3-yl)-4-nitrobenzamide,
[11] naphthalene-1-(4-aminobenzo[d]isoxazol-3-yl)carboxamide,
[12] benzo[b]thiophene-2-(4-fluorobenzo[d]isoxazol-3-yl)carboxamide,
[13] N-benzo[d]isoxazol-3-yl-2-trifluoromethylbenzamide,
[14] [14] N-benzo[d]isoxazol-3-yl-3,4-dichlorobenzamide
[15] N-benzo[d]isoxazol-3-yl-2,3-difluorobenzamide,
[16] N-(4-dimethylaminobenzo[d]isoxazol-3-yl)-3-fluorobenzamide,
[17] thiophene-2-(6-fluorobenzo[d]isoxazol-3-yl)carboxamide,
[18] [18] N-(6-fluorobenzo[d]isoxazol-3-yl)-2-methylbutyramide,
[19] N-(6-chlorobenzo[d]isoxazol-3-yl)-2-fluoro-3-trifluoromethylbenzamide,
[20] N-(6-chlorobenzo[d]isoxazol-3-yl)-3-methoxybenzamide,
[21] N-(6-bromobenzo[d]isoxazol-3-yl)-4-tert-butylbenzamide,
[23] N-(6-bromobenzo[d]isoxazol-3-yl)-2-trifluoromethylbenzamide,
[24] adamantane-2-(6-bromobenzo[d]isoxazol-3-yl)carboxamide,
[25] N-(6-bromobenzo[d]isoxazol-3-yl)-2-methylbutyramide,
[26] N-(5-fluorobenzo[d]isoxazol-3-yl)-2-trifluoromethyl benzamide,
[27] 3,4-dichloro-N-(5-fluorobenzo[d]isoxazol-3-yl)benzamide,
[29] N-(5-bromobenzo[d]isoxazol-3-yl)-2-fluoro-5-trifluoromethylbenzamide,
[30] N-(5-bromobenzo[d]isoxazol-3-yl)-2,4-difluorobenzamide,
[31] N-(5-methylbenzo[d]isoxazol-3-yl)-2-trifluoromethyl benzamide,
[32] 3 -fluoro-N-(5-methylbenzo[d]isoxazol-3-yl)benzamide,
[33] N-(4-methoxybenzo[d]isoxazol-3-yl)-3,5-bis-trifluoromethylbenzamide,
[34] 3,5-difluoro-N-(4-methoxybenzo[d]isoxazol-3-yl)benzamide,
[35] N-(4-dimethylaminobenzo[d]isoxazol-3-yl)-3,5-bis-trifluoromethylbenzamide,
[36] 2-bromo-N-[4-(2,2,2-trifluoroethoxy)benzo[d]isoxazol-3-yl]benzamide,
[37] 3-chloro-N-[4-(2,2,2-trifluoroethoxy)benzo[d]isoxazol-3-yl] benzamide,
[38] 4-tert-butyl-N-[4-(2,2,2-trifluoroethoxy)benzo[d]isoxazol-3-yl]benzamide,
[39] 2-methoxy-N-[4-(2,2,2-trifluoroethoxy)benzo[d]isoxazol-3-yl]benzamide,
[40] N-(6-chlorobenzo[d]isoxazol-3-yl)-4-iodobenzamide,
[41] naphthalene-2-(6-chlorobenzo[d]isoxazol-3-yl)carboxamide,
[42] N-(6-chlorobenzo[d]isoxazol-3-yl)-3,4-difluorobenzamide,
[43] N-(6-bromobenzo[d]isoxazol-3-yl)-4-fluoro-2-trifluoromethylbenzamide,
[44] 2,4-dichloro-N-(6-bromobenzo[d]isoxazol-3-yl)-5-fluorobenzamide,
[45] N-(6-bromobenzo[d]isoxazol-3-yl)-3-fluoro-4-trifluoromethylbenzamide,
[46] N-(6-bromobenzo[d]isoxazol-3-yl)-3-fluoro-5-trifluoromethylbenzamide,
[47] N-(6-bromobenzo[d]isoxazol-3-yl)-2,3,4-trifluorobenzamide,
[48] N-(6-bromobenzo[d]isoxazol-3-yl)-4-propylbenzamide,
[49] N-(6-bromobenzo[d]isoxazol-3-yl)-3,4-difluorobenzamide,
[50] furan-2-(5-bromobenzo[d]isoxazol-3-yl)carboxamide,
[51] 7-fluoro-N-(4-fluorobenzo[d]isoxazol-3-yl)-2-trifluoromethyl benzamide,
[52] 2,4-dichloro-5-fluoro-N-(7-fluorobenzo[d]isoxazol-3-yl)benzamide,
[53] 3-fluoro-N-(7-fluorobenzo[d]isoxazol-3-yl)-4-trifluoromethylbenzamide,
[54] 2,3,4-trifluoro-N-(7-fluorobenzo[d]isoxazol-3-yl)benzamide,
[55] N-(7-fluorobenzo[d]isoxazol-3-yl)-4-iodobenzamide,
[56] 2-chloro-N-(7-fluorobenzo[d]isoxazol-3-yl)nicotinamide,
[57] naphthalene-2-(7-fluorobenzo[d]isoxazol-3-yl)carboxamide,
[58] N-(4-fluorobenzo[d]isoxazol-3-yl)-74-propylbenzamide,
[59] 3,4-difluoro-N-(7-fluorobenzo[d]isoxazol-3-yl)benzamide,
[60] 2-fluoro-N-(7-fluorobenzo[d]isoxazol-3-yl)-3-trifluoromethyl benzamide,
[61] N-(7-fluorobenzo[d]isoxazol-3-yl)-3-methoxybenzamide,
[62] N-(7-fluorobenzo[d]isoxazol-3-yl)-2,2-diphenylacetamide,
[63]furan-2-(7-fluorobenzo[d]isoxazol-3-yl)carboxamide,
[64] 2,4-dichloro-N-(4-dimethylaminobenzo[d]isoxazol-3-yl)-5-fluorobenzamide,
[65] N-(4-dimethylaminobenzo[d]isoxazol-3-yl)-2,3,4-trifluorobenzamide,
[66] 2-chloro-N-(4-dimethylaminobenzo[d]isoxazol-3-yl)nicotinamide,
[67] naphthalene-2-(4-dimethylaminobenzo[d]isoxazol-3-yl)carboxamide,
[68] N-(4-dimethylaminobenzo[d]isoxazol-3-yl)-4-propylbenzamide,
[69] N-(4-dimethylaminobenzo[d]isoxazol-3-yl)-3,4-difluorobenzamide,
[70] N-(4-dimethylaminobenzo[d]isoxazol-3-yl)-2-fluoro-3-trifluoromethylbenzamide,
[71] N-(4-dimethylaminobenzo[d]isoxazol-3-yl)-3-methoxybenzamide,
[72] N-(4-dimethylaminobenzo[d]isoxazol-3-yl)-2,2-dimethylpropionamide,
[73]cyclohexane-(4-dimethylaminobenzo[d]isoxazol-3-yl)carboxamide,
[74] N-(4-dimethylaminobenzo[d]isoxazol-3-yl)-2,2-diphenylacetamide,
[75]furan-2-(4-dimethylaminobenzo[d]isoxazol-3-yl)carboxamide,
[76] N-(4-dimethylaminobenzo[d]isoxazol-3-yl)-2,2,3,3,4,4,4-heptafluorobutyramide,
[77] 4 -fluoro-N-[4-(2,2,2-trifluoroethoxy)benzo[d]isoxazol-3-yl]-2-trifluoromethylbenzamide,
[78] 2,4-dichloro-5-fluoro-N-[4-(2,2,2-trifluoroethoxy)benzo[d]isoxazol-3-yl]benzamide,
[79] 3-fluoro-N-[4-(2,2,2-trifluoroethoxy)benzo[d]isoxazol-3-yl]-4-trifluoromethyl-benzamide,
[80] 2,3,4-trifluoro-N-[4-(2,2,2-trifluoroethoxy)benzo[d]isoxazol-3-yl]benzamide,
[81] naphthalene-2-[4-(2,2,2-trifluoroethoxy)benzo[d]isoxazol-3-yl]carboxamide,
[82] 3,4-difluoro-N-[4-(2,2,2-trifluoroethoxy)benzo[d]isoxazol-3-yl]benzamide,
[83] 2-fluoro-N-[4-(2,2,2-trifluoroethoxy)benzo[d]isoxazol-3-yl]-3-trifluoromethyl-benzamide,
[85] N-benzo[d]isoxazol-3-yl-2-ethylhexanamide,
[86] N-benzo[d]isoxazol-3-yl-2-methylbutyramide,
[88] N-benzo[d]isoxazol-3-yl-3-fluoro-4-trifluoromethylbenzamide,
[89] N-benzo[d]isoxazol-3-yl-2,3,6-trifluorobenzamide,
[90] N-benzo[d]isoxazol-3-ylnicotinamide,
[91] 5-methyl isoxazole-3-benzo[d]isoxazol-3-ylcarboxamide,
[92] benzo[b]thiophene-3-benzo[d]isoxazol-3-ylcarboxamide,
[94] 4-tert-butyl-N-(7-fluorobenzo[d]isoxazol-3-yl)benzamide,
[96] 4-fluoro-N-(7-fluorobenzo[d]isoxazol-3-yl)benzamide,
[100] 3,5-difluoro-N-(6-fluorobenzo[d]isoxazol-3-yl)benzamide,
[101] naphthalene-1-(6-fluorobenzo[d]isoxazol-3-yl)carboxamide,
[102] adamantane-1-(6-fluorobenzo[d]isoxazol-3-yl)carboxamide,
[104] 4-tert-butyl-N-(5-fluorobenzo[d]isoxazol-3-yl)benzamide,
[105] 2,4-difluoro-N-(5-fluorobenzo[d]isoxazol-3-yl)benzamide,
[106] naphthalene-2-(5-fluorobenzo[d]isoxazol-3-yl)carboxamide,
[107] N-(5-fluorobenzo[d]isoxazol-3-yl)-4-propylbenzamide,
[109] 4-tert-butyl-N-(6-chlorobenzo[d]isoxazol-3-yl)benzamide,
[110] N-(6-chlorobenzo[d]isoxazol-3-yl)-4-fluoro-2-trifluoromethylbenzamide,
[111] 2,4-dichloro-N-(6-chlorobenzo[d]isoxazol-3-yl)-5-fluorobenzamide,
[112] N-(6-chlorobenzo[d]isoxazol-3-yl)-2-ethylhexanamide,
[113] N-(6-chlorobenzo[d]isoxazol-3-yl)-2-methylbutyramide,
[115] N-(6-bromobenzo[d]isoxazol-3-yl)-3,4-dichlorobenzamide,
[116] thiophene-2-(6-bromobenzo[d]isoxazol-3-yl)carboxamide,
[117] N-(6-bromobenzo[d]isoxazol-3-yl)-3,3-dimethylbutyramide,
[119] 4-bromo-N-(5-methylbenzo[d]isoxazol-3-yl)benzamide,
[121] 4-fluoro-N-(5-methyl benzo[d]isoxazol-3-yl)benzamide,
[123] 3-methyl-N-(5-methylbenzo[d]isoxazol-3-yl)benzamide,
[124] 4-tert-butyl-N-(5-methylbenzo[d]isoxazol-3-yl)benzamide,
[126] 2,3-difluoro-N-(5-methyl benzo[d]isoxazol-3-yl)benzamide,
[127] 2,4-difluoro-N-(5-methylbenzo[d]isoxazol-3-yl)benzamide,
[128] 2-chloro-N-(5-methyl benzo[d]isoxazol-3-yl)nicotinamide,
[129] cyclopropane-(5-fluorobenzo[d]isoxazol-3-yl)carboxamide,
[130] N-(5-fluorobenzo[d]isoxazol-3-yl)-3,3-dimethylbutyramide,
[131] 2,4-difluoro-N-(5-fluorobenzo[d]isoxazol-3-yl)benzamide,
[132] 2,5-dimethylfuran-3-(5-fluorobenzo[d]isoxazol-3-yl)carboxamide,
[133] N-(5-bromobenzo[d]isoxazol-3-yl)-2,3-difluorobenzamide,
[134] 2,5-dimethylfuran-3-(5-bromobenzo[d]isoxazol-3-yl)carboxamide,
[135] N-(5-bromobenzo[d]isoxazol-3-yl)-2-methylbutyramide,
[137] N-(5-bromobenzo[d]isoxazol-3-yl)-3,4-dimethoxybenzamide,
[138] N-(5-bromobenzo[d]isoxazol-3-yl)-4-methylbenzamide,
[141] N-(5-bromobenzo[d]isoxazol-3-yl)-5-fluoro-2-trifluoromethylbenzamide,
[142] N-(5-bromobenzo[d]isoxazol-3-yl)-3-methoxybenzamide,
[143] 3-methyl-N-(5-methylbenzo[d]isoxazol-3-yl)benzamide,
[144] 4-cyano-N-(5-methylbenzo[d]isoxazol-3-yl)benzamide,
[145] N-(5-methylbenzo[d]isoxazol-3-yl)-2-phenylbutyramide,
[146] N-(5-methylbenzo[d]isoxazol-3-yl)-2-methylpentanamide,
[148] 4-chloro-N-(4-fluorobenzo[d]isoxazol-3-yl)benzamide,
[149] 2-fluoro-N-(4-fluorobenzo[d]isoxazol-3-yl)benzamide,
[150] adamantane-1-(4-fluorobenzo[d]isoxazol-3-yl)carboxamide,
[151] adamantane-1-(4-chlorobenzo[d]isoxazol-3-yl)carboxamide,
[152] N-(4-chlorobenzo[d]isoxazol-3-yl)benzamide,
[153] N-(4-chlorobenzo[d]isoxazol-3-yl)-3-methylbenzamide,
[154] N-(4-chlorobenzo[d]isoxazol-3-yl)-2-methylbutyramide,
[155] N-(4-chlorobenzo[d]isoxazol-3-yl)-3,3-dimethyl butyramide,
[156] N-(4-methoxybenzo[d]isoxazol-3-yl)-2-methylbenzamide,
[157] 2-chloro-N-(4-methoxybenzo[d]isoxazol-3-yl)benzamide,
[158] N-(4-methoxybenzo[d]isoxazol-3-yl)-2-trifluoromethylbenzamide,
[159] N-4-methoxybenzo[d]isoxazol-3-yl)-2-ethyl hexanamide,
[160] N-(4-methoxybenzo[d]isoxazol-3-yl)-2-methylbutyramide,
[161] N-(4-methoxybenzo[d]isoxazol-3-yl)-2-methylpentanamide,
[162]cyclopropane-[4-(2,2,2-trifluoroethoxy)benzo[d]isoxazol-3-yl]carboxamide,
[163] 2-methyl-N-[4-(2,2,2-trifluoroethoxy)benzo[d]isoxazol-3-yl]butyramide,
[164] 3,3-dimethyl-N-[4-(2,2,2-trifluoroethoxy)benzo[d]isoxazol-3-yl]butyramide,
[165] cyclohexane-[4-(2,2,2-trifluoroethoxy)benzo[d]isoxazol-3-yl]carboxamide,
[166] 2,5-dimethylfu ran-3-[4-(2,2,2-trifluoroethoxy)benzo[d]isoxazol-3-yl]carboxamide,
[167] N-[4-(4-methylbenzyloxy)benzo[d]isoxazol-3-yl]-3-nitrobenzamide,
[168] N-[4-(4-methylbenzyloxy)benzo[d]isoxazol-3-yl]-4-propylbenzamide,
[169] N-(4-dimethylaminobenzo[d]isoxazol-3-yl)-3-fluoro-5-trifluoromethylbenzamide,
[170] 3,4-dichloro-N-(4-dimethylaminobenzo[d]isoxazol-3-yl)benzamide,
[171] N-(4-dimethylaminobenzo[d]isoxazol-3-yl)-3-nitrobenzamide,
[172] adamantane-1-(4-dimethylaminobenzo[d]isoxazol-3-yl)carboxamide,
[173] benzo[b]thiophene-2-(4-dimethylaminobenzo[d]isoxazol-3-yl)carboxamide,
[174] N-(4-dimethylaminobenzo[d]isoxazol-3-yl)-3,3-dimethylbutyramide,
[176] N-(6-amino-4,5,7-trifluorobenzo[d]isoxazol-3-yl)-2,6-difluorobenzamide
   and
[177] N-(6-amino-4,5,7-trifluorobenzo[d]isoxazol-3-yl)-2,4-difluorobenzamide.

The present invention further relates to a process for preparing compounds of the general formula Ia indicated above, in which at least one compound of general formula IIa

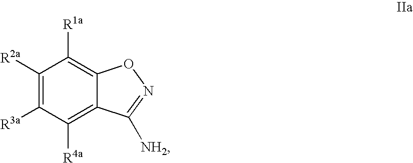

in which R^{1a} to R^{4a} have the aforementioned meaning, is reacted
in a reaction medium, where appropriate in the presence of at least one base, with at least one compound of the general formula R^{5a}—C(═O)—X in which R^{5a} has the aforementioned meaning, and X is a leaving group, preferably a halogen radical, particularly preferably a chlorine atom,
or in a reaction medium in the presence of at least one coupling reagent, where appropriate in the presence of at least one base, with at least one compound of the general formula R^{5a}—C(═O)—OH in which R^{5a} has the aforementioned meaning, to give a compound of the general formula Ia,

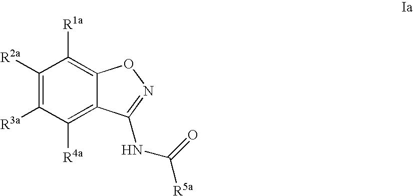

in which R^{1a} to R^{5a} have the aforementioned meaning, and the latter is isolated and/or purified where appropriate.

The process of the invention for preparing substituted N-benzo[d]isoxazol-3-ylamine derivatives of the invention, of the general formula Ia indicated above, is also indicated in scheme 1 below. The method of the invention is likewise suitable for preparing substituted N-benzo[d]isoxazol-3-ylamine derivatives of the invention, of the general formula I indicated above.

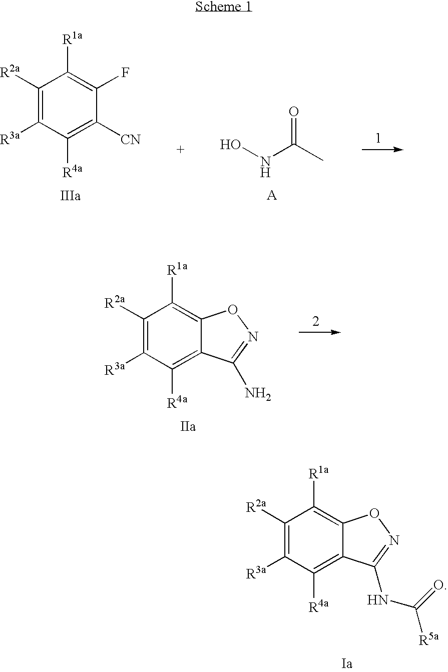

In stage 1, substituted 2-fluorobenzonitriles of the general formula IIIa in which R^{1a} to R^{4a} have the aforementioned meaning are reacted in a reaction medium, preferably selected from the group consisting of diethyl ether, tetrahydrofuran, acetonitrile, dimethyl sulfoxide, dimethylformamide and dichloromethane, in the presence of at least one base, preferably in the presence of at least one alkali metal alcoholate salt, particularly preferably in the presence of an alkali metal alcoholate salt selected from the group consisting of potassium methanolate, sodium methanolate, potassium tert-butoxide and sodium tert-butoxide, with acetohydroxamic acid (A), preferably at temperatures of from 20° C. to 100° C., to give compounds of the general formula IIa. In stage 2, compounds of the general formula IIa indicated above are reacted with carboxylic acids of the general formula R^{5a}—C(═O)—OH in which R^{5a} has the aforementioned meaning, in a reaction medium, preferably selected from the group consisting of diethyl ether, tetrahydrofuran, acetonitrile, dimethylformamide and dichloromethane, where appropriate in the presence of at least one coupling reagent, preferably selected from the group consisting of 1-benzotriazolyloxy-tris(dimethyl-amino)phosphonium hexafluorophosphate (BOP), dicyclohexylcarbodiimide (DCC), N′-(3-dimethylaminopropyl)-N-ethylcarbodiimide (EDCI), N-[(dimethylamino)-1H-1,2,3-triazolo[4,5-b]pyridino-1-ylmethylene]-N-methylmethanaminium hexafluorophosphate N-oxide (HATU), O-(benzotriazol-1-yl)-N,N,N′,N′-tetramethyluroniom hexafluorophosphate (HBTU) and 1-hydroxy-7-azabenzotriazole (HOAt), where appropriate in the presence of at least one inorganic base, preferably selected from the group consisting of potassium carbonate and cesium carbonate, or of an organic base, preferably selected from the group consisting of triethylamine, pyridine, dimethylaminopyridine and diisopropylethylamine, preferably at temperatures of from −70° C. to 100° C., to give compounds of the general formula Ia.

Alternatively, compounds of the general formula IIa are reacted with carboxylic acid derivatives or carbonic acid derivatives of the general formula R^{5a}—C(═O)—X where X is a halogen radical, preferably chlorine or bromine, in a reaction medium, preferably selected from the group consisting of diethyl ether, pyridine, tetrahydrofuran, acetonitrile, dimethylformamide and dichloromethane, preferably in the presence of at least one organic or inorganic base, for example triethylamine, dimethylamino-pyridine, pyridine, diisopropylamine, alkali metal hydroxides, alkali metal carbonates, alkaline earth metal hydroxides and alkaline earth metal carbonates, particularly preferably in the presence of an organic base selected from the group consisting of triethylamine, dimethylaminopyridine, pyridine and diisopropylamine, at temperatures of preferably from −70° C. to 100° C., to give compounds of the general formula Ia.

The reactions described above can in each case be carried out under usual conditions familiar to the skilled worker, for example in relation to pressure or sequence or addition of the components. It is possible where appropriate for the skilled worker to ascertain the optimal management of the process under the respective conditions by simple preliminary tests.

The compounds of the formulae IIIa indicated above and of the general formulae R^{5a}—C(═O)—OH and R^{5a}—C(═O)—X can in each case be purchased commercially and/or can be prepared by usual processes known to the skilled worker.

The intermediates and final products obtained after the reactions described above can in each case, if desired and/or necessary, be purified and/or isolated by usual methods known to the skilled worker. Suitable purification methods are for example extraction methods and chromatographic methods such as column chromatography or preparative chromatography.

All of the process steps described above, and in each case also the purification and/or isolation of intermediates and final products, can be carried out partly or completely under an inert gas atmosphere, preferably under nitrogen atmosphere or argon atmosphere.

The substituted N-benzo[d]isoxazol-3-ylamine derivatives of the invention, of the aforementioned general formulae I and Ia, and corresponding stereoisomers can be isolated both in the form of their free bases, their free acids and in the form of corresponding salts, especially physiologically tolerated salts.

The free bases of the respective substituted N-benzo[d]isoxazol-3-ylamine derivatives of the invention, of the aforementioned general formulae I and Ia, and corresponding stereoisomers can be converted for example by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid, into the corresponding salts, preferably physiologically tolerated salts.

The free bases of the respective substituted N-benzo[d]isoxazol-3-ylamine derivatives of the aforementioned general formulae I and Ia and corresponding stereoisomers can likewise be converted with the free acid or a salt of a sugar substitute, such as, for example, saccharin, cyclamate or acesulfame, into the corresponding physiologically tolerated salts.

Correspondingly, the free acids of the substituted N-benzo[d]isoxazol-3-ylamine derivatives of the aforementioned general formulae I and Ia and corresponding stereoisomers can be converted by reaction with a suitable base into the corresponding physiologically tolerated salts. Examples which may be mentioned are the alkali metal salts, alkaline earth metal salts or ammonium salts [NHₓR₄₋ₓ]⁺, in which x is 0, 1, 2, 3 or 4 and R is a linear or branched C₁₋₄-alkyl radical.

The substituted N-benzo[d]isoxazol-3-ylamine derivatives of the invention, of the aforementioned general formulae I and Ia, and corresponding stereoisomers can also where appropriate, just like the corresponding acids, the corresponding bases or salts of these compounds, be obtained in the form of their solvates, preferably in the form of their hydrates, by usual methods known to the skilled worker.

Where the substituted N-benzo[d]isoxazol-3-ylamine derivatives of the invention, of the aforementioned general formulae I and Ia, are obtained after their preparation in the form of a mixture of their stereoisomers, preferably in the form of their racemates or other mixtures of their various enantiomers and/or diastereomers, these can be separated and, where appropriate, isolated by usual methods known to the skilled worker. Examples which may be mentioned are chromatographic separation methods, in particular liquid chromatographic methods under atmospheric pressure or under elevated pressure, preferably MPLC and HPLC methods, and methods of fractional crystallization. It is possible in this connection in particular for individual enantiomers to be separated from one another for example by means of HPLC on a chiral stationary phase or by means of crystallization of diastereomeric salts formed with chiral acids, for instance (+)-tartaric acid, (−)-tartaric acid or (+)-10-camphor sulfonic acid.

The present invention further relates to a medicament comprising at least one compound of the invention of the general formula Ia, where appropriate in the form of one of its pure stereoisomers, in particular enantiomers or diastereomers or rotamers, its racemate or in the form of mixtures of stereoisomers, in particular of enantiomers or diastereomers, in any mixing ratio, or where appropriate in the form of a corresponding salt or in each case in the form of a corresponding solvate, and where appropriate one or more physiologically tolerated excipients.

The medicaments of the invention are particularly suitable for mGluR5 receptor regulation, preferably for inhibiting the mGluR5 receptor, and/or for noradrenaline receptor regulation, preferably for noradrenaline reuptake inhibition, and/or for serotonin (5-HT) receptor regulation, preferably for serotonin reuptake inhibition, and thus also for the prophylaxis and/or treatment of disorders and diseases which are mediated at least partly by mGluR5 receptors and/or noradrenaline receptors and/or serotonin receptors.

The medicament of the invention is preferably suitable for the treatment and/or prophylaxis of one or more disorders selected from the group consisting of disorders of food intake, preferably selected from the group consisting of bulimia, anorexia, obesity and cachexia;

pain, preferably pain selected from the group consisting of acute pain, chronic pain, visceral pain and neuropathic pain; migraines; chronic paroxysmal hemicrania; depression; urinary incontinence; cough, asthma; glaucoma; tinitus; inflammations; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); epilepsy; narcolepsy; diarrhea; gastritis, gastric ulcer; pruritus; anxiety states; panic attacks; schizophrenia; cerebral ischemia; muscle spasms; cramps; gastroesaphageal reflux syndrome; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency;
for the prophylaxis and/or reduction of a development of tolerance to medicaments and/or drugs, especially medicaments based on opioids; for regulating food intake; for modulating motor activity, for regulating the cardiovascular system; for local anesthesia; for increasing vigilance; for increasing libido; for diuresis and/or for antinatriuresis.

The medicament of the invention is particularly preferably suitable for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraine; depression; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); anxiety states; panic attacks; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency.

The medicament of the invention is very particularly preferably suitable for the treatment and/or prophylaxis of pain, preferably pain selected from the group consisting of acute pain, chronic pain, visceral pain and neuropathic pain.

The present invention further relates to the use of at least one substituted N-benzo[d]isoxazol-3-ylamine derivative of the invention, of the general formula Ia indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for the manufacture of a medicament for mGluR5 receptor regulation, preferably for inhibiting the mGluR5 receptor, and/or for noradrenaline receptor regulation, preferably for noradrenaline reuptake inhibition, and/or for serotonin (5-HT) receptor regulation, preferably for serotonin reuptake inhibition.

Preference is given to the use of at least one substituted N-benzo[d]isoxazol-3-yl-amine derivative of the invention, of the general formula Ia indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the prophylaxis and/or treatment of disorders and diseases which are mediated at least partly by mGluR5 receptors and/or noradrenaline receptors and/or serotonin receptors.

Particular preference is given to the use of at least one substituted N-benzo[d]isoxazol-3-ylamine derivative of the invention, of the general formula Ia indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraines; chronic paroxysmal hemicrania; depression; urinary incontinence; cough; asthma; glaucoma; tinitus; inflammations; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); epilepsy; narcolepsy; diarrhea; gastritis, gastric ulcer; pruritus; anxiety states; panic attacks; schizophrenia; cerebral ischemia; muscle spasms; cramps; gastroesaphageal reflux syndrome; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency; for the prophylaxis and/or reduction of a development of tolerance to medicaments and/or drugs, especially medicaments based on opioids; for regulating food intake; for modulating motor activity, for regulating the cardiovascular system; for local anesthesia; for increasing vigilance; for increasing libido; for diuresis and/or for antinatriuresis.

Very particular preference is given to the use of at least one substituted N-benzo[d]isoxazol-3-ylamine derivative of the invention, of the general formula Ia indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of one or more disorders selected from the group consisting of disorders of food intake, preferably selected from the group consisting of bulimia, anorexia, obesity and cachexia; pain, preferably pain selected from the group consisting of acute pain, chronic pain, visceral pain and neuropathic pain; migraine; depression; neurodegenerative disorders, preferably selected from the group consisting of Parkinson's disease, Huntington's disease, Alzheimer's disease and multiple sclerosis; cognitive dysfunctions, preferably memory impairments; cognitive deficiencies (attention deficit syndrome, ADS); anxiety states; panic attacks; alcohol and/or drug abuse, preferably nicotine or cocaine, and/or medicament abuse; alcohol and/or drug dependency, preferably nicotine or cocaine, and/or medicament dependency, preferably for the prophylaxis and/or reduction of withdrawal manifestations associated with alcohol and/or drug dependency, preferably nicotine or cocaine, and/or medicament dependency.

Yet more preference is given to the use of at least one substituted N-benzo[d]isoxazol-3-ylamine derivative of the invention, of the general formula Ia indicated above, in each case optionally in the form of one of its pure stereoisomers, in particular enantiomers or diastereomers or rotamers, its racemates or in the form of a mixture of stereoisomers, in particular of enantiomers and/or diastereomers and/or rotamers, in any mixing ratio, or in each case in the form of a corresponding salt, or in each case in the form of a corresponding solvate, and where appropriate one or more pharmaceutically acceptable excipients for manufacturing a medicament for the treatment and/or prophylaxis of pain, preferably of pain selected from the group consisting of acute pain, chronic pain, visceral pain and neuropathic pain.

The medicament of the invention is suitable for administration to adults and children, including infants and babies.

The medicament of the invention can be in the form of a liquid, semisolid or solid pharmaceutical form, for example in the form of solutions for injection, drops, elixiers, syrups, sprays, suspensions, tablets, patches, capsules, plasters, suppositories, ointments, creams, lotions, gels, emulsions, aerosols or in multiparticulate form, for example in the form of pellets or granules, where appropriate compressed to tablets, packed into capsules or suspended in a liquid, and be administered as such. Besides at least one substituted N-benzo[d]isoxazol-3-ylamine derivative of the general formula Ia indicated above, where appropriate in the form of one of its pure stereoisomers, in particular enantiomers or diastereomers or rotamers, its racemate or in the form of mixtures of stereoisomers, in particular of enantiomers or diastereomers or rotamers, in any mixing ratio, or where appropriate in the form of a corresponding salt or in each case in the form of a corresponding solvate, the medicament of the invention normally comprises further physiologically tolerated pharmaceutical excipients which can preferably be selected from the group consisting of carrier materials, fillers, solvates, diluents, surface-active substances, colorants, preservatives, disintegrants, glidants, lubricants, flavorings and binders.

The selection of the physiologically tolerated excipients and the amounts thereof to be employed depends on whether the medicament is to be administered orally, subcutaneously, parenterally, intravenously, intraperitoneally, intradermally, intramuscularly, intranasally, buccally, rectally or locally, for example on infections of the skin, the mucous membranes and of the eyes. Suitable and preferred for oral administration are preparations in the form of tablets, coated tablets, capsules, granules, pellets, drops, elixirs and syrups, and for parenteral, topical and inhalational administration are solutions, suspensions, easily reconstitutable dry preparations, and sprays.

The substituted N-benzo[d]isoxazol-3-ylamine derivatives of the invention, of the general formula Ia indicated above, which are employed in the medicament of the invention may be in the form of a depot, in dissolved form or in a plaster, where appropriate with the addition of agents promoting skin penetration, as suitable percutaneous administration preparations. Formulations which can be used orally or percutaneously may also release the particular substituted N-benzo[d]isoxazol-3-ylamine derivatives of the invention, of the general formula Ia indicated above, in a delayed manner. It is possible in principle to add to the medicament of the invention other further active ingredients known to the skilled worker.

The medicaments of the invention are manufactured with the aid of conventional means, apparatuses, methods and processes known from the prior art, as described for example in “Remington's Pharmaceutical Sciences”, edited by A. R. Gennaro, 17^{th} edition, Mack Publishing Company, Easton, Pa., 1985, especially in part 8, chapter 76 to 93. The corresponding description is introduced hereby as reference and forms part of the disclosure.

The amount of the particular substituted N-benzo[d]isoxazol-3-ylamine derivatives of the invention, of the general formula Ia indicated above, to be administered to the patient may vary and depends for example on the weight or age of the patient and on the mode of administration, the indication and the severity of the disorder. Normally, 0.005 to 100 mg/kg, preferably 0.05 to 75 mg/kg, of the patient's body weight of at least one such compound of the invention are administered.

### Pharmacological Methods

### I. Method for Determining the Noradrenaline and 5HT Uptake Inhibition:

For in vitro studies, synaptosomes were freshly isolated from rat brain areas as described in the publication “The isolation of nerve endings from brain” by E. G. Gray and V. P. Whittaker, J. Anatomy 96, pages 79-88, 1962. The corresponding literature description is introduced hereby as reference and forms part of the disclosure.

The tissue (hypothalamus for determining the noradrenaline uptake inhibition and medulla and pons for determining the 5HT uptake inhibition) was homogenized in ice-cold 0.32 M sucrose (100 mg of tissue/1 ml) in a glass homogenizer with Teflon pestle using five full up and down strokes at 840 revolutions/minute. The homogenate was centrifuged at 1000 g and at 4° C. for 10 minutes. After subsequent centrifugation at 17 000 g for 55 minutes, the synaptosomes (P₂ fraction) are obtained and were resuspended in 0.32 M glucose (0.5 ml/100 mg of the original weight).

The respective uptake was measured in a 96-well microtiter plate. The volume was 250 μl and incubation took place at room temperature (approx. 20-25° C.) under an O₂ atmosphere.

The incubation time was 7.5 minutes for [³H]-NA and 5 minutes for [³H]-5-HT. The 96 samples were then filtered through a Unifilter GF/B® microtiter plate (Packard) and washed with 200 ml of incubated buffer using a “Brabdel MPXRI-96T Cell Harvester”. The Unifilter GF/B plate was dried at 55° C. for 1 h. The plate was then sealed with a Back Seal® (Packard) and 35 μl of scintillation fluid were added per well (Ultima Gold®, Packard). After sealing with a top Seal® (Packard), the radioactivity was determined, after equilibrium had been reached (about 5 h), in a “Trilux 1450 Microbeta” (Wallac).

The following characteristic data were found for the NA transporter:

NA uptake: Km=0.32±0.11 μM

The amount of protein employed in the above determination corresponded to the value disclosed in the literature, as described for example in “Protein measurement with the folin phenol reagent”, Lowry et al., J. Biol. Chem., 193, 265-275, 1951. A detailed description of the method can also be found in the literature, for example from M. Ch. Frink, H.-H. Hennies, W. Engelberger, M. Haurand and B. Wilffert (1996) Arzneim.-Forsch./Drug Res. 46 (III), 11, 1029-1036. The corresponding literature descriptions are hereby introduced as reference and form part of the disclosure.

II. Method for Determining the Affinity for the mGluR5 Receptor

Pig brain homogenate is prepared by homogenizing (Polytron PT 3000, Kinematica AG, 10 000 revolutions per minute for 90 seconds) pig brain hemispheres without medulla, cerebellum and pons in buffer of pH 8.0 (30 mM hepes, Sigma, order No. H3375+1 tablet of complete for 100 ml, Roche Diagnostics, order No. 1836145) in the ratio 1:20 (brain weight/volume) and differential centrifugation at 900×g and 40 000×g. In each case 450 μg of protein from brain homogenate are incubated with 5 nM³[H]-MPEP (Tocris, order No. R1212) (MPEP=2-methyl-6-(3-methoxyphenyl)-ethynylpyridine) and the compounds to be investigated (10 μM in the assay) in buffer (as above) in 250 μl incubation mixtures in 96-well microtiter plates at room temperature for 60 min.

The mixtures are then filtered with the aid of a Brandel Cell Harvester (Brandel, TYP Robotic 9600) on unifilter plates with glass fiber filter mats (Perkin Elmer, order No. 6005177) and subsequently washed with buffer (as above) 3 times with 250 μl per sample each time. The filter plates were then dried at 55° C. for 60 min. 30 μl of Ultima Gold™ scintillator (Packard BioScience, order No. 6013159) are then added to each well and, after 3 hours, the samples are measured in a β counter (Mikrobeta, Perkin Elmer). The nonspecific binding is determined by adding 10 μM MPEP (Tocris, order No. 1212).

The invention is explained by means of examples below. These explanations are merely by way of example and do not restrict the general concept of the invention.

### EXAMPLES

The yields of the prepared compounds are not optimized.

All temperatures are uncorrected.

### Abbreviations

aq. aqueous
APCI atmospheric pressure chemical ionisation
DCM dichloromethane
DMF dimethylformamide
DMSO dimethyl sulfoxide
EtOAc ethyl acetate
h hours
min minutes
NMR nuclear magnetic resonance spectroscopy
RT room temperature
sat. saturated

The chemicals and solvents employed were purchased commercially from conventional suppliers (Acros, Avocado, Aldrich, Bachem, Fluka, Lancaster, Maybridge, Merck, Sigma, TCI, etc.) or synthesized by methods known to the skilled worker.

The stationary phase employed for the column chromathography was silica gel 60 (0.040-0.063 mm) supplied by E. Merck, Darmstadt.

The thin-layer chromatographic investigations were carried out with HPTLC precoated plates, silica gel 60 F 254, supplied by E. Merck, Darmstadt.

The mixing ratios of solvents, mobile phases or for chromatographic investigations are always stated in volume/volume.

Analyses took place by mass spectroscopy and NMR.

General Methods for Preparing Exemplary Substituted N-benzo[d]isoxazol-3-ylamine Derivatives

The substituted N-benzo[d]isoxazol-3-ylamine derivatives of the invention, of the general formula Ia, can be obtained from substituted 2-fluorobenzonitriles of the general formula IIIa in two stages as depicted in scheme 2.

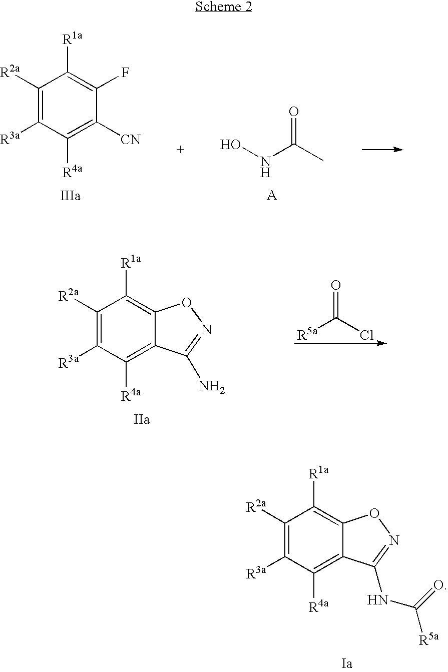

1. General Method for Preparing Substituted benzo[d]isoxazol-3-ylamines of the General Formula IIa

Acetohydroxamic acid (A) (1.1 equivalents) was suspended under an inert gas atmosphere in DMF (1.45 ml per mmol of the compound of the general formula IIIa). Potassium tert-butoxide (1.1 equivalents) were added and the reaction mixture was stirred at RT for 30 min, the compound of the general formula IIIa (1 equivalent) was added, and the mixture was stirred at 50° C. for 1 h. After cooling, the reaction mixture was added to a mixture of sat. aq. NaCl solution (0.9 ml per mmol of A) and EtOAc (0.9 ml per mmol of A) and stirred for 30 min. The phases were separated and the aqueous phase was extracted several times with EtOAc (in each case 0.8 ml per mmol of A). The combined organic phases were washed several times with sat. aq. NaCl solution (in each case 0.8 ml per mmol of A), dried over MgSO₄ and the solvent was removed in vacuo.

If it was necessary to form the corresponding hydrochloride for purification, the residue was taken up in each case in methyl ethyl ketone (8.7 ml per g of residue). After addition of water (0.1 ml per g of residue), trimethylchlorosilane (0.7 ml per g of residue) was added dropwise while stirring slowly and cooling in ice. The reaction mixture was cooled to 4° C. for crystallization. The precipitate which separated out was filtered off and dried in a desiccator with the aid of phosphorus pentoxide as desiccant.

In this way, the following compounds of the general formula IIa were obtained where appropriate in the form of the corresponding hydrochloride or dihydrochloride.
[A1] Benzo[d]isoxazol-3-ylamine
[A2] 4-(4-Methylbenzyloxy)benzo[d]isoxazol-3-ylamine

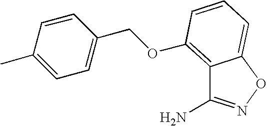

δ (DMSO, 300 MHz)=2.32 (s, 3H); 5.24 (s, 2H); 6.74 (d, 1H, J=7.9 Hz); 6.94 (d, 1H, J=8.3 Hz); 7.20 (d, 2H, J=8.3 Hz); 7.38 (dd, 3H, J=10.9 Hz, J=9.0 Hz).
[A3] 4-Methoxybenzo[d]isoxazol-3-ylamine
[A4] 4-Chlorobenzo[d]isoxazol-3-ylamine dihydrochloride

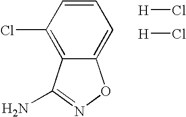

δ (DMSO, 300 MHz)=6.09 (br.s, 4H); 7.25 (dd, 1H, J=0.8 Hz, J=7.5 Hz); 7.41-7.55 (m, 2H).
[A5] 4-Fluorobenzo[d]isoxazol-3-ylamine
[A6] Benzo[d]isoxazol-3,4-diamine hydrochloride

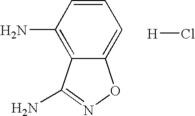

δ (DMSO, 300 MHz)=6.39 (t, 1H, J=8.7 Hz); 6.62 (d, 1H, J=8.7 Hz); 7.27 (dd, 1H, J=6.8 Hz, J=8.3 Hz); 8.10 (br.s, 3H).
[A7] N⁴,N⁴-Dimethylbenzo[d]isoxazol-3,4-diamine hydrochloride

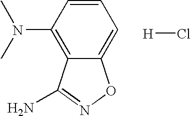

δ (DMSO, 300 MHz)=3.04 (s, 3H), 3.07 (s, 3H); 6.70 (t, 1H, J=8.7 Hz); 6.77 (d, 1H, J=8.7 Hz); 7.15 (d, 1H, J=7.9 Hz); 7.33 (d, 1H, J=8.3 Hz); 7.41-7.58 (m, 1H).
[A8] 4-(2,2,2-Trifluoroethoxy)benzo[d]isoxazol-3-ylamine

δ (DMSO, 300 MHz)=4.91-5.11 (m, 2H); 5.80 (br.s, 2H), 7.08-7.31 (m, 2H); 7.73-7.85 (m, 1H).
[A9] 5-Methylbenzo[d]isoxazol-3-ylamine
[A10] 5-Bromobenzo[d]isoxazol-3-ylamine
[A11] 5-Fluorobenzo[d]isoxazol-3-ylamine hydrochloride

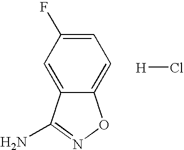

δ (DMSO, 300 MHz)=7.39 (dt, 1H, J=2.2 Hz, J=9.0 Hz); 7.49 (dd, 1H, J=4.2 Hz, J=9.0 Hz); 7.78 (dd, 1H, J=2.2 Hz, J=7.5 Hz).
[A12] 6-Fluorobenzo[d]isoxazol-3-ylamine
[A13] 6-Chlorobenzo[d]isoxazol-3-ylamine hydrochloride

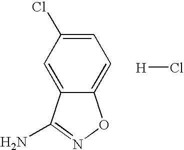

δ (DMSO, 300 MHz)=7.29 (dd, 1H, J=1.5 Hz, J=7.5 Hz); 7.59 (d, 1H, J=1.9 Hz); 7.93 (d, 1H, J=8.3 Hz); 8.73 (br.s, 1H).
[A14] 6-Bromobenzo[d]isoxazol-3-ylamine
[A15] 7-Fluorobenzo[d]isoxazol-3-ylamine

δ (DMSO, 300 MHz)=6.53 (s, 2H); 7.22 (dt, J=4.2 Hz, J=7.5 Hz); 7.37 (dd, 1H, J=10.9 Hz, J=12.0 Hz); 7.66 (d, 1H, J=7.9 Hz).
[A16] 4,5,7-Trifluorobenzo[d]isoxazol-3,6-diamine

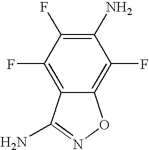

MS (APCI)=204 [M⁺+1]; 184 [M⁺+1−HF]

2. General Method for Reacting Substituted benzo[d]isoxazol-3-ylamines to Give Substituted N-benzo[d]isoxazol-3-ylamine Derivatives of the General Formula Ia
a. Manual Synthesis

The substituted benzo[d]isoxazol-3-ylamines of the general formula IIa (2 equivalents) were dissolved in pyridine (0.5 ml per mmol of benzo[d]isoxazol-3-ylamine of the general formula IIa). The reaction mixture was cooled to −15° C., the appropriate carbonyl chloride R^{5a}—C(═O)—Cl (1 equivalent) was slowly added, and the mixture was stirred overnight. For workup, the reaction mixture was poured into water (8.5 ml per mmol of benzo[d]isoxazol-3-ylamine of the general formula IIa). The resulting precipitate was filtered off with suction, washed with water, dried in vacuo and recrystallized from toluene.

The following substituted N-benzo[d]isoxazol-3-ylamine derivatives of the invention were prepared as described under 2a.

### Exemplary Compounds

1. Thiophene-2-benzo[d]isoxazol-3-ylcarboxamide

δ (DMSO, 600 MHz)=7.26 (t, 1H, J=4.5 Hz); 7.37 (t, 1H, J=7.6 Hz); 7.63-7.71 (m, 2H); 7.93 (d, 1H, J=4.5 Hz); 8.10 (d, 1H, J=7.6 Hz); 8.21 (d, 1H, J=3.0 Hz), 11.53 (s, 1H).
2. N-Benzo[d]isoxazol-3-ylsuccinic acid methyl ester

δ (DMSO, 600 MHz)=2.65-2.70 (m, 2H); 2.74-2.89 (m, 2H); 3.63 (s, 3H), 7.31-7.36 (m, 1H); 7.58-7.67 (m, 2H); 7.99-8.06 (m, 1H); 11.09 (s, 1H).
3. Naphthalene-2-benzo[d]isoxazol-3-ylcarboxamide

δ (DMSO, 600 MHz)=7.39 (t, 1H, J=7.3 Hz); 7.61-7.73 (m, 4H); 8.00-8.13 (m, 5H); 8.76 (s, 1H); 11.61 (s, 1H).
4. Adamantane-2-benzo[d]isoxazol-3-ylcarboxamide

δ (DMSO, 600 MHz)=1.71-1.75 (m, 6H); 1.97-2.02 (m, 6H); 2.04-2.07 (m, 3H); 7.32 (dd, 1H, J=6.8 Hz, J=7.5 Hz); 7.58-7.67 (m, 2H); 7.84 (dd, 1H, J=6.8 Hz, J=7.5 Hz); 10.38 (s, 1H).
5. Cyclohexanebenzo[d]isoxazol-3-ylcarboxamide

δ (DMSO, 600 MHz)=1.22-1.51 (m, 3H); 1.52-1.80 (m, 3H); 1.81-1.95 (m, 2H); 1.98-2.13 (m, 2H); 2.45-2.64 (m, 1H); 7.23-7, (m, 1H); 7.46-7.65 (m, 2H); 8.29 (d, 1H, J=8.3 Hz); 9.12 (s, 1H).
6. N-Benzo[d]isoxazol-3-yl-2,2-dimethylpropionamide

δ (DMSO, 600 MHz)=1.31 (s, 9H); 7.33 (t, 1H, 7.5 Hz); 7.59-7.65 (m, 2H); 7.83-7.86 (m, 1H); 10.47 (s, 1H).

b. Automated Synthesis

Firstly, the following stock solutions were produced:
Solution I: 0.1 M solution of the benzo[d]isoxazol-3-ylamine of the general formula IIa in pyridine
Solution II: 0.5 M solution of the carbonyl chloride of the general formula R^{5a}—C(═O)—Cl in pyridine
Solution III: 0.1 M solution of triethylamine in pyridine

Solution I (1 ml) was introduced into a dry screw-cap tube with septum cap at RT, and solution II (1 ml) and solution III (1 ml) were added. The reaction mixture was stirred in a hit reactor (supplied by Zymark, Rüsselsheim, Germany) at RT for 24 h and, in the quench station (supplied by Zymark, Rüsselsheim, Germany), 1 M hydrochloric acid solution (2 ml) was added at RT so that a pH of 3 was reached. After 30 min, DCM (2 ml) was added by pipette in the transfer block, and the reaction mixture was mixed in the spin reactor for 30 min. The stirrer bar was removed by filtration and the vessel was rinsed with DCM (2 ml).

The organic phase was removed and collected. The aqueous phase was mixed with DCM (1.5 ml), treated in a vortexer and vigorously mixed in the spin reactor for 15 min. After centrifugation, the organic phase was separated off and combined with the first organic phase. The aqueous phase is extracted analogously with DCM a second time. The combined organic phases were then dried over a magnesium sulfate cartridge, and the solvent was removed in a GeneVac HT series 1 evaporator system (GeneVac, Wiesbaden, Germany).

The following substituted N-benzo[d]isoxazol-3-ylamine derivatives of the invention were prepared as described under 2b.

### Exemplary Compounds

7. N-(4-methoxybenzo[d]isoxazol-3-yl)-2,2-diphenylacetamide,

MS (APCI)=359 [M⁺+1]
8. cyclopropane(5-bromobenzo[d]isoxazol-3-yl)carboxamide,

MS (APCI)=281 [M (79Br)⁺+1]; 283 [M (⁸¹Br)⁺+1], 213.
9. 3,5-dichloro-N-(6-fluorobenzo[d]isoxazol-3-yl)benzamide,

MS (APCI)=325 [M(³⁵Cl³⁵Cl)⁺+1]; 327 [M(³⁵Cl³⁷Cl)⁺+1]; 329 [M(³⁷Cl³⁷Cl)⁺+1]
10. N-(4-aminobenzo[d]isoxazol-3-yl)-4-nitrobenzamide,

MS (APCI)=299 [M⁺+1], 150
11. naphthalene-1-(4-aminobenzo[d]isoxazol-3-yl)carboxamide,

MS (APCI)=304 [M⁺+1], 155.
12. benzo[b]thiophene-2-(4-fluorobenzo[d]isoxazol-3-yl)carboxamide,

MS (APCI)=313 [M⁺+1], 161.
13. N-benzo[d]isoxazol-3-yl-2-trifluoromethylbenzamide,

MS (APCI)=307 [M⁺+1]; 287.
14. N-benzo[d]isoxazol-3-yl-3,4-dichlorobenzamide

MS (APCI)=307 [M(³⁵Cl³⁵Cl)⁺+1]; 309 [M(³⁵Cl³⁷Cl)⁺+1]; 311 [M(³⁷Cl³⁷Cl)⁺+1]
15. N-benzo[d]isoxazol-3-yl-2,3-difluorobenzamide,

MS (APCI)=275 [M⁺+1]
16. N-(4-dimethylaminobenzo[d]isoxazol-3-yl)-3-fluorobenzamide,

MS (APCI)=300 [M⁺+1]
17. thiophene-2-(6-fluorobenzo[d]isoxazol-3-yl)carboxamide,

MS (APCI)=263 [M⁺+1], 153.
18. N-(6-fluorobenzo[d]isoxazol-3-yl)-2-methylbutyramide,

MS (APCI)=237 [M⁺+1], 153.
19. N-(6-chlorobenzo[d]isoxazol-3-yl)-2-fluoro-3-trifluoromethylbenzamide,

MS (APCI)=359 [M (³⁵Cl)⁺+1]; 361[M (³⁷Cl)⁺+1].
20. N-(6-chlorobenzo[d]isoxazol-3-yl)-3-methoxybenzamide,

MS (APCI)=303 [M (³⁵Cl)⁺+1]; 305 [M (³⁷Cl)⁺+1], 135.
21. N-(6-bromobenzo[d]isoxazol-3-yl)-4-tert-butylbenzamide,

MS (APCI)=373 [M (⁷⁹Br)⁺+1]; 375 [M (⁸¹Br)⁺+1].
22. N-(6-bromobenzo[d]isoxazol-3-yl)-2-methoxybenzamide,

MS (APCI)=347 [M (⁷⁹Br)⁺+1]; 349 [M (⁸¹Br)⁺+1].
23. N-(6-bromobenzo[d]isoxazol-3-yl)-2-trifluoromethyl benzamide,

MS (APCI)=385 [M (⁷⁹Br)⁺+1]; 387 [M (⁸¹Br)⁺+1].
24. adamantane-2-(6-bromobenzo[d]isoxazol-3-yl)carboxamide,

MS (APCI)=375 [M (⁷⁹Br)⁺+1]; 377 [M (⁸¹Br)⁺+1]
25. N-(6-bromobenzo[d]isoxazol-3-yl)-2-methylbutyramide,

MS (APCI)=297 [M (⁷⁹Br)⁺+1]; 299 [M (⁸¹Br)⁺+1], 213.
26. N-(5-fluorobenzo[d]isoxazol-3-yl)-2-trifluoromethylbenzamide,

MS (APCI)=325 [M⁺+1]; 305.
27. 3,4-dichloro-N-(5-fluorobenzo[d]isoxazol-3-yl)benzamide,

MS (APCI)=325 [M(³⁵Cl³⁵Cl)⁺+1]; 327 [M(³⁵Cl³⁷Cl)⁺+1]; 329 [M(³⁷Cl³⁷Cl)⁺+1]
28. N-(5-fluorobenzo[d]isoxazol-3-yl)-2-methylbenzamide,

MS (APCI)=271 [M⁺+1]; 119.
29. N-(5-bromobenzo[d]isoxazol-3-yl)-2-fluoro-5-trifluoromethylbenzamide,

MS (APCI)=403 [M (⁷⁹Br)⁺+1];405 [M (⁸¹Br)⁺+1], 363, 191.
30. N-(5-bromobenzo[d]isoxazol-3-yl)-2,4-difluorobenzamide,

MS (APCI)=353 [M (⁷⁹Br)⁺+1]; 355 [M (⁸¹Br)⁺+1], 312, 216, 141.
31. N-(5-methylbenzo[d]isoxazol-3-yl)-2-trifluoromethylbenzamide,

MS (APCI)=321 [M⁺+1], 301, 173.
32. 3-fluoro-N-(5-methylbenzo[d]isoxazol-3-yl)benzamide,

MS (APCI)=271 [M⁺+1].
33. N-(4-methoxybenzo[d]isoxazol-3-yl)-3,5-bistrifluoromethyl benzamide,

MS (APCI)=297 [M⁺+1]
34. 3,5-difluoro-N-(4-methoxybenzo[d]isoxazol-3-yl)benzamide,

MS (APCI)=271 [M⁺+1]
35. N-(4-dimethylaminobenzo[d]isoxazol-3-yl)-3,5-bistrifluoromethylbenzamide,

MS (APCI)=418 [M⁺+1].
36. 2-bromo-N-[4-(2,2,2-trifluoroethoxy)benzo[d]isoxazol-3-yl]benzamide,

MS (APCI)=415 [M (⁷⁹Br)⁺+1]; 417 [M (⁸¹Br)⁺+1]
37. 3-chloro-N-[4-(2,2,2-trifluoroethoxy)benzo[d]isoxazol-3-yl]benzamide,

MS (APCI)=371 [M (³⁵Cl)⁺+1]; 373 [M (³⁷Cl)⁺+1], 139.
38. 4-tert-butyl-N-[4-(2,2,2-trifluoroethoxy)benzo[d]isoxazol-3-yl]benzamide,

MS (APCI)=393 [M⁺+1], 161.
39. 2-methoxy-N-[4-(2,2,2-trifluoroethoxy)benzo[d]isoxazol-3-yl]benzamide,

MS (APCI)=367 [M⁺+1], 134.
40. N-(6-chlorobenzo[d]isoxazol-3-yl)-4-iodobenzamide,

MS (APCI)=399 [M (35Cl)⁺+1]; 401 [M (³⁷Cl)⁺+1]
41. naphthalene-2-(6-chlorobenzo[d]isoxazol-3-yl)carboxamide,

MS (APCI)=323 [M⁺+1]
42. N-(6-chlorobenzo[d]isoxazol-3-yl)-3,4-difluorobenzamide,

MS (APCI)=363 [M (³⁵Cl)⁺+1]; 365 [M (³⁷Cl)⁺+1]
43. N-(6-bromobenzo[d]isoxazol-3-yl)-4-fluoro-2-trifluoromethylbenzamide,

MS (APCI)=403 [M (⁷⁹Br)⁺+1]; 405 [M (⁸¹Br)⁺+1]
44. 2,4-dichloro-N-(6-bromobenzo[d]isoxazol-3-yl)-5-fluorobenzamide,

MS (APCI)=403 [M(³⁵Cl³⁵Cl⁷⁹Br)⁺+1]; 405 [M(³⁵Cl³⁷Cl⁷⁹Br)⁺+1], [M(³⁵Cl³⁵Cl⁸¹Br)⁺+1], 407 [M(³⁷Cl³⁷Cl⁷⁹Br)⁺+1], [M(³⁷Cl³⁵Cl⁸¹Br)⁺+1], 409 [M(³⁷Cl³⁷Cl⁸¹Br)⁺+1]
45. N-(6-bromobenzo[d]isoxazol-3-yl)-3-fluoro-4-trifluoromethylbenzamide,

MS (APCI)=403 [M (⁷⁹Br)⁺+1]; 405 [M (³¹Br)⁺+1]
46. N-(6-bromobenzo[d]isoxazol-3-yl)-3-fluoro-5-trifluoromethylbenzamide,

MS (APCI)=403 [M (⁷⁹Br)⁺+1]; 405 [M (⁸¹Br)⁺+1]
47. N-(6-bromobenzo[d]isoxazol-3-yl)-2,3,4-trifluorobenzamide,

MS (APCI)=371 [M (⁷⁹Br)⁺+1]; 373 [M (⁸¹Br)⁺+1]
48. N-(6-bromobenzo[d]isoxazol-3-yl)-4-propylbenzamide,

MS (APCI)=359 [M (⁷⁹Br)⁺+1]; 361 [M (⁸¹Br)⁺+1]
49. N-(6-bromobenzo[d]isoxazol-3-yl)-3,4-difluorobenzamide,

MS (APCI)=353 [M (⁷⁹Br)⁺+1]; 355 [M (⁷⁹Br)⁺+1]
50. furan-2-(5-bromobenzo[d]isoxazol-3-yl)carboxamide,

MS (APCI)=307 [M (⁷⁹Br)⁺+1]; 309 [M (⁷⁹Br)⁺+1]
51. 7-fluoro-N-(4-fluorobenzo[d]isoxazol-3-yl)-2-trifluoromethylbenzamide,

MS (APCI)=343 [M⁺+1]
52. 2,4-dichloro-5-fluoro-N-(7-fluorobenzo[d]isoxazol-3-yl)benzamide,

MS (APCI)=343 [M(³⁵Cl³⁵Cl)⁺+1]; 345 M(³⁵Cl³⁷Cl)⁺+1]; 347 M(³⁷Cl³⁷Cl)⁺+1]
53. 3-fluoro-N-(7-fluorobenzo[d]isoxazol-3-yl)-4-trifluoromethylbenzamide,

MS (APCI)=343 [M⁺+1]
54. 2,3,4-trifluoro-N-(7-fluorobenzo[d]isoxazol-3-yl)benzamide,

MS (APCI)=311 [M⁺+1]
55. N-(7-fluorobenzo[d]isoxazol-3-yl)-4-iodobenzamide,

MS (APCI)=383 [M⁺+1]
56. 2-chloro-N-(7-fluorobenzo[d]isoxazol-3-yl)nicotinamide,

MS (APCI)=292 [M (³⁵Cl)⁺+1]; 294 [M (³⁷Cl)⁺+1]; 256 [M⁺-HCl]
57. naphthalene-2-(7-fluorobenzo[d]isoxazol-3-yl)carboxamide,

MS (APCI)=307 [M⁺+1]
58. N-(74-fluorobenzo[d]isoxazol-3-yl)-4-propylbenzamide,

MS (APCI)=298 [M⁺+1]
59. 3,4-difluoro-N-(7-fluorobenzo[d]isoxazol-3-yl)benzamide,

MS (APCI)=293 [M⁺+1]
60. 2-fluoro-N-(7-fluorobenzo[d]isoxazol-3-yl)-3-trifluoromethylbenzamide,

MS (APCI)=343 [M⁺+1]
61. N-(7-fluorobenzo[d]isoxazol-3-yl)-3-methoxybenzamide,

MS (APCI)=287 [M⁺+1], 135 [M⁺-benzisoxazole]
62. N-(7-fluorobenzo[d]isoxazol-3-yl)-2,2-diphenylacetamide,

MS (APCI)=343 [M⁺+1], 167 [CHPh₂⁺]
63. furan-2-(7-fluorobenzo[d]isoxazol-3-yl)carboxamide,

MS (APCI)=247 [M⁺+1]
64. 2,4-dichloro-N-(4-dimethylaminobenzo[d]isoxazol-3-yl)-5-fluorobenzamide,

MS (APCI)=276 [M(³⁵Cl³⁵Cl)⁺+1]; 278 M(³⁵Cl³⁷Cl)⁺+1]; 280 M(³⁷Cl³⁷Cl)⁺+1], 191 [M(³⁵Cl³⁵Cl)⁺-benzisoxazole]
65. N-(4-dimethylaminobenzo[d]isoxazol-3-yl)-2,3,4-trifluorobenzamide,

MS (APCI)=247 [M⁺+1], 159 [M⁺-benzisoxazole]
66. 2-chloro-N-(4-dimethylaminobenzo[d]isoxazol-3-yl)nicotinamide,

MS (APCI)=317 [M(³⁵Cl³⁵Cl)⁺+1]; 319 [M(³⁵Cl³⁷Cl)⁺+1], 321 [M(³⁷Cl³⁷Cl)⁺+1], 281 [M(³⁵Cl³⁵Cl)⁺-HCl],
67. naphthalene-2-(4-dimethylaminobenzo[d]isoxazol-3-yl)carboxamide,

MS (APCI)=332 [M⁺+1], 155 [M⁺-benzisoxazole]
68. N-(4-dimethylaminobenzo[d]isoxazol-3-yl)-4-propylbenzamide,

MS (APCI)=324 [M⁺+1], 147 [M⁺-benzisoxazole]
69. N-(4-dimethylaminobenzo[d]isoxazol-3-yl)-3,4-difluorobenzamide,

MS (APCI)=324 [M⁺+1], 147 [M⁺-benzisoxazole]
70. N-(4-dimethylaminobenzo[d]isoxazol-3-yl)-2-fluoro-3-trifluoromethylbenzamide,

MS (APCI)=368 [M⁺+1], 191 [M⁺-benzisoxazole]
71. N-(4-dimethylaminobenzo[d]isoxazol-3-yl)-3-methoxybenzamide,

MS (APCI)=312 [M⁺+1], 135 [M⁺-benzisoxazole]
72. N-(4-dimethylaminobenzo[d]isoxazol-3-yl)-2,2-dimethylpropionamide,

MS (APCI)=262 [M⁺+1]
73. cyclohexane-(4-dimethylaminobenzo[d]isoxazol-3-yl)carboxamide,

MS (APCI)=288 [M⁺+1], 178 [benzisoxazole+1]
74. N-(4-dimethylaminobenzo[d]isoxazol-3-yl)-2,2-diphenylacetamide,

MS (APCI)=372 [M⁺+1], 178 [benzisoxazole+1]
75. furan-2-(4-dimethylaminobenzo[d]isoxazol-3-yl)carboxamide,

MS (APCI)=372 [M⁺+1]
76. N-(4-dimethylaminobenzo[d]isoxazol-3-yl)-2,2,3,3,4,4,4-heptafluorobutyramide,

MS (APCI)=372 [M⁺+1]
77. 4-fluoro-N-[4-(2,2,2-trifluoroethoxy)benzo[d]isoxazol-3-yl]-2-trifluoromethyl-benzamide,

MS (APCI)=423 [M⁺+1], 191 [M⁺-benzisoxazole]
78. 2,4-dichloro-5-fluoro-N-[4-(2,2,2-trifluoroethoxy)benzo[d]isoxazol-3-yl]benzamide,

MS (APCI) 423 [M(³⁵Cl³⁵Cl)⁺+1]; 425 [M(³⁵Cl³⁷Cl)⁺+1], 427 [M(³⁷Cl³⁷Cl)⁺+1]
79. 3-fluoro-N-[4-(2,2,2-trifluoroethoxy)benzo[d]isoxazol-3-yl]-4-trifluoromethyl-benzamide,

MS (APCI)=423 [M⁺+1], 191 [M⁺-benzisoxazole]
80. 2,3,4-trifluoro-N-[4-(2,2,2-trifluoroethoxy)benzo[d]isoxazol-3-yl]benzamide,

MS (APCI)=391 [M⁺+1], 159 [M⁺-benzisoxazole]
81. naphthalene-2-[4-(2,2,2-trifluoroethoxy)benzo[d]isoxazol-3-yl]carboxamide,

MS (APCI)=387 [M⁺+1], 155 [M⁺-benzisoxazole]
82. 3,4-difluoro-N-[4-(2,2,2-trifluoroethoxy)benzo[d]isoxazol-3-yl]benzamide,

MS (APCI)=373 [M⁺+1], 141 [M⁺-benzisoxazole]
83. 2-fluoro-N-[4-(2,2,2-trifluoroethoxy)benzo[d]isoxazol-3-yl]-3-trifluoromethyl-benzamide,

MS (APCI)=373 [M⁺+1], 141 [M⁺-benzisoxazole]
85. N-benzo[d]isoxazol-3-yl-2-ethylhexanamide,
86. N-benzo[d]isoxazol-3-yl-2-methylbutyramide,
87. N-benzo[d]isoxazol-3-yl-2,6-difluorobenzamide,
88. N-benzo[d]isoxazol-3-yl-3-fluoro-4-trifluoromethylbenzamide,
89. N-benzo[d]isoxazol-3-yl-2,3,6-trifluorobenzamide,
90. N-benzo[d]isoxazol-3-ylnicotinamide,
91. 5-methylisoxazole-3-benzo[d]isoxazol-3-ylcarboxamide,
92. benzo[b]thiophene-3-benzo[d]isoxazol-3-ylcarboxamide,
93. 2-chloro-N-(7-fluorobenzo[d]isoxazol-3-yl)benzamide,
94. 4-tert-butyl-N-(7-fluorobenzo[d]isoxazol-3-yl)benzamide,
95. N-(7-fluorobenzo[d]isoxazol-3-yl)benzamide,
96. 4-fluoro-N-(7-fluorobenzo[d]isoxazol-3-yl)benzamide,
97. 2-fluoro-N-(7-fluorobenzo[d]isoxazol-3-yl)benzamide,
98. N-(7-fluorobenzo[d]isoxazol-3-yl)-2-methoxybenzamide,
99. N-(7-fluorobenzo[d]isoxazol-3-yl)-2-methylbenzamide,
100. 3,5-difluoro-N-(6-fluorobenzo[d]isoxazol-3-yl)benzamide,
101. naphthalene-1-(6-fluorobenzo[d]isoxazol-3-yl)carboxamide,
102. adamantane-1-(6-fluorobenzo[d]isoxazol-3-yl)carboxamide,
103. 2-bromo-N-(5-fluorobenzo[d]isoxazol-3-yl)benzamide,
104. 4-tert-butyl-N-(5-fluorobenzo[d]isoxazol-3-yl)benzamide,
105. 2,4-difluoro-N-(5-fluorobenzo[d]isoxazol-3-yl)benzamide,
106. naphthalene-2-(5-fluorobenzo[d]isoxazol-3-yl)carboxamide,
107. N-(5-fluorobenzo[d]isoxazol-3-yl)-4-propylbenzamide,
108. 2-chloro-N-(6-chlorobenzo[d]isoxazol-3-yl)benzamide,
109. 4-tert-butyl-N-(6-chlorobenzo[d]isoxazol-3-yl)benzamide,
110. N-(6-chlorobenzo[d]isoxazol-3-yl)-4-fluoro-2-trifluoromethylbenzamide,
111. 2,4-dichloro-N-(6-chlorobenzo[d]isoxazol-3-yl)-5-fluorobenzamide,
112. N-(6-chlorobenzo[d]isoxazol-3-yl)-2-ethylhexanamide,
113. N-(6-chlorobenzo[d]isoxazol-3-yl)-2-methylbutyramide,
114. N-(6-bromobenzo[d]isoxazol-3-yl)-2-chlorobenzamide,
115. N-(6-bromobenzo[d]isoxazol-3-yl)-3,4-dichlorobenzamide,
116. thiophene-2-(6-bromobenzo[d]isoxazol-3-yl)carboxamide,
117. N-(6-bromobenzo[d]isoxazol-3-yl)-3,3-dimethylbutyramide,
118. N-(5-methylbenzo[d]isoxazol-3-yl)benzamide,
119. 4-bromo-N-(5-methylbenzo[d]isoxazol-3-yl)benzamide,
120. 2-chloro-N-(5-methylbenzo[d]isoxazol-3-yl)benzamide,
121. 4-fluoro-N-(5-methylbenzo[d]isoxazol-3-yl)benzamide,
122. 2-fluoro-N-(5-methylbenzo[d]isoxazol-3-yl)benzamide,
123. 3-methyl-N-(5-methylbenzo[d]isoxazol-3-yl)benzamide,
124. 4-tert-butyl-N-(5-methylbenzo[d]isoxazol-3-yl)benzamide,
125. 2-methyl-N-(5-methylbenzo[d]isoxazol-3-yl)benzamide,
126. 2,3-difluoro-N-(5-methylbenzo[d]isoxazol-3-yl)benzamide,
127. 2,4-difluoro-N-(5-methylbenzo[d]isoxazol-3-yl)benzamide,
128. 2-chloro-N-(5-methylbenzo[d]isoxazol-3-yl)nicotinamide,
129. cyclopropane-(5-fluorobenzo[d]isoxazol-3-yl)carboxamide,
130. N-(5-fluorobenzo[d]isoxazol-3-yl)-3,3-dimethyl butyramide,
131. 2,4-difluoro-N-(5-fluorobenzo[d]isoxazol-3-yl)benzamide,
132. 2,5-dimethylfuran-3-(5-fluorobenzo[d]isoxazol-3-yl)carboxamide,
133. N-(5-bromobenzo[d]isoxazol-3-yl)-2,3-difluorobenzamide,
134. 2,5-dimethylfuran-3-(5-bromobenzo[d]isoxazol-3-yl)carboxamide,
135. N-(5-bromobenzo[d]isoxazol-3-yl)-2-methyl butyramide,
136. N-(5-bromobenzo[d]isoxazol-3-yl)-2,6-difluorobenzamide,
137. N-(5-bromobenzo[d]isoxazol-3-yl)-3,4-dimethoxybenzamide,
138. N-(5-bromobenzo[d]isoxazol-3-yl)-4-methylbenzamide,
139. N-(5-bromobenzo[d]isoxazol-3-yl)-2,6-dimethoxybenzamide,
140. 2-bromo-N-(5-bromobenzo[d]isoxazol-3-yl)benzamide,
141. N-(5-bromobenzo[d]isoxazol-3-yl)-5-fluoro-2-trifluoromethylbenzamide,
142. N-(5-bromobenzo[d]isoxazol-3-yl)-3-methoxybenzamide,
143. 3-methyl-N-(5-methyl benzo[d]isoxazol-3-yl)benzamide,
144. 4-cyano-N-(5-methylbenzo[d]isoxazol-3-yl)benzamide,
145. N-(5-methylbenzo[d]isoxazol-3-yl)-2-phenylbutyramide,
146. N-(5-methylbenzo[d]isoxazol-3-yl)-2-methylpentanamide,
147. N-(4-fluorobenzo[d]isoxazol-3-yl)benzamide,
148. 4-chloro-N-(4-fluorobenzo[d]isoxazol-3-yl)benzamide,
149. 2-fluoro-N-(4-fluorobenzo[d]isoxazol-3-yl)benzamide,
150. adamantane-1-(4-fluorobenzo[d]isoxazol-3-yl)carboxamide,
151. adamantane-1-(4-chlorobenzo[d]isoxazol-3-yl)carboxamide,
152. N-(4-chlorobenzo[d]isoxazol-3-yl)benzamide,
153. N-(4-chlorobenzo[d]isoxazol-3-yl)-3-methylbenzamide,
154. N-(4-chlorobenzo[d]isoxazol-3-yl)-2-methylbutyramide,
155. N-(4-chlorobenzo[d]isoxazol-3-yl)-3,3-dimethylbutyramide,
156. N-(4-methoxybenzo[d]isoxazol-3-yl)-2-methylbenzamide,
157. 2-chloro-N-(4-methoxybenzo[d]isoxazol-3-yl)benzamide,
158. N-(4-methoxybenzo[d]isoxazol-3-yl)-2-trifluoromethylbenzamide,
159. N-(4-methoxybenzo[d]isoxazol-3-yl)-2-ethylhexanamide,
160. N-(4-methoxybenzo[d]isoxazol-3-yl)-2-methylbutyramide,
161. N-(4-methoxybenzo[d]isoxazol-3-yl)-2-methylpentanamide,
162. cyclopropane-[4-(2,2,2-trifluoroethoxy)benzo[d]isoxazol-3-yl]carboxamide,
163. 2-methyl-N-[4-(2,2,2-trifluoroethoxy)benzo[d]isoxazol-3-yl]butyramide,
164. 3,3-dimethyl-N-[4-(2,2,2-trifluoroethoxy)benzo[d]isoxazol-3-yl]butyramide,
165. cyclohexane-[4-(2,2,2-trifluoroethoxy)benzo[d]isoxazol-3-yl]carboxamide,
166. 2,5-dimethylfuran-3-[4-(2,2,2-trifluoroethoxy)benzo[d]isoxazol-3-yl]carboxamide,
167. N-[4-(4-methylbenzyloxy)benzo[d]isoxazol-3-yl]-3-nitrobenzamide,
168. N-[4-(4-methyl benzyloxy)benzo[d]isoxazol-3-yl]-4-propylbenzamide,
169. N-(4-dimethylaminobenzo[d]isoxazol-3-yl)-3-fluoro-5-trifluoromethylbenzamide,
170. 3,4-dichloro-N-(4-dimethylaminobenzo[d]isoxazol-3-yl)benzamide,
171. N-(4-dimethylaminobenzo[d]isoxazol-3-yl)-3-nitrobenzamide,
172. adamantane-1-(4-dimethylaminobenzo[d]isoxazol-3-yl)carboxamide,
173. benzo[b]thiophene-2-(4-dimethylaminobenzo[d]isoxazol-3-yl)carboxamide,
174. N-(4-dimethylaminobenzo[d]oxazol-3-yl)-3,3-dimethylbutyramide,
176. N-(6-amino-4,5,7-trifluorobenzo[d]isoxazol-3-yl)-2,6-difluorobenzamide
177. N-(6-amino-4,5,7-trifluorobenzo[d]isoxazol-3-yl)-2,4-difluorobenzamide.

### Pharmacological Data:

The noradrenaline reuptake inhibition (NA uptake inhibition) and the serotonin reuptake inhibition (5-HT uptake inhibition) of the substituted N-benzo[d]isoxazol-3-ylamine derivatives of the invention was determined as described above.

The substituted N-benzo[d]isoxazol-3-ylamine derivatives of the invention exhibit an excellent affinity for the noradrenaline receptor and for the 5-HT receptor.

NA uptake inhibition

| Com- | | | | | | NA uptake |
|---|---|---|---|---|---|---|
| pound | | | | | | inhibition |
| of ex- | | | | | | [%] |
| ample | R¹ | R² | R³ | R⁴ | R⁵ | conc. 10 μM |
| 7 | H | H | H | OCH₃ | CH(Phenyl)₂ | 63 |
| 8 | H | H | Br | H | Cyclopropyl | 62 |
| 9 | H | F | H | H | 3,5-Cl₂-Phenyl | 67 |
| 10 | H | H | H | NH₂ | 4-NO₂-Phenyl | 66 |
| 11 | H | H | H | NH₂ | 1-Naphthyl | 71 |
| 12 | H | H | H | F | 2-Benzo[b]thiophene | 60 |
| 40 | H | Cl | H | H | 4-I-Ph | 61 |
| 41 | H | Cl | H | H | 2-Naphthyl | 61 |
| 42 | H | Cl | H | H | 3,4-F₂Ph | 64 |
| 19 | H | Cl | H | H | 3-CF₃-2-F-Ph | 63 |
| 43 | H | Br | H | H | 2-CF₃-4-FPh | 66 |
| 44 | H | Br | H | H | 2,4-Cl₂-5-FPh | 61 |
| 45 | H | Br | H | H | 4-CF₃-3-F-Ph | 64 |
| 46 | H | Br | H | H | 5-CF₃-3-F-Ph | 65 |
| 47 | H | Br | H | H | 2,3,4-F₃Ph | 66 |
| 48 | H | Br | H | H | 4-n-Pr-Ph | 61 |
| 49 | H | Br | H | H | 3,4-F₂-Ph | 65 |
| 50 | H | H | Br | H | 2-Furyl | 66 |
| 51 | F | H | H | H | 2-CF₃-4-F-Ph | 71 |
| 52 | F | H | H | H | 2,4-Cl₂-5-FPh | 70 |
| 53 | F | H | H | H | 4-CF₃-3-F-Ph | 69 |
| 54 | F | H | H | H | 2,3,4-F₃Ph | 63 |
| 55 | F | H | H | H | 4-I-Ph | 69 |
| 56 | F | H | H | H | 2-Cl-3-Pyridinyl | 72 |
| 57 | F | H | H | H | 2-Naphthyl | 67 |
| 58 | F | H | H | H | 4-n-Pr-Ph | 64 |
| 59 | F | H | H | H | 3,4-F₂-Ph | 67 |
| 60 | F | H | H | H | 3-CF₃-2-F-Ph | 66 |
| 61 | F | H | H | H | 3-OMe-Ph | 66 |
| 62 | F | H | H | H | CHPh₂ | 63 |
| 63 | F | H | H | H | 2-Furyl | 64 |
| 64 | H | H | H | NMe₂ | 2,4-Cl₂-5-F-Ph | 63 |
| 65 | H | H | H | NMe₂ | 2,3,4-F₃-Ph | 68 |
| 66 | H | H | H | NMe₂ | 2-Cl-3-Pyridinyl | 64 |
| 67 | H | H | H | NMe₂ | 2-Naphthyl | 73 |
| 68 | H | H | H | NMe₂ | 4-n-Pr-Ph | 73 |
| 69 | H | H | H | NMe₂ | 3,4-F₂-Ph | 67 |
| 70 | H | H | H | NMe₂ | 3-CF₃-2-F-Ph | 66 |
| 71 | H | H | H | NMe₂ | 3-OMe-Ph | 65 |
| 72 | H | H | H | NMe₂ | C(CH₃)₃ | 66 |
| 73 | H | H | H | NMe₂ | Cyclohexyl | 70 |
| 74 | H | H | H | NMe₂ | CHPh₂ | 70 |
| 75 | H | H | H | NMe₂ | 2-Furyl | 64 |
| 76 | H | H | H | NMe₂ | (CF₂)₂CF₃ | 67 |
| 77 | H | H | H | OCH₂CF₃ | 2-CF₃-4-F-Ph | 70 |
| 78 | H | H | H | OCH₂CF₃ | 2,4-Cl₂-5-FPh | 66 |
| 79 | H | H | H | OCH₂CF₃ | 4-CF₃-3-F-Ph | 62 |
| 80 | H | H | H | OCH₂CF₃ | 2,3,4-F₃Ph | 66 |
| 81 | H | H | H | OCH₂CF₃ | 2-Naphthyl | 65 |
| 82 | H | H | H | OCH₂CF₃ | 3,4-F₂-Ph | 68 |
| 83 | H | H | H | OCH₂CF₃ | 3-CF₃-2-F-Ph | 65 |

5-HT uptake inhibition

| Com- | | | | | | 5 HT uptake |
|---|---|---|---|---|---|---|
| pound | | | | | | inhibition |
| of ex- | | | | | | [%] |
| ample | R¹ | R² | R³ | R⁴ | R⁵ | conc. 10 μM |
| 13 | H | H | H | H | 2-CF₃-Phenyl | 84 |
| 14 | H | H | H | H | 3,4-Cl₂-Phenyl | 69 |
| 15 | H | H | H | H | 2,3-F₂-Phenyl | 86 |
| 16 | H | H | H | N(CH₃)₂ | 3-F-Phenyl | 72 |
| 17 | H | F | H | H | 2-Thiophene | 77 |
| 18 | H | F | H | H | 1-Methylpropyl | 70 |
| 19 | H | Cl | H | H | 2-F-3-CF₃-Phenyl | 77 |
| 20 | H | Cl | H | H | 3-OCH₃-Phenyl | 77 |
| 21 | H | Br | H | H | 4-(tert-Butyl)phenyl | 81 |
| 22 | H | Br | H | H | 2-OCH₃-Phenyl | 79 |
| 23 | H | Br | H | H | 2-CF₃-Phenyl | 86 |
| 24 | H | Br | H | H | Adamantyl | 79 |
| 25 | H | Br | H | H | 1-Methylpropyl | 86 |
| 26 | H | H | F | H | 2-CF₃-Phenyl | 82 |
| 27 | H | H | F | H | 3,4-Cl₂-Phenyl | 89 |
| 28 | H | H | F | H | 2-CH₃-Phenyl | 91 |
| 29 | H | H | Br | H | 2-F-5-CF₃-Phenyl | 83 |
| 30 | H | H | Br | H | 2,4-F₂-Phenyl | 79 |
| 31 | H | H | CH₃ | H | 2-CF₃-Phenyl | 69 |
| 32 | H | H | CH₃ | H | 3-F-Phenyl | 89 |
| 33 | H | H | H | OCH₃ | 3,5-(CF₃)₂-Phenyl | 85 |
| 34 | H | H | H | OCH₃ | 3,5-F₂-Phenyl | 78 |
| 35 | H | H | H | N(CH₃)₂ | 3,5-(CF₃)₂-Phenyl | 86 |
| 36 | H | H | H | OCH₂CF₃ | 2-Br-Phenyl | 84 |
| 37 | H | H | H | OCH₂CF₃ | 3-Cl-Phenyl | 90 |
| 38 | H | H | H | OCH₂CF₃ | 4-(tert-Butyl)phenyl | 77 |
| 39 | H | H | H | OCH₂CF₃ | 2-OCH₃-Phenyl | 82 |

The affinity of the substituted N-benzo[d]isoxazol-3-ylamine derivatives of the invention, of the general formula I, for the mGluR5 receptor was determined as described above.

The substituted N-benzo[d]isoxazol-3-ylamine derivatives of the invention show an excellent affinity for the mGluR5 receptor.

mGluR5 receptor inhibition

| | | | | | | mGluR5 |
|---|---|---|---|---|---|---|
| | | | | | | receptor |
| | | | | | | inhibition |
| | | | | | | of the |
| | | | | | | ³[H]-MPEP |
| | | | | | | binding in |
| Ex. | R¹ | R² | R³ | R⁴ | R⁵ | % (10 μM) |
| 85 | H | H | H | H | 3-Heptyl | 63.19 |
| 86 | H | H | H | H | 2-Butyl | 39.59 |
| 87 | H | H | H | H | 2,6-F₂-Phenyl | 37.01 |
| 88 | H | H | H | H | 3-F-4-CF₃-Phenyl | 30.03 |
| 89 | H | H | H | H | 2,3,6-F₃-Phenyl | 51.10 |
| 90 | H | H | H | H | 3-Pyridyl | 50.28 |
| 91 | H | H | H | H | 3-(5-Methyl)- | 35.00 |
| | | | | | isoxazolyl | |
| 92 | H | H | H | H | 2-Benzo[b]- | 55.98 |
| | | | | | thiophenyl | |
| 93 | F | H | H | H | 2-Cl-Phenyl | 35.39 |
| 94 | F | H | H | H | 4-(tert-Butyl)- | 30.79 |
| | | | | | phenyl | |
| 95 | F | H | H | H | Phenyl | 45.82 |
| 96 | F | H | H | H | 4-F-Phenyl | 30.47 |
| 98 | F | H | H | H | 2-OCH₃-Phenyl | 32.01 |
| 99 | F | H | H | H | 2-CH₃-Phenyl | 43.05 |
| 100 | H | F | H | H | 3,5-F₂-Phenyl | 33.33 |
| 101 | H | F | H | H | 1-Naphthyl | 48.99 |
| 102 | H | F | H | H | Adamantyl | 52.11 |
| 103 | H | H | F | H | 2-Br-Phenyl | 41.17 |
| 104 | H | H | F | H | 4-(tert-Butyl)- | 44.61 |
| | | | | | phenyl | |
| 28 | H | H | F | H | 2-CH₃-Phenyl | 35.00 |
| 105 | H | H | F | H | 2,4-F₂-Phenyl | 53.77 |
| 106 | H | H | F | H | 2-Naphthyl | 33.72 |
| 107 | H | H | F | H | 4-Propyl-phenyl | 42.12 |
| 108 | H | Cl | H | H | 2-Cl-Phenyl | 34.73 |
| 109 | H | Cl | H | H | 4-(tert-Butyl)- | 35.20 |
| | | | | | phenyl | |
| 110 | H | Cl | H | H | 4-F-2-CF₃-Phenyl | 35.96 |
| 111 | H | Cl | H | H | 2,4-Cl₂-5-F- | 43.05 |
| | | | | | Phenyl | |
| 112 | H | Cl | H | H | 3-Heptyl | 35.12 |
| 113 | H | Cl | H | H | 2-Butyl | 49.16 |
| 49 | H | Br | H | H | 3,4-F₂-Phenyl | 36.90 |
| 114 | H | Br | H | H | 2-Cl-Phenyl | 37.51 |
| 115 | H | Br | H | H | 3,4-Cl₂-Phenyl | 35.84 |
| 116 | H | Br | H | H | 2-Thiophenyl | 37.71 |
| 117 | H | Br | H | H | 2,2-Dimethyl- | 30.78 |
| | | | | | propyl | |
| 118 | H | H | CH₃ | H | Phenyl | 69.01 |
| 119 | H | H | CH₃ | H | 4-Br-Phenyl | 37.90 |
| 120 | H | H | CH₃ | H | 2-Cl-Phenyl | 47.86 |
| 121 | H | H | CH₃ | H | 4-F-Phenyl | 32.22 |
| 122 | H | H | CH₃ | H | 2-F-Phenyl | 41.03 |
| 123 | H | H | CH₃ | H | 3-CH₃-Phenyl | 89.70 |
| 124 | H | H | CH₃ | H | 4-(tert-Butyl)- | 43.51 |
| | | | | | phenyl | |
| 125 | H | H | CH₃ | H | 2-CH₃-Phenyl | 40.62 |
| 126 | H | H | CH₃ | H | 2,3-F₂-Phenyl | 39.49 |
| 127 | H | H | CH₃ | H | 2,4-F₂-Phenyl | 43.22 |
| 128 | H | H | CH₃ | H | 3-(2-Cl)-Pyridyl | 33.12 |
| 129 | H | H | F | H | Cyclopropyl | 38.82 |
| 130 | H | H | F | H | 2,2-Dimethylpropyl | 31.64 |
| 131 | H | H | F | H | 2,4-F₂-Phenyl | 42.52 |
| 132 | H | H | F | H | 3-(2,5-Dimethyl)- | 36.88 |
| | | | | | furanyl | |
| 30 | H | H | Br | H | 2,4-F₂-Phenyl | 45.22 |
| 133 | H | H | Br | H | 2,3-F₂-Phenyl | 34.56 |
| 134 | H | H | Br | H | 3-(2,5-Dimethyl)- | 43.11 |
| | | | | | furanyl | |
| 135 | H | H | Br | H | 2-Butyl | 40.00 |
| 136 | H | H | Br | H | 2,6-F₂-Phenyl | 40.76 |
| 137 | H | H | Br | H | 3,4-(OCH₃)₂- | 31.25 |
| | | | | | Phenyl | |
| 138 | H | H | Br | H | 4-CH₃-Phenyl | 31.96 |
| 139 | H | H | Br | H | 2,6-(OCH₃)₂- | 41.55 |
| | | | | | Phenyl | |
| 140 | H | H | Br | H | 2-Br-Phenyl | 38.42 |
| 141 | H | H | Br | H | 5-F-2-CF₃- | 33.08 |
| | | | | | Phenyl | |
| 142 | H | H | Br | H | 3-OCH₃-Phenyl | 37.84 |
| 32 | H | H | CH₃ | H | 3-F-Phenyl | 40.06 |
| 143 | H | H | CH₃ | H | 3-CH₃-Phenyl | 44.66 |
| 144 | H | H | CH₃ | H | 4-CN-Phenyl | 48.79 |
| 145 | H | H | CH₃ | H | 1-Phenyl-propyl | 43.51 |
| 146 | H | H | CH₃ | H | 2-Pentyl | 33.97 |
| 147 | H | H | H | F | Phenyl | 31.35 |
| 148 | H | H | H | F | 4-Cl-Phenyl | 79.45 |
| 149 | H | H | H | F | 2-F-Phenyl | 32.45 |
| 150 | H | H | H | F | Adamantyl | 53.18 |
| 151 | H | H | H | Cl | Adamantyl | 74.05 |
| 152 | H | H | H | Cl | Phenyl | 31.62 |
| 153 | H | H | H | Cl | 3-CH₃-Phenyl | 63.76 |
| 154 | H | H | H | Cl | 2-Butyl | 30.02 |
| 155 | H | H | H | Cl | 2,2-Dimethyl- | 52.37 |
| | | | | | propyl | |
| 156 | H | H | H | OCH₃ | 2-CH₃-Phenyl | 33.76 |
| 157 | H | H | H | OCH₃ | 2-Cl-Phenyl | 32.39 |
| 158 | H | H | H | OCH₃ | 2-CF₃-Phenyl | 36.13 |
| 159 | H | H | H | OCH₃ | 3-Heptyl | 43.24 |
| 160 | H | H | H | OCH₃ | 2-Butyl | 53.97 |
| 161 | H | H | H | OCH₃ | 2-Pentyl | 38.49 |
| 39 | H | H | H | OCH₂CF₃ | 2-OCH₃-Phenyl | 30.50 |
| 36 | H | H | H | OCH₂CF₃ | 2-Br-Phenyl | 30.31 |
| 77 | H | H | H | OCH₂CF₃ | 4-F-2-CF₃-Phenyl | 37.98 |
| 82 | H | H | H | OCH₂CF₃ | 3,4-F₂-Phenyl | 41.99 |
| 162 | H | H | H | OCH₂CF₃ | Cyclopropyl | 30.99 |
| 163 | H | H | H | OCH₂CF₃ | 2-Butyl | 35.96 |
| 164 | H | H | H | OCH₂CF₃ | 2,2-Dimethyl- | 33.37 |
| | | | | | propyl | |
| 165 | H | H | H | OCH₂CF₃ | Cyclohexyl | 38.04 |
| 166 | H | H | H | OCH₂CF₃ | 3-(2,5-Dimethyl)- | 32.39 |
| | | | | | furanyl | |
| 167 | H | H | H | OCH₂- | 3-NO₂-Phenyl | 39.74 |
| | | | | (4-CH₃- | | |
| | | | | Phenyl) | | |
| 168 | H | H | H | OCH₂- | 4-Propyl-phenyl | 32.20 |
| | | | | (4-CH₃- | | |
| | | | | Phenyl) | | |
| 169 | H | H | H | N(CH₃)₂ | 3-F-5-CF₃-Phenyl | 36.77 |
| 170 | H | H | H | N(CH₃)₂ | 3,4-Cl₂-Phenyl | 37.97 |
| 171 | H | H | H | N(CH₃)₂ | 3-NO₂-Phenyl | 42.31 |
| 73 | H | H | H | N(CH₃)₂ | Cyclohexyl | 30.05 |
| 172 | H | H | H | N(CH₃)₂ | Adamantyl | 51.40 |
| 173 | H | H | H | N(CH₃)₂ | 2-Benzo[b]- | 43.55 |
| | | | | | thiophenyl | |
| 174 | H | H | H | N(CH₃)₂ | 2,2-Dimethyl- | 30.66 |
| | | | | | propyl | |
| 176 | F | NH₂ | F | F | 2,6-F₂-Phenyl | 36.07 |
| 177 | F | NH₂ | F | F | 2,4-F₂-Phenyl | 30.80 |

1-34. (canceled)

35. A pharmaceutical composition comprising at least one pharmaceutically acceptable carrier or excipient and a substituted N-benzo[d]isoxazol-3-yl amine compound corresponding to formula I 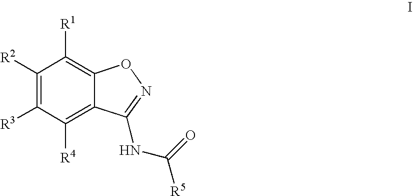
wherein
R¹, R², R³ and R⁴ are each independently H, F, Cl, Br, I, —CN, —NC, —NO₂, —SO₃H, —S(═O₂)NH₂, —NH₂, —OH, —SH, linear or branched C₁₋₁₀ alkyl, —OR⁶, —O—(CH₂)—R⁷, —SR⁸, —S—(CH₂)—R⁹, —NR¹⁰R¹¹, —NR¹³—C(═O)—R¹², —C(═O)—NH₂, —C(═O)—R¹⁴, —C(═O)—OH or —C(═O)—OR¹⁵;
R⁵ is a linear or branched, optionally substituted C₁₋₁₀ alkyl group;
a linear or branched, optionally substituted C₂₋₁₀ alkenyl group;
an unsaturated or saturated, optionally substituted 3-, 4-, 5-, 6-, 7-, 8- or 9-membered cycloaliphatic group which optionally may be bridged by 1 or 2 linear or branched C₁₋₅-alkylene groups;
an imidazolidinonyl group which optionally may be substituted by 1 or 2 substituents independently selected from the group consisting of methyl, ethyl, n-propyl, isopropyl, n-butyl, isobutyl and tert-butyl;
an optionally substituted 6- to 10-membered aryl group which optionally may be fused to a saturated or unsaturated, optionally substituted mono- or polycyclic ring system;
a group selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl, 4-methyl-3-nitrophenyl and 5-nitrofuranyl;
an optionally substituted 5- to 14-membered heteroaryl group; —(CH₂)ₙ—C(═O)—OR¹⁶ wherein n is 0, 1, 2, 3, 4 or 5;
—CR¹⁷R¹⁸—O—C(═O)—R¹⁹;
—(CHR²⁰)—(CH₂)ₚ—R²¹ with p=0 or 1; or
—(CH═CH)—R²²;
R⁶, R⁸, R¹⁴ and R¹⁵ are each independently a linear or branched, optionally substituted C₁₋₁₀ alkyl group;
a linear or branched, optionally substituted C₂₋₁₀ alkenyl group; or
an optionally substituted 5- to 14-membered aryl or heteroaryl group;
R⁷ and R⁹ are each independently an optionally substituted 5- to 14-membered aryl or heteroaryl group;
R¹⁰ and R¹¹ are each independently hydrogen or are a linear or branched C₁₋₁₀ alkyl group;
R¹², R¹³, R¹⁶ and R¹⁹ are each independently a linear or branched C₁₋₁₀ alkyl group;
R¹⁷ is a linear or branched C₁₋₁₀ alkyl group or an optionally substituted 6- to 10-membered aryl group;
R¹⁸ and R²⁰ are each independently hydrogen, are a linear or branched C₁₋₁₀ alkyl group, or an optionally substituted 6- to 10-membered aryl group;
R²¹ is an unsaturated or saturated, optionally substituted 3-, 4-, 5-, 6-, 7-, 8- or 9-membered cycloaliphatic group, an optionally substituted 6- to 10-membered aryl group, or an optionally substituted heteroaryl group selected from the group consisting of thiophenyl, furanyl, benzo[b]furanyl and benzo[b]thiophenyl; and
R²² is an optionally substituted 6- to 10-membered aryl group;
wherein
the aforementioned, optionally substituted C₁₋₁₀ alkyl groups may be unsubstituted or substituted by 1, 2, 3, 4, 5, 6, 7, 8 or 9 substituents independently selected from the group consisting of F, Cl, Br, I, —OH, —SH, —CN and —NO₂;
the aforementioned, optionally substituted C₂₋₁₀ alkenyl groups may be unsubstituted or substituted by 1, 2, 3, 4, 5, 6, 7, 8 or 9 substituents independently selected from the group consisting of F, Cl, Br, I, —OH, —SH, —CN and —NO₂;
the aforementioned, optionally substituted cycloaliphatic groups may be unsubstituted or substituted by 1, 2, 3, 4 or 5 substituents independently selected from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—H, —C(═O)—C₁₋₅-alkyl, —C(═O)—O—C₁₋₅-alkyl, —(CH₂)—C(═O)—O—C₁₋₅-alkyl, —NH—Cl s-alkyl, —N(C₁₋₅-alkyl)₂, —(CH₂)-benzo[b]furanyl, —O-phenyl, —O-benzyl, phenyl, benzyl, naphthyl and —(CH₂)-naphthyl;
wherein the cyclic part of the groups —O-phenyl, —O-benzyl, phenyl, —(CH₂)-benzo[b]furanyl, benzyl, naphthyl and —(CH₂)-naphthyl optionally may be substituted by 1, 2, 3, 4 or 5 substituents independently selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl;
the aforementioned optionally substituted aryl or heteroaryl groups may be unsubstituted or substituted by 1, 2, 3, 4 or 5 substituents independently selected from the group consisting of F, Cl, Br, I, —CN, —CF₃, —SF₅, —OH, —O—C₁₋₅-alkyl, —O—C₂₋₅-alkenyl, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl, pyridinyl and benzyl;
wherein the cyclic part of the groups —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl optionally may be substituted by 1, 2, 3, 4 or 5 substituents independently selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl,
and
the aforementioned heteroaryl groups each may optionally contain 1, 2, 3, 4 or 5 heteroatom(s) independently selected from the group consisting of oxygen, nitrogen and sulfur as ring member(s);
the rings of the aforementioned optionally substituted mono- or polycyclic ring systems may be unsubstituted or substituted by 1, 2, 3, 4 or 5 substituents independently selected from the group consisting of oxo (═O), thioxo (═S), F, Cl, Br, I, —CN, —CF₃, —SF₅, —OH, —O—C₁₋₅-alkyl, —O—C₂₋₅-alkenyl, —NH₂, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl and —S(═O)₂—C₁₋₅-alkyl; and
the rings of the aforementioned mono- or polycyclic ring systems each have 5, 6 or 7 members and may each contain 1, 2 or 3 heteroatom(s) as ring member(s), which are independently selected from the group consisting of oxygen, nitrogen and sulfur;
or a salt or solvate thereof.

36. A composition according to claim 35, wherein said compound is in the form of an isolated or purified stereoisomer.

37. A composition according to claim 35, wherein said compound is in the form of a racemic mixture of stereoisomers.

38. A composition according to claim 35, wherein
R⁶, R⁸, R¹⁴ and R¹⁵ are each independently
a group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃, —CH₂—CH₂—CH₂—CN, 1-propenyl, 2-propenyl, 1-butenyl, 2-butenyl, 3-butenyl and 2-methyl-1-propenyl; or
a group selected from the group consisting of phenyl, naphthyl, thiophenyl, furanyl and pyridinyl, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

39. A composition according to claim 35, wherein R⁷ and R⁹ each independently denote a group selected from the group consisting of phenyl, naphthyl, thiophenyl, furanyl and pyridinyl, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

40. A composition according to claim 35, wherein R¹⁰ and R¹¹ each independently denote hydrogen or an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

41. A composition according to claim 35, wherein R¹², R¹³, R¹⁶ and R¹⁹ each independently denote an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

42. A composition according to claim 35, wherein R¹⁷ is
an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl, or
a phenyl or naphthyl group, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

43. A composition according to claim 35, wherein
R¹⁸ and R²⁰ each independently denote
hydrogen; or
an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl; or
a phenyl, or naphthyl group, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

44. A composition according to claim 35, wherein R²¹ is
a cycloaliphatic group selected from the group consisting of cyclopropyl, cyclobutyl, cyclopentyl, cyclohexyl, cycloheptyl, cyclopentenyl, cyclohexenyl and cycloheptenyl, wherein said cycloaliphatic group optionally may be substituted by 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂ and —N(CH(CH₃)₂)₂; or
a phenyl group optionally substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

45. A composition according to claim 35, wherein R²² is a phenyl group optionally substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

46. A composition according to claim 35, wherein
R¹, R², R³ and R⁴ each independently are H, F, Cl, Br, I, —CN, —NC, —NO₂, —SO₃H, —S(═O₂)NH₂, —NH₂, —OH, —SH, methyl, ethyl, n-propyl, isopropyl, n-butyl, sec-butyl, isobutyl, tert-butyl, n-pentyl, sec-pentyl, n-hexyl, —OR⁶, —O—(CH₂)—R⁷, —SR⁸, —S—(CH₂)—R⁹, —NR¹⁰R¹¹, —NR¹³—C(═O)—R¹², —C(═O)—NH₂, —C(═O)—R¹⁴, —C(═O)—OH or —C(═O)—OR¹⁵;
R⁵ is
a group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃, —CH₂—CH₂—CH₂—CN, 1-propenyl, 2-propenyl, 1-butenyl, 2-butenyl, 3-butenyl and 2-methyl-1-propenyl;
a cycloaliphatic group selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantyl, wherein said cycloaliphatic group optionally may be substituted by 1, 2, 3, 4 or 5 substituents independently selected from the group consisting of oxo (═O), thioxo (═S), F, Cl, Br, I, —CN, —CF₃, —SF₅, —O—CH₃, —O—C₂H₅, —O—C₃H₇, methyl, ethyl, n-propyl, isopropyl, n-butyl, sec-butyl, isobutyl, tert-butyl, phenyl and benzyl; wherein the cyclic part of the phenyl and benzyl groups optionally may be substituted by 1, 2, 3, 4 or 5 substituents independently selected from the group consisting of F, Cl, Br, methyl, ethyl and n-propyl;
an imidazolidinonyl group;
a phenyl or naphthyl group, wherein said group optionally may be substituted by 1, 2, 3, 4 or 5 substituents independently selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, phenyl and benzyl; wherein the cyclic part of the phenyl and benzyl groups optionally may be substituted by 1, 2, 3, 4 or 5 substituents independently selected from the group consisting of F, Cl, Br, —OH, —CF₃, —SF₅, —CN, —NO₂, —O—CH₃, —O—C₂H₅, —O—C₃H₇ methyl, ethyl, —O—CF₃ and —S—CF₃;
a group selected from the group consisting of [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl, wherein said group optionally may be substituted by 1, 2, 3, 4 or 5 substituents independently selected from the group consisting of methyl, ethyl, n-propyl, isopropyl, n-butyl, sec-butyl, isobutyl and tert-butyl;
a group selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl, 4-methyl-3-nitrophenyl and 5-nitrofuranyl;
a heteroaryl group selected from the group consisting of thiophenyl, furanyl, pyrazolyl, pyrazinyl, pyranyl, triazolyl, pyridinyl, indolyl, benzo[b]furanyl, benzo[b]thiophenyl, thiazolyl, oxazolyl, isoxazolyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl, wherein said heteroaryl group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CH₂—CH═CH₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, dihydrobenzo[b]furanyl, —O-phenyl, —O-benzyl, —S-phenyl, —S-benzyl, phenyl, pyridinyl and benzyl; wherein the cyclic part of the groups —S(═O)₂-phenyl, —O-phenyl, —O-benzyl, —S-phenyl, —S-benzyl, phenyl, dihydro[b]benzofuranyl, pyridinyl and benzyl optionally may be substituted by 1, 2, 3, 4 or 5 substituents independently selected from the group consisting of F, Cl, Br, —CF₃, —CN, —NO₂, —O—CH₃, —O—C₂H₅, —O—C₃H₇, methyl, ethyl, n-propyl, isopropyl, n-butyl, sec-butyl, isobutyl, tert-butyl, —O—CF₃ and —S—CF₃;
—(CH₂)ₙ—C(═O)—OR¹⁶, wherein n is 0, 1, 2, 3 or 4;
—CR¹⁷R¹⁸—O—C(═O)—R¹⁹;
—(CHR²⁰)—(CH₂)ₚ—R²¹, wherein p is 0 or 1; or
—(CH═CH)—R²²;
R⁶, R⁸, R¹⁴ and R¹⁵ each independently denote a group selected from the group consisting of methyl, ethyl, n-propyl, isopropyl, n-butyl, isobutyl, tert-butyl, —CF₃, —CFH₂, —CF₂H, —CF₂—CF₃, —CH₂—CF₃ and —CF₂—CF₂—CF₃;
R⁷ and R⁹ each independently denote a phenyl group optionally substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
R¹⁰ and R¹¹ each independently denote an alkyl group selected from the group consisting of methyl, ethyl, n-propyl, isopropyl, n-butyl, isobutyl and tert-butyl;
R¹², R¹³, R¹⁶ and R¹⁹ each independently denote an alkyl group selected from the group consisting of methyl, ethyl, n-propyl, isopropyl and tert-butyl;
R¹⁷ is an alkyl group selected from the group consisting of methyl, ethyl, n-propyl, isopropyl and n-butyl; or a phenyl group;
R¹⁸ is hydrogen, or an alkyl group selected from the group consisting of methyl, ethyl, n-propyl, isopropyl, n-butyl, isobutyl and tert-butyl;
R²⁰ is hydrogen; or an alkyl group selected from the group consisting of methyl, ethyl, n-propyl, isopropyl, n-butyl, isobutyl and tert-butyl; or a phenyl group;
R²¹ is
a cycloaliphatic group selected from the group consisting of cyclopropyl, cyclobutyl, cyclopentyl, cyclohexyl, cycloheptyl, cyclopentenyl, cyclohexenyl and cycloheptenyl;
a phenyl group optionally substituted by 1, 2, 3, 4 or 5 substituents independently selected from the group consisting of F, Cl, Br, I, —CN, —CF₃, —SF₅, —O—CH₃, —O—C₂H₅, —O—C₃H₇, —O—CF₃, —O—CHF₂, —O—CH₂F, methyl, ethyl, n-propyl, isopropyl, n-butyl, sec-butyl, isobutyl and tert-butyl; or
a thiophenyl group; and
R²² is a phenyl group optionally substituted by 1, 2, 3, 4 or 5 substituents independently selected from the group consisting of F, Cl, Br, and —CF₃.

47. A composition according to claim 35, wherein
R¹ is H, F, Cl or Br;
R² is H, F, Cl, Br or —NH₂;
R³ is H, F, Cl, Br, methyl, ethyl, n-propyl, isopropyl, n-butyl, isobutyl or tert-butyl;
R⁴ is H, F, Cl, Br, —NH₂, methyl, ethyl, n-propyl, isopropyl, n-butyl, isobutyl, tert-butyl, —OR⁶, —O—(CH₂)—R⁷ or —NR¹⁰R¹¹;
R⁵ is
a group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CF₂—CF₃, —CH₂—CF₃, —CF₂—CF₂—CF₃, —CH₂—CH₂—CF₃ and —CF₂—CF₂—CF₂—CF₃;
a cycloaliphatic group selected from the group consisting of cyclopropyl, cyclobutyl, cyclopentyl, cyclohexyl, cycloheptyl, cyclopentenyl, cyclohexenyl, cycloheptenyl, (6,6)-dimethyl-[3.1.1]-bicycloheptyl and adamantyl;
a group selected from the group consisting of phenyl, 2-trifluoromethylphenyl, 2-nitrophenyl, 2-dimethylaminophenyl, 2-diethylaminophenyl, 2-amino-phenyl, 2-ethylphenyl, 2-methylbenzoate, 2-bromophenyl, 2-chlorophenyl, 2-fluorophenyl, 2-methylphenyl, 2-methoxyphenyl, 2-ethoxyphenyl, 2-cyanophenyl, 2-acetylphenyl, 2-dimethylaminosulfonylphenyl, 3-chlorophenyl, 3-methylphenyl, 3-nitrophenyl, 3-trifluoromethylphenyl, 3-fluorophenyl, 3-bromophenyl, 3-dimethylaminophenyl, 3-diethylaminophenyl, 3-aminophenyl, 3-methoxyphenyl, 3-ethylphenyl, 3-ethoxyphenyl, 3-cyanophenyl, 3-acetylphenyl, 3-phenylphenyl, 3-dimethylaminosulfonylphenyl, 4-bromophenyl, 4-methoxyphenyl, 4-chlorophenyl, 4-fluorophenyl, 4-tert-butylphenyl, 4-cyanophenyl, 4-nitrophenyl, 4-methylphenyl, 4-phenylphenyl, 4-trifluoromethylphenyl, 4-dimethylaminophenyl, 4-diethylaminophenyl, 4-aminophenyl, 4-iodophenyl, 4-n-propylphenyl, 4-di-n-propylaminosulfonylphenyl, 4-diethyl-aminosulfonylphenyl, 4-dimethylaminosulfonylphenyl, 4-ethylphenyl, 4-ethoxyphenyl, 4-methylbenzoate, 4-acetylphenyl, 2-fluoro-3-trifluoromethylphenyl, (2,3)-difluorophenyl, (2,3)-dimethylphenyl, (2,3)-dichlorophenyl, 3-fluoro-2-trifluoromethylphenyl, (2,4)-dichlorophenyl, (2,4)-difluorophenyl, 4-fluoro-2-trifluoromethylphenyl, (2,4)-dimethoxyphenyl, 2-chloro-4-fluorophenyl, (2,4)-dibromophenyl, 2-fluoro-4-trifluoromethylphenyl, (2,5)-difluorophenyl, 2-fluoro-5-trifluoromethylphenyl, 5-fluoro-2-trifluoromethylphenyl, 5-chloro-2-trifluoromethylphenyl, 5-bromo-2-trifluoromethylphenyl, (2,5)-dimethoxyphenyl, (2,5)-bistrifluoromethylphenyl, (2,5)-dichlorophenyl, (2,5)-dibromophenyl, 2-fluoro-6-trifluoromethylphenyl, (2,6)-dimethoxyphenyl, (2,6)-dimethylphenyl, 2-chloro-6-fluorophenyl, 2-bromo-6-chlorophenyl, 2-bromo-6-fluorophenyl, (2,6)-difluorophenyl, (2,6)-dibromophenyl, (2,6)-dichlorophenyl, (3,4)-dichlorophenyl, 4-chloro-3-nitrophenyl, (3,4)-dimethoxyphenyl, 4-fluoro-3-trifluoromethylphenyl, 3-fluoro-4-trifluoromethylphenyl, (3,4)-difluorophenyl, 4-bromo-3-methylphenyl, 4-bromo-5-methylphenyl, 3-chloro-4-fluorophenyl, 4-fluoro-3-nitrophenyl, 4-bromo-3-nitrophenyl, (3,4)-dibromophenyl, 4-chloro-3-methylphenyl, 4-fluoro-3-methylphenyl, 4-methyl-3-nitrophenyl, (3,5)-dimethoxyphenyl, (3,5)-bis-trifluoromethylphenyl, (3,5)-difluorophenyl, (3,5)-dinitrophenyl, (3,5)-dichlorophenyl, 3-fluoro-5-trifluoromethylphenyl, 5-fluoro-3-trifluoromethylphenyl, (3,5)-dibromophenyl, 5-chloro-4-fluorophenyl, 5-chloro-4-fluorophenyl, 5-bromo-4-methylphenyl, (2,3,4)-trifluorophenyl, (2,3,4)-trichlorophenyl, (2,3,6)-trifluorophenyl, (2,4,6)-trichlorophenyl, (2,4,5)-trifluorophenyl, (2,4,5)-trichlorophenyl, (2,4)-dichloro-5-fluorophenyl, (2,4,6)-trichlorophenyl, (2,4,6)-trimethylphenyl, (2,4,6)-trifluorophenyl, (2,4,6)-trimethoxyphenyl, (3,4,5)-trimethoxyphenyl, (2,3,4,5)-tetrafluorophenyl and (2,3,4,5,6)-pentafluorophenyl;
a naphthyl group;
a heteroaryl group selected from the group consisting of thiophenyl, furanyl, pyridinyl, benzo[b]furanyl, benzo[b]thiophenyl, oxazolyl and isoxazolyl, wherein said heteroaryl group optionally may be substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
—(CH₂)ₙ—C(═O)—OR¹⁶, wherein n is 1 or 2; or
—(CHR²⁰)—(CH₂)ₚ—R²¹ with p=0 or 1;
R⁶ is selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R⁷ is a phenyl group optionally substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
R¹⁰ and R¹¹ each independently denote an alkyl group selected from the group consisting of methyl, ethyl, n-propyl, isopropyl, n-butyl, isobutyl and tert-butyl;
R¹⁶ is an alkyl group selected from the group consisting of methyl, ethyl, n-propyl, isopropyl, n-butyl, isobutyl and tert-butyl;
R²⁰ is hydrogen; an alkyl group selected from the group consisting of methyl, ethyl, n-propyl, isopropyl, n-butyl, isobutyl and tert-butyl; or a phenyl group, and
R²¹ is
a cycloaliphatic group selected from the group consisting of cyclopropyl, cyclobutyl, cyclopentyl, cyclohexyl, cycloheptyl, cyclopentenyl, cyclohexenyl and cycloheptenyl;
a phenyl group which may be substituted by 1, 2, 3, 4 or 5 substituents independently selected from the group consisting of F, Cl, Br, I, —CN, —CF₃, —SF₅, —O—CH₃, —O—C₂H₅, —O—C₃H₇, —O—CF₃, —O—CHF₂, —O—CH₂F, methyl, ethyl, n-propyl, isopropyl, n-butyl, sec-butyl, isobutyl and tert-butyl; or
a thiophenyl group.

48. A composition according to claim 35, wherein said compound is selected from the group consisting of:
[1] thiophene-2-benzo[d]isoxazol-3-ylcarboxamide,
[2] N-benzo[d]isoxazol-3-ylsuccinic acid methyl ester
[3] naphthalene-2-benzo[d]isoxazol-3-ylcarboxamide,
[4] adamantane-2-benzo[d]isoxazol-3-ylcarboxamide,
[5] cyclohexane-benzo[d]isoxazol-3-ylcarboxamide,
[6] N-benzo[d]isoxazol-3-yl-2,2-dimethylpropionamide,
[7] N-(4-methoxybenzo[d]isoxazol-3-yl)-2,2-diphenylacetamide,
[8] cyclopropane(5-bromobenzo[d]isoxazol-3-yl)carboxamide,
[9] 3,5-dichloro-N-(6-fluorobenzo[d]isoxazol-3-yl)benzamide,
[10] N-(4-aminobenzo[d]isoxazol-3-yl)-4-nitrobenzamide,
[11] naphthalene-1-(4-aminobenzo[d]isoxazol-3-yl)carboxamide,
[12] benzo[b]thiophene-2-(4-fluorobenzo[d]isoxazol-3-yl)carboxamide,
[13] N-benzo[d]isoxazol-3-yl-2-trifluoromethylbenzamide,
[14] N-benzo[d]isoxazol-3-yl-3,4-dichlorobenzamide
[15] N-benzo[d]isoxazol-3-yl-2,3-difluorobenzamide,
[16] N-(4-dimethylaminobenzo[d]isoxazol-3-yl)-3-fluorobenzamide,
[17] thiophene-2-(6-fluorobenzo[d]isoxazol-3-yl)carboxamide,
[18] N-(6-fluorobenzo[d]isoxazol-3-yl)-2-methylbutyramide,
[19] N-(6-chlorobenzo[d]isoxazol-3-yl)-2-fluoro-3-trifluoromethylbenzamide,
[20] N-(6-chlorobenzo[d]isoxazol-3-yl)-3-methoxybenzamide,
[21] N-(6-bromobenzo[d]isoxazol-3-yl)-4-tert-butylbenzamide,
[22] N-(6-bromobenzo[d]isoxazol-3-yl)-2-methoxybenzamide,
[23] N-(6-bromobenzo[d]isoxazol-3-yl)-2-trifluoromethylbenzamide,
[24] adamantane-2-(6-bromobenzo[d]isoxazol-3-yl)carboxamide,
[25] N-(6-bromobenzo[d]isoxazol-3-yl)-2-methylbutyramide,
[26] N-(5-fluorobenzo[d]isoxazol-3-yl)-2-trifluoromethylbenzamide,
[27] 3,4-dichloro-N-(5-fluorobenzo[d]isoxazol-3-yl)benzamide,
[28] N-(5-fluorobenzo[d]isoxazol-3-yl)-2-methylbenzamide,
[29] N-(5-bromobenzo[d]isoxazol-3-yl)-2-fluoro-5-trifluoromethylbenzamide,
[30] N-(5-bromobenzo[d]isoxazol-3-yl)-2,4-difluorobenzamide,
[31] N-(5-methylbenzo[d]isoxazol-3-yl)-2-trifluoromethylbenzamide,
[32] 3-fluoro-N-(5-methylbenzo[d]isoxazol-3-yl)benzamide,
[33] N-(4-methoxybenzo[d]isoxazol-3-yl)-3,5-bis-trifluoromethylbenzamide,
[34] 3,5-difluoro-N-(4-methoxybenzo[d]isoxazol-3-yl)benzamide,
[35] N-(4-dimethylaminobenzo[d]isoxazol-3-yl)-3,5-bis-trifluoromethylbenzamide,
[36] 2-bromo-N-[4-(2,2,2-trifluoroethoxy)benzo[d]isoxazol-3-yl]benzamide,
[37] 3-chloro-N-[4-(2,2,2-trifluoroethoxy)benzo[d]isoxazol-3-yl]benzamide,
[38] 4-tert-butyl-N-[4-(2,2,2-trifluoroethoxy)benzo[d]isoxazol-3-yl]benzamide,
[39] 2-methoxy-N-[4-(2,2,2-trifluoroethoxy)benzo[d]isoxazol-3-yl]benzamide,
[40] N-(6-chlorobenzo[d]isoxazol-3-yl)-4-iodobenzamide,
[41] naphthalene-2-(6-chlorobenzo[d]isoxazol-3-yl)carboxamide,
[42] N-(6-chlorobenzo[d]isoxazol-3-yl)-3,4-difluorobenzamide,
[43] N-(6-bromobenzo[d]isoxazol-3-yl)-4-fluoro-2-trifluoromethylbenzamide,
[44] 2,4-dichloro-N-(6-bromobenzo[d]isoxazol-3-yl)-5-fluorobenzamide,
[45] N-(6-bromobenzo[d]isoxazol-3-yl)-3-fluoro-4-trifluoromethylbenzamide,
[46] N-(6-bromobenzo[d]isoxazol-3-yl)-3-fluoro-5-trifluoromethylbenzamide,
[47] N-(6-bromobenzo[d]isoxazol-3-yl)-2,3,4-trifluorobenzamide,
[48] N-(6-bromobenzo[d]isoxazol-3-yl)-4-propylbenzamide,
[49] N-(6-bromobenzo[d]isoxazol-3-yl)-3,4-difluorobenzamide,
[50] furan-2-(5-bromobenzo[d]isoxazol-3-yl)carboxamide,
[51] 7-fluoro-N-(4-fluorobenzo[d]isoxazol-3-yl)-2-trifluoromethylbenzamide,
[52] 2,4-dichloro-5-fluoro-N-(7-fluorobenzo[d]isoxazol-3-yl)benzamide,
[53] 3-fluoro-N-(7-fluorobenzo[d]isoxazol-3-yl)-4-trifluoromethylbenzamide,
[54] 2,3,4-trifluoro-N-(7-fluorobenzo[d]isoxazol-3-yl)benzamide,
[55] N-(7-fluorobenzo[d]isoxazol-3-yl)-4-iodobenzamide,
[56] 2-chloro-N-(7-fluorobenzo[d]isoxazol-3-yl)nicotinamide,
[57] naphthalene-2-(7-fluorobenzo[d]isoxazol-3-yl)carboxamide,
[58] N-(7-fluorobenzo[d]isoxazol-3-yl)-4-propylbenzamide,
[59] 3,4-difluoro-N-(7-fluorobenzo[d]isoxazol-3-yl)benzamide,
[60] 2-fluoro-N-(7-fluorobenzo[d]isoxazol-3-yl)-3-trifluoromethylbenzamide,
[61] N-(7-fluorobenzo[d]isoxazol-3-yl)-3-methoxybenzamide,
[62] N-(7-fluorobenzo[d]isoxazol-3-yl)-2,2-diphenylacetamide,
[63] furan-2-(7-fluorobenzo[d]isoxazol-3-yl)carboxamide,
[64] 2,4-dichloro-N-(4-dimethylaminobenzo[d]isoxazol-3-yl)-5-fluorobenzamide,
[65] N-(4-dimethylaminobenzo[d]isoxazol-3-yl)-2,3,4-trifluorobenzamide,
[66] 2-chloro-N-(4-dimethylaminobenzo[d]isoxazol-3-yl)nicotinamide,
[67] naphthalene-2-(4-dimethylaminobenzo[d]isoxazol-3-yl)carboxamide,
[68] N-(4-dimethylaminobenzo[d]isoxazol-3-yl)-4-propylbenzamide,
[69] N-(4-dimethylaminobenzo[d]isoxazol-3-yl)-3,4-difluorobenzamide,
[70] N-(4-dimethylaminobenzo[d]isoxazol-3-yl)-2-fluoro-3-trifluoromethylbenzamide,
[71] N-(4-dimethylaminobenzo[d]isoxazol-3-yl)-3-methoxybenzamide,
[72] N-(4-dimethylaminobenzo[d]isoxazol-3-yl)-2,2-dimethylpropionamide,
[73] cyclohexane-(4-dimethylaminobenzo[d]isoxazol-3-yl)carboxamide,
[74] N-(4-dimethylaminobenzo[d]isoxazol-3-yl)-2,2-diphenylacetamide,
[75] furan-2-(4-dimethylaminobenzo[d]isoxazol-3-yl)carboxamide,
[76] N-(4-dimethylaminobenzo[d]isoxazol-3-yl)-2,2,3,3,4,4,4-heptafluorobutyramide,
[77] 4-fluoro-N-[4-(2,2,2-trifluoroethoxy)benzo[d]isoxazol-3-yl]-2-trifluoromethylbenzamide,
[78] 2,4-dichloro-5-fluoro-N-[4-(2,2,2-trifluoroethoxy)benzo[d]isoxazol-3-yl]benzamide,
[79] 3-fluoro-N-[4-(2,2,2-trifluoroethoxy)benzo[d]isoxazol-3-yl]-4-trifluoromethyl-benzamide,
[80] 2,3,4-trifluoro-N-[4-(2,2,2-trifluoroethoxy)benzo[d]isoxazol-3-yl]benzamide,
[81] naphthalene-2-[4-(2,2,2-trifluoroethoxy)benzo[d]isoxazol-3-yl]carboxamide,
[82] 3,4-difluoro-N-[4-(2,2,2-trifluoroethoxy)benzo[d]isoxazol-3-yl]benzamide,
[83] 2-fluoro-N-[4-(2,2,2-trifluoroethoxy)benzo[d]isoxazol-3-yl]-3-trifluoromethyl-benzamide,
[85] N-benzo[d]isoxazol-3-yl-2-ethylhexanamide,
[86] N-benzo[d]isoxazol-3-yl-2-methylbutyramide,
[87] N-benzo[d]isoxazol-3-yl-2,6-difluorobenzamide,
[88] N-benzo[d]isoxazol-3-yl-3-fluoro-4-trifluoromethylbenzamide,
[89] N-benzo[d]isoxazol-3-yl-2,3,6-trifluorobenzamide,
[90] N-benzo[d]isoxazol-3-ylnicotinamide,
[91] 5-methylisoxazole-3-benzo[d]isoxazol-3-ylcarboxamide,
[92] benzo[b]thiophene-3-benzo[d]isoxazol-3-ylcarboxamide,
[93] 2-chloro-N-(7-fluorobenzo[d]isoxazol-3-yl)benzamide,
[94] 4-tert-butyl-N-(7-fluorobenzo[d]isoxazol-3-yl)benzamide,
[95] N-(7-fluorobenzo[d]isoxazol-3-yl)benzamide,
[96] 4-fluoro-N-(7-fluorobenzo[d]isoxazol-3-yl)benzamide,
[97] 2-fluoro-N-(7-fluorobenzo[d]isoxazol-3-yl)benzamide,
[98] N-(7-fluorobenzo[d]isoxazol-3-yl)-2-methoxybenzamide,
[99] N-(7-fluorobenzo[d]isoxazol-3-yl)-2-methylbenzamide,
[100] 3,5-difluoro-N-(6-fluorobenzo[d]isoxazol-3-yl)benzamide,
[101] naphthalene-1-(6-fluorobenzo[d]isoxazol-3-yl)carboxamide,
[102] adamantane-1-(6-fluorobenzo[d]isoxazol-3-yl)carboxamide,
[103] 2-bromo-N-(5-fluorobenzo[d]isoxazol-3-yl)benzamide,
[104] 4-tert-butyl-N-(5-fluorobenzo[d]isoxazol-3-yl)benzamide,
[105] 2,4-difluoro-N-(5-fluorobenzo[d]isoxazol-3-yl)benzamide,
[106] naphthalene-2-(5-fluorobenzo[d]isoxazol-3-yl)carboxamide,
[107] N-(5-fluorobenzo[d]isoxazol-3-yl)-4-propylbenzamide,
[108] 2-chloro-N-(6-chlorobenzo[d]isoxazol-3-yl)benzamide,
[109] 4-tert-butyl-N-(6-chlorobenzo[d]isoxazol-3-yl)benzamide,
[110] N-(6-chlorobenzo[d]isoxazol-3-yl)-4-fluoro-2-trifluoromethylbenzamide,
[111] 2,4-dichloro-N-(6-chlorobenzo[d]isoxazol-3-yl)-5-fluorobenzamide,
[112] N-(6-chlorobenzo[d]isoxazol-3-yl)-2-ethylhexanamide,
[113] N-(6-chlorobenzo[d]isoxazol-3-yl)-2-methylbutyramide,
[114] N-(6-bromobenzo[d]isoxazol-3-yl)-2-chlorobenzamide,
[115] N-(6-bromobenzo[d]isoxazol-3-yl)-3,4-dichlorobenzamide,
[116] thiophene-2-(6-bromobenzo[d]isoxazol-3-yl)carboxamide,
[117] N-(6-bromobenzo[d]isoxazol-3-yl)-3,3-dimethylbutyramide,
[118] N-(5-methylbenzo[d]isoxazol-3-yl)benzamide,
[119] 4-bromo-N-(5-methylbenzo[d]isoxazol-3-yl)benzamide,
[120] 2-chloro-N-(5-methylbenzo[d]isoxazol-3-yl)benzamide,
[121] 4-fluoro-N-(5-methylbenzo[d]isoxazol-3-yl)benzamide,
[122] 2-fluoro-N-(5-methylbenzo[d]isoxazol-3-yl)benzamide,
[123] 3-methyl-N-(5-methylbenzo[d]isoxazol-3-yl)benzamide,
[124] 4-tert-butyl-N-(5-methylbenzo[d]isoxazol-3-yl)benzamide,
[125] 2-methyl-N-(5-methylbenzo[d]isoxazol-3-yl)benzamide,
[126] 2,3-difluoro-N-(5-methylbenzo[d]isoxazol-3-yl)benzamide,
[127] 2,4-difluoro-N-(5-methylbenzo[d]isoxazol-3-yl)benzamide,
[128] 2-chloro-N-(5-methylbenzo[d]isoxazol-3-yl)nicotinamide,
[129] cyclopropane-(5-fluorobenzo[d]isoxazol-3-yl)carboxamide,
[130] N-(5-fluorobenzo[d]isoxazol-3-yl)-3,3-dimethylbutyramide,
[131] 2,4-difluoro-N-(5-fluorobenzo[d]isoxazol-3-yl)benzamide,
[132] 2,5-dimethylfuran-3-(5-fluorobenzo[d]isoxazol-3-yl)carboxamide,
[133] N-(5-bromobenzo[d]isoxazol-3-yl)-2,3-difluorobenzamide,
[134] 2,5-dimethylfuran-3-(5-bromobenzo[d]isoxazol-3-yl)carboxamide,
[135] N-(5-bromobenzo[d]isoxazol-3-yl)-2-methylbutyramide,
[136] N-(5-bromobenzo[d]isoxazol-3-yl)-2,6-difluorobenzamide,
[137] N-(5-bromobenzo[d]isoxazol-3-yl)-3,4-dimethoxybenzamide,
[138] N-(5-bromobenzo[d]isoxazol-3-yl)-4-methylbenzamide,
[139] N-(5-bromobenzo[d]isoxazol-3-yl)-2,6-dimethoxybenzamide,
[140] 2-bromo-N-(5-bromobenzo[d]isoxazol-3-yl)benzamide,
[141] N-(5-bromobenzo[d]isoxazol-3-yl)-5-fluoro-2-trifluoromethylbenzamide,
[142] N-(5-bromobenzo[d]isoxazol-3-yl)-3-methoxybenzamide,
[143] 3-methyl-N-(5-methylbenzo[d]isoxazol-3-yl)benzamide,
[144] 4-cyano-N-(5-methylbenzo[d]isoxazol-3-yl)benzamide,
[145] N-(5-methylbenzo[d]isoxazol-3-yl)-2-phenylbutyramide,
[146] N-(5-methylbenzo[d]isoxazol-3-yl)-2-methylpentanamide,
[147] N-(4-fluorobenzo[d]isoxazol-3-yl)benzamide,
[148] 4-chloro-N-(4-fluorobenzo[d]isoxazol-3-yl)benzamide,
[149] 2-fluoro-N-(4-fluorobenzo[d]isoxazol-3-yl)benzamide,
[150] adamantane-1-(4-fluorobenzo[d]isoxazol-3-yl)carboxamide,
[151] adamantane-1-(4-chlorobenzo[d]isoxazol-3-yl)carboxamide,
[152] N-(4-chlorobenzo[d]isoxazol-3-yl)benzamide,
[153] N-(4-chlorobenzo[d]isoxazol-3-yl)-3-methylbenzamide,
[154] N-(4-chlorobenzo[d]isoxazol-3-yl)-2-methylbutyramide,
[155] N-(4-chlorobenzo[d]isoxazol-3-yl)-3,3-dimethylbutyramide,
[156] N-(4-methoxybenzo[d]isoxazol-3-yl)-2-methylbenzamide,
[157] 2-chloro-N-(4-methoxybenzo[d]isoxazol-3-yl)benzamide,
[158] N-(4-methoxybenzo[d]isoxazol-3-yl)-2-trifluoromethylbenzamide,
[159] N-(4-methoxybenzo[d]isoxazol-3-yl)-2-ethylhexanamide,
[160] N-(4-methoxybenzo[d]isoxazol-3-yl)-2-methylbutyramide,
[161] N-(4-methoxybenzo[d]isoxazol-3-yl)-2-methylpentanamide,
[162]cyclopropane-[4-(2,2,2-trifluoroethoxy)benzo[d]isoxazol-3-yl]carboxamide,
[163] 2-methyl-N-[4-(2,2,2-trifluoroethoxy)benzo[d]isoxazol-3-yl]butyramide,
[164] 3,3-dimethyl-N-[4-(2,2,2-trifluoroethoxy)benzo[d]isoxazol-3-yl]butyramide,
[165]cyclohexane-[4-(2,2,2-trifluoroethoxy)benzo[d]isoxazol-3-yl]carboxamide,
[166] 2,5-dimethylfuran-3-[4-(2,2,2-trifluoroethoxy)benzo[d]isoxazol-3-yl]carboxamide,
[167] N-[4-(4-methylbenzyloxy)benzo[d]isoxazol-3-yl]-3-nitrobenzamide,
[168] N-[4-(4-methylbenzyloxy)benzo[d]isoxazol-3-yl]-4-propylbenzamide,
[169] N-(4-dimethylaminobenzo[d]isoxazol-3-yl)-3-fluoro-5-trifluoromethyl-benzamide,
[170] 3,4-dichloro-N-(4-dimethylaminobenzo[d]isoxazol-3-yl)benzamide,
[171] N-(4-dimethylaminobenzo[d]isoxazol-3-yl)-3-nitrobenzamide,
[172] adamantane-1-(4-dimethylaminobenzo[d]isoxazol-3-yl)carboxamide,
[173] benzo[b]thiophene-2-(4-dimethylaminobenzo[d]isoxazol-3-yl)carboxamide,
[174] N-(4-dimethylaminobenzo[d]isoxazol-3-yl)-3,3-dimethylbutyramide,
[176] N-(6-amino-4,5,7-trifluorobenzo[d]isoxazol-3-yl)-2,6-difluorobenzamide, and
[177] N-(6-amino-4,5,7-trifluorobenzo[d]isoxazol-3-yl)-2,4-difluorobenzamide;
or a pharmaceutically acceptable salt thereof.

49. A method of treating or inhibiting a disorder or disease state selected from the group consisting of pain, eating disorders, migraine; chronic paroxysmal hemicrania, depression, urinary incontinence, cough, asthma, glaucoma, tinnitus, inflammation, neurodegenerative disorders, cognitive dysfunctions or deficiencies, epilepsy, narcolepsy, diarrhea, gastritis, gastric ulcer, pruritus, anxiety, panic attacks, schizophrenia, cerebral ischemia, muscle spasms, cramps, gastroesaphageal reflux syndrome; alcohol, drug or medicament abuse or dependency, or for inhibiting development of tolerance to medicaments or drugs, regulating food intake, modulating motor activity, regulating the cardiovascular system, local anesthesia, increasing vigilance, increasing libido, diuresis, or antinatriuresis in a subject in need thereof, said method comprising administering to said subject a pharmacologically effective amount of a composition according to claim 35.

50. A method according to claim 49, wherein said disorder or disease state is pain.

51. A method according to claim 50, wherein said pain is selected from the group consisting of acute pain, chronic pain, visceral pain and neuropathic pain.

52. A method according to claim 49, wherein said disorder of disease state is an eating disorder selected from the group consisting of bulimia, anorexia, obesity and cachexia; a neurodegenerative disorders selected from the group consisting of Parkinson's disease, Huntington's disease, Alzheimer's disease and multiple sclerosis; memory impairment; attention deficit syndrome (ADS); nicotine dependency; cocaine abuse or dependency; withdrawal symptoms associated with alcohol, drug or medicament abuse or dependency; or development of tolerance to oploids.

53. A substituted N-benzo[d]isoxazol-3-ylamine compound corresponding to formula Ia 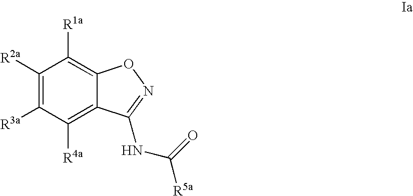
wherein
R^{1a} is H, F, Cl, Br, I, —CN, —NC, —SO₃H, —S(═O₂)NH₂, —OH, —SH, a linear or branched C₁₋₁₀-alkyl group, —OR^{6a}, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{2a} is H, F, Cl, Br, I, —CN, —NC, —SO₃H, —S(═O₂)NH₂, —NH₂, —OH, —SH, a linear or branched C₁₋₁₀ alkyl group, —OR^{6a}, —O—(CH₂)—R^{7a}, —SR^{8a}, —S—(CH₂)—R^{9a} or —NR^{10a}R^{11a};
R^{3a} is H, F, Cl, Br, I, —CN, —NC, —SO₃H, —S(═O₂)NH₂, —OH, —SH, a linear or branched C₁₋₁₀ alkyl group, —OR^{6a}, —O—(CH₂)—R^{7a}, —SR^{8a}, —S—(CH₂)—R^{9a} or —NR^{10a}R^{11a};
R^{4a} is H, F, Cl, Br, I, —CN, —NC, —NO₂, —SO₃H, —S(═O₂)NH₂, —NH₂, —SH, a linear or branched C₁₋₁₀ alkyl group, —OR^{6a}, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{5a} is
a linear or branched C₂₋₁₀ alkyl group;
a —CₘF₂ₘ₊₁ group wherein m is 1, 2, 3, 4 or 5;
a linear or branched C₂₋₁₀ alkenyl group;
an unsaturated or saturated, optionally substituted 3-, 4-, 5-, 6-, 7-, 8- or 9-membered cycloaliphatic group which optionally may be bridged by 1 or 2 linear or branched C₁₋₅-alkylene groups;
an imidazolidinonyl group which optionally may be substituted by 1 or 2 substituents independently selected from the group consisting of methyl, ethyl, n-propyl, isopropyl, n-butyl and tert-butyl;
a phenyl group which optionally may be substituted by 1, 2, 3, 4 or 5 substituents independently selected from the group consisting of F, Cl, Br, I, —CN, —CF₃, —SF₅, —OH, —O—C₁₋₅-alkyl, —O—C₂₋₅-alkenyl, —NH₂, —O—CF₃, —O—CHF₂, —O—CH₂F, —S—CF₃, —S—CHF₂, —S—CH₂F, —SH, —S—C₁₋₁₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl and benzyl; wherein the cyclic part of the groups —S(═O)₂-phenyl, —O-phenyl, —O-benzyl, —S-phenyl, —S-benzyl, phenyl, —(CH₂)-benzo[b]furanyl, dihydro[b]benzofuranyl and benzyl optionally may be substituted by 1, 2, 3, 4 or 5 substituents independently selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl;
with the proviso that the para position or at least one of the meta positions of said phenyl group is substituted by one of the foregoing substituents if the ortho positions of said phenyl group are unsubstituted or substituted by at least one substituent selected from the group consisting of F, Cl, Br, methyl and —O—CH₃, and the groups R¹, R², R³ and R⁴ are each H, or one of the groups R¹, R², R³ and R⁴ is F, Cl, Br, I, —C₁₋₃-alkyl, —CF₃, —C₂F₅, —OCF₃, —OC₂F₅, —CF₂H, —C₂F₄H, —OCF₂H or —OC₂F₄H, and the remainder of the R¹, R², R³ and R⁴ are each H;
a group selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl and 4-methyl-3-nitrophenyl;
an optionally substituted naphthyl group;
an optionally substituted 6- to 10-membered aryl group which is fused to a saturated or unsaturated, optionally substituted mono- or polycyclic ring system;
an optionally substituted 5- to 14-membered heteroaryl group;
—(CH₂)ₙ—C(═O)—OR^{14a}, wherein n is 0, 1, 2, 3, 4 or 5;
—CR^{15a}R^{16a}—O—C(═O)—R^{17a};
—(CHR^{18a})—(CH₂)ₚₐ—R^{19a}, wherein pa is 0 or 1; or
—(CH═CH)—R^{20a};
R^{6a} and R^{8a} each independently are a linear or branched, optionally substituted C₁₋₁₀ alkyl group or an optionally substituted 5- to 14-membered aryl or heteroaryl group;
R^{7a} and R^{9a} each independently are an optionally substituted 5- to 14-membered aryl or heteroaryl group;
R^{10a} and R^{11a} each independently denote hydrogen or a linear or branched C₁₋₁₀ alkyl group;
R^{12a}, R^{13a}, R^{14a} and R^{17a} each independently denote a linear or branched C₁₋₁₀ alkyl group;
R^{15a} is a linear or branched C₁₋₁₀ alkyl group or an optionally substituted 6- to 10-membered aryl group;
R^{16a} and R^{18a} each independently denote hydrogen, a linear or branched C₁₋₁₀ alkyl group, or an optionally substituted 6- to 10-membered aryl group;
R^{19a} is an unsaturated or saturated, optionally substituted 3-, 4-, 5-, 6-, 7-, 8- or 9-membered cycloaliphatic group, an optionally substituted 6- to 10-membered aryl group, or an optionally substituted thiophenyl or furanyl group; and
R^{20a} is an optionally substituted 6- to 10-membered aryl group;
wherein
the aforementioned optionally substituted cycloaliphatic groups may each be unsubstituted or substituted by 1, 2, 3, 4 or 5 substituents independently selected from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—H, —C(═O)—C₁₋₅-alkyl, —C(═O)—O—C₁₋₅-alkyl, —(CH₂)—C(═O)—O—C₁₋₅-alkyl, —NH—C₁₋₅-alkyl, —N(C₁₋₅-alkyl)₂, —(CH₂)-benzo[b]furanyl, —O-phenyl, —O-benzyl, phenyl, benzyl, naphthyl and —(CH₂)-naphthyl; wherein the cyclic part of the groups —O-phenyl, —O-benzyl, phenyl, —(CH₂)-benzo[b]furanyl, benzyl, naphthyl and —(CH₂)-naphthyl may be substituted by 1, 2, 3, 4 or 5 substituents independently selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl;
unless indicated otherwise, the aforementioned optionally substituted aryl, heteroaryl, naphthyl, furanyl or thiophenyl groups may each be unsubstituted or substituted by 1, 2, 3, 4 or 5 substituents independently selected from the group consisting of F, Cl, Br, I, —CN, —CF₃, —SF₅, —OH, —O—C₁₋₅-alkyl, —O—C₂₋₅-alkenyl, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl, —S(═O)₂—C₁₋₅-alkyl, —(CH₂)-benzo[b]furanyl, dihydrobenzo[b]furanyl, —O-phenyl, —O-benzyl, —S-phenyl, —S-benzyl, phenyl, pyridinyl and benzyl; wherein the cyclic part of the groups —S(═O)₂-phenyl, —O-phenyl, —O-benzyl, —S-phenyl, —S-benzyl, phenyl, —(CH₂)-benzo[b]furanyl, dihydro[b]benzofuranyl, pyridinyl and benzyl optionally may be substituted by 1, 2, 3, 4 or 5 substituents independently selected from the group consisting of F, Cl, Br, —OH, —CF₃, —SF₅, —CN, —NO₂, —O—C₁₋₅-alkyl, —C₁₋₅-alkyl, —O—CF₃, —S—CF₃, phenyl and —O-benzyl;
the aforementioned heteroaryl groups each optionally may have 1, 2, 3, 4 or 5 heteroatom(s) independently selected from the group consisting of oxygen, nitrogen and sulfur as ring member(s);
the aforementioned optionally substituted C₁₋₁₀ alkyl groups may each be unsubstituted or optionally each substituted by 1, 2, 3, 4 or 5 substituents independently selected from the group consisting of F, Cl, Br, I, —OH, —SH, —CN and —NO₂;
the rings of the aforementioned optionally substituted mono- or polycyclic ring systems may be unsubstituted or optionally substituted by 1, 2, 3, 4 or 5 substituents independently selected from the group consisting of oxo (═O), thioxo (═S), F, Cl, Br, I, —CN, —CF₃, —SF₅, —OH, —O—C₁₋₅-alkyl, —O—C₂₋₅-alkenyl, —NH₂, —NO₂, —O—CF₃, —O—CHF₂, —O—CH₂F, —S—CF₃, —S—CHF₂, —S—CH₂F, —SH, —S—C₁₋₅-alkyl, —C₁₋₅-alkyl, —C(═O)—O—C₁₋₅-alkyl, —O—C(═O)—C₁₋₅-alkyl, —NH—C₁₋₅-alkyl, —N(C₁₋₅-alkyl)₂, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—NH₂, —S(═O)₂—NH—C₁₋₅-alkyl, —S(═O)₂—N(C₁₋₅-alkyl)₂, —S(═O)₂-phenyl and —S(═O)₂—C₁₋₅-alkyl; and
the rings of the aforementioned mono- or polycyclic ring systems each have 5, 6 or 7 members and may each have 1, 2 or 3 heteroatom(s) as ring member(s) which are independently selected from the group consisting of oxygen, nitrogen and sulfur;
or a salt or solvate thereof.

54. A compound according to claim 53, wherein said compound is in the form of an isolated or purified stereoisomer.

55. A compound according to claim 53, wherein said compound is in the form of a racemic mixture of stereoisomers.

56. A compound according to claim 53, wherein
R^{6a} and R^{8a} each independently denote
a group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CFH₂, —CF₂H, —CBr₃, —CCl₃, —CF₂—CF₃, —CH₂—CF₃, —CH₂—CN, —CH₂—NO₂, —CF₂—CF₂—CF₃, —CH₂—CH₂—CF₃, —CH₂—CH₂—CN, —CH₂—CH₂—NO₂, —CF₂—CF₂—CF₂—CF₃ and —CH₂—CH₂—CH₂—CN; or
a group selected from the group consisting of phenyl, naphthyl, thiophenyl, furanyl and pyridinyl, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

57. A compound according to claim 53, wherein R^{7a} and R^{9a} in each independently denote a group selected from the group consisting of phenyl, naphthyl, thiophenyl, furanyl and pyridinyl, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

58. A compound according to claim 53, wherein R^{10a} and R^{11a} each independently denote hydrogen, or an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

59. A compound according to claim 53, wherein R^{12a}, R^{13a}, R^{14a} and R^{17a} each independently denote an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl.

60. A compound according to claim 53, wherein R^{15a} is
an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl; or
a phenyl or naphthyl group, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

61. A compound according to claim 53, wherein
R^{16a} and R^{18a} in each independently denote
hydrogen;
an alkyl group selected from the group consisting of methyl, ethyl, n-propyl, isopropyl, n-butyl, sec-butyl, isobutyl, tert-butyl, n-pentyl, 2-pentyl, 3-pentyl, neopentyl, n-hexyl, 2-hexyl, 3-hexyl, n-heptyl, 2-heptyl, 3-heptyl, n-octyl, 2-octyl, 3-octyl, n-nonyl, 2-nonyl, 3-nonyl and 3,5,5-trimethylhexyl; or
a phenyl or naphthyl group, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

62. A compound according to claim 53, wherein R^{19a} is
a cycloaliphatic group selected from the group consisting of cyclopropyl, cyclobutyl, cyclopentyl, cyclohexyl, cycloheptyl, cyclopentenyl, cyclohexenyl and cycloheptenyl, wherein said cycloaliphatic group optionally may be substituted by 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —O—C₃H₇, —NH₂, —NO₂, —O—CF₃, —S—CF₃, —SH, —S—CH₃, —S—C₂H₅, —S—C₃H₇, methyl, ethyl, n-propyl, isopropyl, n-butyl, sec-butyl, isobutyl, tert-butyl, n-pentyl, 2-pentyl, 3-pentyl, neopentyl, —C(═O)—H, —C(═O)—CH₃, —C(═O)—C₂H₅, —C(═O)—O—CH₃, —C(═O)—C₂H₅, —C(═O)—C(CH₃)₃, —NH—CH₃, —NH—C₂H₅, —N(CH₃)₂, —N(C₂H₅)₂ and —N(CH(CH₃)₂)₂; or
a group selected from the group consisting of phenyl, naphthyl, furanyl and thiophenyl, wherein said group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

63. A compound according to claim 53, wherein R^{20a} is a phenyl or naphthyl group, wherein said phenyl or naphthyl group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl and tert-butyl.

64. A compound according to claim 53, wherein
R^{1a} is H, F, Cl, Br, I, —CN, —NC, —SO₃H, —S(═O₂)NH₂, —OH, —SH, methyl, ethyl, n-propyl, isopropyl, n-butyl, sec-butyl, isobutyl, tert-butyl, n-pentyl, sec-pentyl, n-hexyl, —OR^{6a}, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{2a} is H, F, Cl, Br, I, —CN, —NC, —SO₃H, —S(═O₂)NH₂, —NH₂, —OH, —SH, methyl, ethyl, n-propyl, isopropyl, n-butyl, sec-butyl, isobutyl, tert-butyl, n-pentyl, sec-pentyl, n-hexyl, —OR^{6a}, —O—(CH₂)—R^{7a}, —SR^{8a}, —S—(CH₂)—R^{9a} or —NR^{10a}R^{11a};
R^{3a} is H, F, Cl, Br, I, —CN, —NC, —SO₃H, —S(═O₂)NH₂, —OH, —SH, methyl, ethyl, n-propyl, isopropyl, n-butyl, sec-butyl, isobutyl, tert-butyl, n-pentyl, sec-pentyl, n-hexyl, —OR^{6a}, —O—(CH₂)—R^{7a}, —SR^{8a}, —S—(CH₂)—R^{9a} or —NR^{10a}R^{11a};
R^{4a} is H, F, Cl, Br, I, —CN, —NC, —NO₂, —SO₃H, —S(═O₂)NH₂, —NH₂, —SH, methyl, ethyl, n-propyl, isopropyl, n-butyl, sec-butyl, isobutyl, tert-butyl, n-pentyl, sec-pentyl, n-hexyl, —OR^{6a}, —O—(CH₂)—R^{7a}, —SR^{8a}, —S—(CH₂)—R^{9a}, —NR^{10a}R^{11a}, —NH—C(═O)—R^{12a} or —NR^{13a}—C(═O)—R^{12a};
R^{5a} is
a group selected from the group consisting of ethyl, n-propyl, isopropyl, n-butyl, sec-butyl, isobutyl, tert-butyl, n-pentyl, 2-pentyl, 3-pentyl, neopentyl, n-hexyl, 3-heptyl, 3-octyl, 3,5,5-trimethylhexyl, —CF₃, —CF₂—CF₃, —CF₂—CF₂—CF₃, —CF₂—CF₂—CF₂—CF₃, 1-propenyl, 2-propenyl, 1-butenyl, 2-butenyl, 3-butenyl and 2-methyl-1-propenyl;
a cycloaliphatic group selected from the group consisting of cyclopropyl, cyclobutyl, cyclopentyl, cyclohexyl, cycloheptyl, cyclopentenyl, cyclohexenyl, cycloheptenyl, [6,6]-dimethyl-[3.1.1]-bicycloheptyl and adamantly; wherein the cycloaliphatic group optionally may be substituted by 1, 2, 3, 4 or 5 substituents independently selected from the group consisting of oxo (═O), thioxo (═S), F, Cl, Br, I, —CN, —CF₃, —SF₅, —O—CH₃, —O—C₂H₅, —O—C₃H₇, methyl, ethyl, n-propyl, isopropyl, n-butyl, sec-butyl, isobutyl, tert-butyl, phenyl and benzyl, wherein the cyclic part of the groups phenyl and benzyl may be substituted by 1, 2, 3, 4 or 5 substituents independently selected from the group consisting of F, Cl, Br, methyl, ethyl and n-propyl;
an imidazolidinonyl group;
a phenyl group optionally substituted by 1, 2, 3, 4 or 5 substituents independently selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —NH—CH₃, —NH—C₂H₅, —N(CH₃)₂, —N(C₂H₅)₂, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, phenyl and benzyl; wherein the cyclic part of the phenyl and benzyl groups optionally may be substituted by 1, 2, 3, 4 or 5 substituents independently selected from the group consisting of F, Cl, Br, —OH, —CF₃, —SF₅, —CN, —NO₂, —O—CH₃, —O—C₂H₅, —O—C₃H₇ methyl, ethyl, —O—CF₃ and —S—CF₃;
a group selected from the group consisting of 4-nitrophenyl, 3-nitrophenyl, 2-nitrophenyl, 4-chloro-3-nitrophenyl, 4-fluoro-3-nitrophenyl, 4-bromo-3-nitrophenyl, (3,5)-dinitrophenyl and 4-methyl-3-nitrophenyl;
a group selected from the group consisting of naphthyl, [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl, wherein said group optionally may be substituted by 1, 2, 3, 4 or 5 substituents independently selected from the group consisting of F, Cl, Br, I, —CN, —CF₃, —O—CH₃, —O—C₂H₅, —O—C₃H₇, —O—CF₃, —O—CHF₂, —O—CH₂F, —S—CF₃, —S—CHF₂, —S—CH₂F, —S—CH₃, —S—C₂H₅, —S—C₃H₇, methyl, ethyl, n-propyl, isopropyl, n-butyl, sec-butyl, isobutyl and tert-butyl;
a heteroaryl group selected from the group consisting of thiophenyl, furanyl, pyrazolyl, pyrazinyl, pyranyl, triazolyl, pyridinyl, indolyl, benzo[b]furanyl, benzo[b]thiophenyl, thiazolyl, oxazolyl, isoxazolyl, indazolyl, quinoxalinyl, quinolinyl, isoquinolinyl, benzo[2,1,3]thiadiazolyl, [1,2,3]-benzothiadiazolyl, [2,1,3]-benzoxadiazolyl and [1,2,3]-benzoxadiazolyl; wherein said heteroaryl group optionally may be substituted by 1, 2, 3, 4 or 5 substituents independently selected from the group consisting of F, Cl, Br, I, —CN, —CF₃, —SF₅, —OH, —O—CH₃, —O—C₂H₅, —O—C₃H₇, —O—CH₂—CH═CH₂, —NO₂, —O—CF₃, —O—CHF₂, —O—CH₂F, —S—CF₃, —S—CHF₂, —S—CH₂F, —SH, —S—CH₃, —S—C₂H₅, —S—C₃H₇, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —S(═O)₂—NH₂, —S(═O)₂—NH—CH₃, —S(═O)₂—N(CH₃)₂, —S(═O)₂—N(C₂H₅)₂, —S(═O)₂—N(n-C₃H₇)₂, —S(═O)₂—N(CH(CH₃)₂)₂, —S(═O)₂-phenyl, —S(═O)₂—CH₃, —S(═O)₂—C₂H₅, —S(═O)₂—CH(CH₃)₂, dihydrobenzo[b]furanyl, —O-phenyl, —O-benzyl, —S-phenyl, —S-benzyl, phenyl, pyridinyl and benzyl; wherein the cyclic part of the groups —S(═O)₂-phenyl, —O-phenyl, —O-benzyl, —S-phenyl, —S-benzyl, phenyl, dihydro[b]benzofuranyl, pyridinyl and benzyl optionally may be substituted by 1, 2, 3, 4 or 5 substituents independently selected from the group consisting of F, Cl, Br, —CF₃, —CN, —NO₂, —O—CH₃, —O—C₂H₅, —O—C₃H₇, methyl, ethyl, n-propyl, isopropyl, n-butyl, sec-butyl, isobutyl, tert-butyl, —O—CF₃ and —S—CF₃;
—(CH₂)ₙ—C(═O)—OR^{14a}, wherein n is 0, 1, 2, 3 or 4;
—CR^{15a}R^{16a}—O—C(═O)—R^{17a};
—(CHR^{18a})—(CH₂)ₚₐ—R^{19a}, wherein pa is 0 or 1; or
—(CH═CH)—R^{20a};
R^{6a} and R^{8a} each independently denote a group selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R^{7a} and R^{9a} each independently denote a phenyl group optionally substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
R^{10a} and R^{11a} in each independently denote an alkyl group selected from the group consisting of methyl, ethyl, n-propyl, isopropyl, n-butyl, isobutyl and tert-butyl;
R^{12a}, R^{13a}, R^{14a} and R^{17a} each independently denote an alkyl group selected from the group consisting of methyl, ethyl, n-propyl, isopropyl and tert-butyl;
R^{15a} is an alkyl group selected from the group consisting of methyl, ethyl, n-propyl, isopropyl and n-butyl, or a phenyl group;
R^{16a} is hydrogen, or an alkyl group selected from the group consisting of methyl, ethyl, n-propyl, isopropyl, n-butyl, isobutyl and tert-butyl;
R^{18a} is hydrogen; an alkyl group selected from the group consisting of methyl, ethyl, n-propyl, isopropyl, n-butyl, isobutyl and tert-butyl; or a phenyl group;
R^{19a} is
a cycloaliphatic group selected from the group consisting of cyclopropyl, cyclobutyl, cyclopentyl, cyclohexyl, cycloheptyl, cyclopentenyl, cyclohexenyl and cycloheptenyl;
a phenyl group which may be substituted by 1, 2, 3, 4 or 5 substituents independently selected from the group consisting of F, Cl, Br, I, —CN, —CF₃, —SF₅, —O—CH₃, —O—C₂H₅, —O—C₃H₇, —NO₂, —O—CF₃, —O—CHF₂, —O—CH₂F, methyl, ethyl, n-propyl, isopropyl, n-butyl, sec-butyl, isobutyl and tert-butyl; or
a thiophenyl group; and
R^{20a} is a phenyl group optionally substituted by 1, 2, 3, 4 or 5 substituents independently selected from the group consisting of F, Cl, Br, —CF₃, methyl, ethyl, n-propyl, isopropyl, n-butyl, sec-butyl, isobutyl and tert-butyl.

65. A compound according to claim 53, wherein
R^{1a} is H, F, Cl or Br;
R^{2a} is H, F, Cl, Br or —NH₂;
R^{3a} is H, F, Cl, Br, methyl, ethyl or n-propyl;
R^{4a} is H, F, Cl, Br, —NH₂, methyl, ethyl, n-propyl, isopropyl, n-butyl, isobutyl, tert-butyl, —OR^{6a}, —O—(CH₂)—R^{7a} or —NR^{10a}R^{11a};
R^{5a} is
a group selected from the group consisting of ethyl, n-propyl, isopropyl, n-butyl, sec-butyl, isobutyl, tert-butyl, n-pentyl, 2-pentyl, 3-pentyl, neopentyl, n-hexyl, 3-heptyl, —CF₃, —CF₂—CF₃, —CF₂—CF₂—CF₃ and —CF₂—CF₂—CF₂—CF₃;
a cycloaliphatic group selected from the group consisting of cyclopropyl, cyclobutyl, cyclopentyl, cyclohexyl, cycloheptyl, cyclopentenyl, cyclohexenyl, cycloheptenyl, (6,6)-dimethyl-[3.1.1]-bicycloheptyl and adamantyl;
a group selected from the group consisting of 2-trifluoromethyl-phenyl, 2-nitrophenyl, 2-dimethylaminophenyl, 2-diethylaminophenyl, 2-aminophenyl, 2-ethylphenyl, 2-methylbenzoate, 2-bromophenyl, 2-chlorophenyl, 2-fluorophenyl, 2-methylphenyl, 2-methoxyphenyl, 2-ethoxyphenyl, 2-cyanophenyl, 2-acetylphenyl, 2-dimethylamino-sulfonylphenyl, 3-chlorophenyl, 3-methylphenyl, 3-nitrophenyl, 3-trifluoromethylphenyl, 3-fluorophenyl, 3-bromophenyl, 3-dimethylaminophenyl, 3-diethylaminophenyl, 3-aminophenyl, 3-methoxyphenyl, 3-ethylphenyl, 3-ethoxyphenyl, 3-cyanophenyl, 3-acetylphenyl, 3-phenylphenyl, 3-dimethylaminosulfonylphenyl, 4-bromophenyl, 4-methoxyphenyl, 4-chlorophenyl, 4-fluorophenyl, 4-tert-butylphenyl, 4-cyanophenyl, 4-nitrophenyl, 4-methylphenyl, 4-phenylphenyl, 4-trifluoromethylphenyl, 4-dimethylaminophenyl, 4-diethylaminophenyl, 4-aminophenyl, 4-iodophenyl, 4-n-propylphenyl, 4-di-n-propylaminosulfonylphenyl, 4-diethylaminosulfonylphenyl, 4-dimethylaminosulfonylphenyl, 4-ethylphenyl, 4-ethoxyphenyl, 4-methylbenzoate, 4-acetylphenyl, 2-fluoro-3-trifluoromethylphenyl, (2,3)-difluorophenyl, (2,3)-dimethylphenyl, (2,3)-dichlorophenyl, 3-fluoro-2-trifluoromethylphenyl, (2,4)-dichlorophenyl, (2,4)-difluorophenyl, 4-fluoro-2-trifluoromethylphenyl, (2,4)-dimethoxyphenyl, 2-chloro-4-fluorophenyl, (2,4)-dibromophenyl, 2-fluoro-4-trifluoromethylphenyl, (2,5)-difluorophenyl, 2-fluoro-5-trifluoromethylphenyl, 5-fluoro-2-trifluoromethylphenyl, 5-chloro-2-trifluoromethylphenyl, 5-bromo-2-trifluoromethylphenyl, (2,5)-dimethoxyphenyl, (2,5)-bis-trifluoromethylphenyl, (2,5)-dichlorophenyl, (2,5)-dibromophenyl, 2-fluoro-6-trifluoromethylphenyl, (2,6)-dimethoxyphenyl, (2,6)-dimethylphenyl, 2-chloro-6-fluorophenyl, 2-bromo-6-chlorophenyl, 2-bromo-6-fluorophenyl, (2,6)-difluorophenyl, (2,6)-dibromophenyl, (2,6)-dichlorophenyl, (3,4)-dichlorophenyl, 4-chloro-3-nitrophenyl, (3,4)-dimethoxyphenyl, 4-fluoro-3-trifluoromethylphenyl, 3-fluoro-4-trifluoromethylphenyl, (3,4)-difluorophenyl, 4-bromo-3-methylphenyl, 4-bromo-5-methylphenyl, 3-chloro-4-fluorophenyl, 4-fluoro-3-nitrophenyl, 4-bromo-3-nitrophenyl, (3,4)-dibromophenyl, 4-chloro-3-methylphenyl, 4-fluoro-3-methylphenyl, 4-methyl-3-nitrophenyl, (3,5)-dimethoxyphenyl, (3,5)-bis-trifluoromethylphenyl, (3,5)-difluorophenyl, (3,5)-dinitrophenyl, (3,5)-dichlorophenyl, 3-fluoro-5-trifluoromethylphenyl, 5-fluoro-3-trifluoromethylphenyl, (3,5)-dibromophenyl, 5-chloro-4-fluorophenyl, 5-chloro-4-fluorophenyl, 5-bromo-4-methylphenyl, (2,3,4)-trifluorophenyl, (2,3,4)-trichlorophenyl, (2,3,6)-trifluorophenyl, (2,4,6)-trichlorophenyl, (2,4,5)-trifluorophenyl, (2,4,5)-trichlorophenyl, (2,4)-dichloro-5-fluorophenyl, (2,4,6)-trichlorophenyl, (2,4,6)-trimethylphenyl, (2,4,6)-trifluorophenyl, (2,4,6)-trimethoxyphenyl, (3,4,5)-trimethoxyphenyl, (2,3,4,5)-tetrafluorophenyl and (2,3,4,5,6)-pentafluorophenyl;
a naphthyl group;
a heteroaryl group selected from the group consisting of thiophenyl, furanyl, pyridinyl, benzo[b]furanyl, benzo[b]thiophenyl, oxazolyl and isoxazolyl; wherein said heteroaryl group optionally may be substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
—(CH₂)ₙ—C(═O)—OR^{14a}, wherein n is 1 or 2; or
—(CHR^{18a})—(CH₂)ₚₐ—R^{19a}, wherein p is 0 or 1;
R^{6a} is a group selected from the group consisting of methyl, ethyl, n-propyl, n-butyl, isobutyl, tert-butyl, —CF₃, —CFH₂, —CF₂H, —CF₂—CF₃, —CH₂—CF₃ and —CF₂—CF₂—CF₃;
R^{7a} is a phenyl group optionally substituted by 1, 2, 3, 4 or 5 substituents independently selected from the group consisting of F, Cl, Br, methyl, ethyl, n-propyl, isopropyl, n-butyl, sec-butyl, isobutyl and tert-butyl;
R^{10a} and R^{11a} each independently denote an alkyl group selected from the group consisting of methyl, ethyl, n-propyl, isopropyl, n-butyl, isobutyl and tert-butyl;
R^{14a} is an alkyl group selected from the group consisting of methyl, ethyl, n-propyl, isopropyl, n-butyl, isobutyl and tert-butyl;
R^{18a} is hydrogen; an alkyl group selected from the group consisting of methyl, ethyl, n-propyl, isopropyl, n-butyl, isobutyl and tert-butyl; or a phenyl group; and
R^{19a} is
a cycloaliphatic group selected from the group consisting of cyclopropyl, cyclobutyl, cyclopentyl, cyclohexyl, cycloheptyl, cyclopentenyl, cyclohexenyl and cycloheptenyl;
a phenyl group which may be substituted by 1, 2, 3, 4 or 5 substituents independently selected from the group consisting of F, Cl, Br, I, —CN, —CF₃, —SF₅, —O—CH₃, —O—C₂H₅, —O—C₃H₇, —NO₂, —O—CF₃, —O—CHF₂, —O—CH₂F, methyl, ethyl, n-propyl, isopropyl, n-butyl, sec-butyl, isobutyl and tert-butyl; or
a thiophenyl group.

66. A compound according to claim 53, selected from the group consisting of:
[1] thiophene-2-benzo[d]isoxazol-3-ylcarboxamide,
[2] N-benzo[d]isoxazol-3-ylsuccinic acid methyl ester
[3] naphthalene-2-benzo[d]isoxazol-3-ylcarboxamide,
[4] adamantane-2-benzo[d]isoxazol-3-ylcarboxamide,
[5] cyclohexane-benzo[d]isoxazol-3-ylcarboxamide,
[6] N-benzo[d]isoxazol-3-yl-2,2-dimethylpropionamide,
[7] N-(4-methoxybenzo[d]isoxazol-3-yl)-2,2-diphenylacetamide,
[8] cyclopropane(5-bromobenzo[d]isoxazol-3-yl)carboxamide,
[9] 3,5-dichloro-N-(6-fluorobenzo[d]isoxazol-3-yl)benzamide,
[10] N-(4-aminobenzo[d]isoxazol-3-yl)-4-nitrobenzamide,
[11] naphthalene-1-(4-aminobenzo[d]isoxazol-3-yl)carboxamide,
[12] benzo[b]thiophene-2-(4-fluorobenzo[d]isoxazol-3-yl)carboxamide,
[13] N-benzo[d]isoxazol-3-yl-2-trifluoromethylbenzamide,
[14] N-benzo[d]isoxazol-3-yl-3,4-dichlorobenzamide
[15] N-benzo[d]isoxazol-3-yl-2,3-difluorobenzamide,
[16] N-(4-dimethylaminobenzo[d]isoxazol-3-yl)-3-fluorobenzamide,
[17] thiophene-2-(6-fluorobenzo[d]isoxazol-3-yl)carboxamide,
[18] N-(6-fluorobenzo[d]isoxazol-3-yl)-2-methylbutyramide,
[19] N-(6-chlorobenzo[d]isoxazol-3-yl)-2-fluoro-3-trifluoromethylbenzamide,
[20] N-(6-chlorobenzo[d]isoxazol-3-yl)-3-methoxybenzamide,
[21] N-(6-bromobenzo[d]isoxazol-3-yl)-4-tert-butylbenzamide,
[23] N-(6-bromobenzo[d]isoxazol-3-yl)-2-trifluoromethylbenzamide,
[24] adamantane-2-(6-bromobenzo[d]isoxazol-3-yl)carboxamide,
[25] N-(6-bromobenzo[d]isoxazol-3-yl)-2-methylbutyramide,
[26] N-(5-fluorobenzo[d]isoxazol-3-yl)-2-trifluoromethylbenzamide,
[27] 3,4-dichloro-N-(5-fluorobenzo[d]isoxazol-3-yl)benzamide,
[29] N-(5-bromobenzo[d]isoxazol-3-yl)-2-fluoro-5-trifluoromethylbenzamide,
[30] N-(5-bromobenzo[d]isoxazol-3-yl)-2,4-difluorobenzamide,
[31] N-(5-methylbenzo[d]isoxazol-3-yl)-2-trifluoromethylbenzamide,
[32] 3-fluoro-N-(5-methylbenzo[d]isoxazol-3-yl)benzamide,
[33] N-(4-methoxybenzo[d]isoxazol-3-yl)-3,5-bis-trifluoromethylbenzamide,
[34] 3,5-difluoro-N-(4-methoxybenzo[d]isoxazol-3-yl)benzamide,
[35] N-(4-dimethylaminobenzo[d]isoxazol-3-yl)-3,5-bis-trifluoromethylbenzamide,
[36] 2-bromo-N-[4-(2,2,2-trifluoroethoxy)benzo[d]isoxazol-3-yl]benzamide,
[37] 3-chloro-N-[4-(2,2,2-trifluoroethoxy)benzo[d]isoxazol-3-yl]benzamide,
[38] 4-tert-butyl-N-[4-(2,2,2-trifluoroethoxy)benzo[d]isoxazol-3-yl]benzamide,
[39] 2-methoxy-N-[4-(2,2,2-trifluoroethoxy)benzo[d]isoxazol-3-yl]benzamide,
[40] N-(6-chlorobenzo[d]isoxazol-3-yl)-4-iodobenzamide,
[41] naphthalene-2-(6-chlorobenzo[d]isoxazol-3-yl)carboxamide,
[42] N-(6-chlorobenzo[d]isoxazol-3-yl)-3,4-difluorobenzamide,
[43] N-(6-bromobenzo[d]isoxazol-3-yl)-4-fluoro-2-trifluoromethylbenzamide,
[44] 2,4-dichloro-N-(6-bromobenzo[d]isoxazol-3-yl)-5-fluorobenzamide,
[45] N-(6-bromobenzo[d]isoxazol-3-yl)-3-fluoro-4-trifluoromethylbenzamide,
[46] N-(6-bromobenzo[d]isoxazol-3-yl)-3-fluoro-5-trifluoromethylbenzamide,
[47] N-(6-bromobenzo[d]isoxazol-3-yl)-2,3,4-trifluorobenzamide,
[48] N-(6-bromobenzo[d]isoxazol-3-yl)-4-propylbenzamide,
[49] N-(6-bromobenzo[d]isoxazol-3-yl)-3,4-difluorobenzamide,
[50] furan-2-(5-bromobenzo[d]isoxazol-3-yl)carboxamide,
[51] 7-fluoro-N-(4-fluorobenzo[d]isoxazol-3-yl)-2-trifluoromethylbenzamide,
[52] 2,4-dichloro-5-fluoro-N-(7-fluorobenzo[d]isoxazol-3-yl)benzamide,
[53] 3-fluoro-N-(7-fluorobenzo[d]isoxazol-3-yl)-4-trifluoromethylbenzamide,
[54] 2,3,4-trifluoro-N-(7-fluorobenzo[d]isoxazol-3-yl)benzamide,
[55] N-(7-fluorobenzo[d]isoxazol-3-yl)-4-iodobenzamide,
[56] 2-chloro-N-(7-fluorobenzo[d]isoxazol-3-yl)nicotinamide,
[57] naphthalene-2-(7-fluorobenzo[d]isoxazol-3-yl)carboxamide,
[58] N-(4-fluorobenzo[d]isoxazol-3-yl)-74-propylbenzamide,
[59] 3,4-difluoro-N-(7-fluorobenzo[d]isoxazol-3-yl)benzamide,
[60] 2-fluoro-N-(7-fluorobenzo[d]isoxazol-3-yl)-3-trifluoromethylbenzamide,
[61] N-(7-fluorobenzo[d]isoxazol-3-yl)-3-methoxybenzamide,
[62] N-(7-fluorobenzo[d]isoxazol-3-yl)-2,2-diphenylacetamide,
[63] furan-2-(7-fluorobenzo[d]isoxazol-3-yl)carboxamide,
[64] 2,4-dichloro-N-(4-dimethylaminobenzo[d]isoxazol-3-yl)-5-fluorobenzamide,
[65] N-(4-dimethylaminobenzo[d]isoxazol-3-yl)-2,3,4-trifluorobenzamide,
[66] 2-chloro-N-(4-dimethylaminobenzo[d]isoxazol-3-yl)nicotinamide,
[67] naphthalene-2-(4-dimethylaminobenzo[d]isoxazol-3-yl)carboxamide,
[68] N-(4-dimethylaminobenzo[d]isoxazol-3-yl)-4-propylbenzamide,
[69] N-(4-dimethylaminobenzo[d]isoxazol-3-yl)-3,4-difluorobenzamide,
[70] N-(4-dimethylaminobenzo[d]isoxazol-3-yl)-2-fluoro-3-trifluoromethylbenzamide,
[71] N-(4-dimethylaminobenzo[d]isoxazol-3-yl)-3-methoxybenzamide,
[72] N-(4-dimethylaminobenzo[d]isoxazol-3-yl)-2,2-dimethylpropionamide,
[73] cyclohexane-(4-dimethylaminobenzo[d]isoxazol-3-yl)carboxamide,
[74] N-(4-dimethylaminobenzo[d]isoxazol-3-yl)-2,2-diphenylacetamide,
[75] furan-2-(4-dimethylaminobenzo[d]isoxazol-3-yl)carboxamide,
[76] N-(4-dimethylaminobenzo[d]isoxazol-3-yl)-2,2,3,3,4,4,4-heptafluorobutyramide,
[77] 4-fluoro-N-[4-(2,2,2-trifluoroethoxy)benzo[d]isoxazol-3-yl]-2-trifluoromethylbenzamide,
[78] 2,4-dichloro-5-fluoro-N-[4-(2,2,2-trifluoroethoxy)benzo[d]isoxazol-3-yl]benzamide,
[79] 3-fluoro-N-[4-(2,2,2-trifluoroethoxy)benzo[d]isoxazol-3-yl]-4-trifluoromethyl-benzamide,
[80] 2,3,4-trifluoro-N-[4-(2,2,2-trifluoroethoxy)benzo[d]isoxazol-3-yl]benzamide,
[81] naphthalene-2-[4-(2,2,2-trifluoroethoxy)benzo[d]isoxazol-3-yl]carboxamide,
[82] 3,4-difluoro-N-[4-(2,2,2-trifluoroethoxy)benzo[d]isoxazol-3-yl]benzamide,
[83] 2-fluoro-N-[4-(2,2,2-trifluoroethoxy)benzo[d]isoxazol-3-yl]-3-trifluoromethyl-benzamide,
[85] N-benzo[d]isoxazol-3-yl-2-ethylhexanamide,
[86] N-benzo[d]isoxazol-3-yl-2-methylbutyramide,
[88] N-benzo[d]isoxazol-3-yl-3-fluoro-4-trifluoromethylbenzamide,
[89] N-benzo[d]isoxazol-3-yl-2,3,6-trifluorobenzamide,
[90] N-benzo[d]isoxazol-3-ylnicotinamide,
[91] 5-methylisoxazol-3-benzo[d]isoxazol-3-ylcarboxamide,
[92] benzo[b]thiophene-3-benzo[d]isoxazol-3-ylcarboxamide,
[94] 4-tert-butyl-N-(7-fluorobenzo[d]isoxazol-3-yl)benzamide,
[96] 4-fluoro-N-(7-fluorobenzo[d]isoxazol-3-yl)benzamide,
[100] 3,5-difluoro-N-(6-fluorobenzo[d]isoxazol-3-yl)benzamide,
[101] naphthalene-1-(6-fluorobenzo[d]isoxazol-3-yl)carboxamide,
[102] adamantane-1-(6-fluorobenzo[d]isoxazol-3-yl)carboxamide,
[104] 4-tert-butyl-N-(5-fluorobenzo[d]isoxazol-3-yl)benzamide,
[105] 2,4-difluoro-N-(5-fluorobenzo[d]isoxazol-3-yl)benzamide,
[106] naphthalene-2-(5-fluorobenzo[d]isoxazol-3-yl)carboxamide,
[107] N-(5-fluorobenzo[d]isoxazol-3-yl)-4-propylbenzamide,
[109] 4-tert-butyl-N-(6-chlorobenzo[d]isoxazol-3-yl)benzamide,
[110] N-(6-chlorobenzo[d]isoxazol-3-yl)-4-fluoro-2-trifluoromethylbenzamide,
[111] 2,4-dichloro-N-(6-chlorobenzo[d]isoxazol-3-yl)-5-fluorobenzamide,
[112] N-(6-chlorobenzo[d]isoxazol-3-yl)-2-ethylhexanamide,
[113] N-(6-chlorobenzo[d]isoxazol-3-yl)-2-methylbutyramide,
[115] N-(6-bromobenzo[d]isoxazol-3-yl)-3,4-dichlorobenzamide,
[116] thiophene-2-(6-bromobenzo[d]isoxazol-3-yl)carboxamide,
[117] N-(6-bromobenzo[d]isoxazol-3-yl)-3,3-dimethylbutyramide,
[119] 4-bromo-N-(5-methylbenzo[d]isoxazol-3-yl)benzamide,
[121] 4-fluoro-N-(5-methylbenzo[d]isoxazol-3-yl)benzamide,
[123] 3-methyl-N-(5-methylbenzo[d]isoxazol-3-yl)benzamide,
[124] 4-tert-butyl-N-(5-methylbenzo[d]isoxazol-3-yl)benzamide,
[126] 2,3-difluoro-N-(5-methylbenzo[d]isoxazol-3-yl)benzamide,
[127] 2,4-difluoro-N-(5-methylbenzo[d]isoxazol-3-yl)benzamide,
[128] 2-chloro-N-(5-methylbenzo[d]isoxazol-3-yl)nicotinamide,
[129] cyclopropane-(5-fluorobenzo[d]isoxazol-3-yl)carboxamide,
[130] N-(5-fluorobenzo[d]isoxazol-3-yl)-3,3-dimethylbutyramide,
[131] 2,4-difluoro-N-(5-fluorobenzo[d]isoxazol-3-yl)benzamide,
[132] 2,5-dimethylfuran-3-(5-fluorobenzo[d]isoxazol-3-yl)carboxamide,
[133] N-(5-bromobenzo[d]isoxazol-3-yl)-2,3-difluorobenzamide,
[134] 2,5-dimethylfuran-3-(5-bromobenzo[d]isoxazol-3-yl)carboxamide,
[135] N-(5-bromobenzo[d]isoxazol-3-yl)-2-methylbutyramide,
[137] N-(5-bromobenzo[d]isoxazol-3-yl)-3,4-dimethoxybenzamide,
[138] N-(5-bromobenzo[d]isoxazol-3-yl)-4-methylbenzamide,
[141] N-(5-bromobenzo[d]isoxazol-3-yl)-5-fluoro-2-trifluoromethylbenzamide,
[142] N-(5-bromobenzo[d]isoxazol-3-yl)-3-methoxybenzamide,
[143] 3-methyl-N-(5-methylbenzo[d]isoxazol-3-yl)benzamide,
[144] 4 -cyano-N-(5-methylbenzo[d]isoxazol-3-yl)benzamide,
[145] N-(5-methylbenzo[d]isoxazol-3-yl)-2-phenylbutyramide,
[146] N-(5-methylbenzo[d]isoxazol-3-yl)-2-methylpentanamide,
[147] 4-chloro-N-(4-fluorobenzo[d]isoxazol-3-yl)benzamide,
[150] adamantane-1-(4-fluorobenzo[d]isoxazol-3-yl)carboxamide,
[151] adamantane-1-(4-chlorobenzo[d]isoxazol-3-yl)carboxamide,
[153] N-(4-chlorobenzo[d]isoxazol-3-yl)-3-methylbenzamide,
[154] N-(4-chlorobenzo[d]isoxazol-3-yl)-2-methylbutyramide,
[155] N-(4-chlorobenzo[d]isoxazol-3-yl)-3,3-dimethylbutyramide,
[156] N-(4-methoxybenzo[d]isoxazol-3-yl)-2-methylbenzamide,
[157] 2-chloro-N-(4-methoxybenzo[d]isoxazol-3-yl)benzamide,
[158] N-(4-methoxybenzo[d]isoxazol-3-yl)-2-trifluoromethylbenzamide,
[159] N-(4-methoxybenzo[d]isoxazol-3-yl)-2-ethylhexanamide,
[160] N-(4-methoxybenzo[d]isoxazol-3-yl)-2-methylbutyramide,
[161] N-(4-methoxybenzo[d]isoxazol-3-yl)-2-methylpentanamide,
[162] cyclopropane-[4-(2,2,2-trifluoroethoxy)benzo[d]isoxazol-3-yl]carboxamide,
[163] 2-methyl-N-[4-(2,2,2-trifluoroethoxy)benzo[d]isoxazol-3-yl]butyramide,
[164] 3,3-dimethyl-N-[4-(2,2,2-trifluoroethoxy)benzo[d]isoxazol-3-yl]butyramide,
[165] cyclohexane-[4-(2,2,2-trifluoroethoxy)benzo[d]isoxazol-3-yl]carboxamide,
[166] 2,5-dimethylfuran-3-[4-(2,2,2-trifluoroethoxy)benzo[d]isoxazol-3-yl]carboxamide,
[167] N-[4-(4-methylbenzyloxy)benzo[d]isoxazol-3-yl]-3-nitrobenzamide,
[168] N-[4-(4-methylbenzyloxy)benzo[d]isoxazol-3-yl]-4-propylbenzamide,
[169] N-(4-dimethylaminobenzo[d]isoxazol-3-yl)-3-fluoro-5-trifluoromethylbenzamide,
[170] 3,4-dichloro-N-(4-dimethylaminobenzo[d]isoxazol-3-yl)benzamide,
[171] N-(4-dimethylaminobenzo[d]isoxazol-3-yl)-3-nitrobenzamide,
[172] adamantane-1-(4-dimethylaminobenzo[d]isoxazol-3-yl)carboxamide,
[173] benzo[b]thiophene-2-(4-dimethylaminobenzo[d]isoxazol-3-yl)carboxamide,
[174] N-(4-dimethylaminobenzo[d]isoxazol-3-yl)-3,3-dimethylbutyramide,
[176] N-(6-amino-4,5,7-trifluorobenzo[d]isoxazol-3-yl)-2,6-difluorobenzamide; and
[177] N-(6-amino-4,5,7-trifluorobenzo[d]isoxazol-3-yl)-2,4-difluorobenzamide;
or a salt thereof.

67. A pharmaceutical composition comprising at least one pharmaceutically acceptable carrier or excipient and a compound according to claim 53.

68. A process for preparing a substituted N-benzo[d]isoxazol-3-ylamine compound according to claim 53, said process comprising:
reacting a compound corresponding to formula IIa 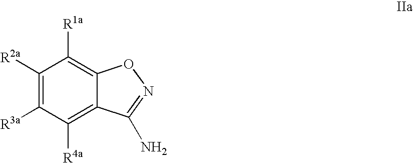
wherein R^{1a} to R^{4a} have the meanings given in claim 53,
(a) in a reaction medium and optionally in the presence of a base,
with a compound corresponding to the formula
R^{5a}—C(═O)—X
wherein
R^{5a} has the meaning given in claim 53, and
X is a leaving group
or
(b) in a reaction medium in the presence of a coupling reagent, and optionally in the presence of a base,
with a compound corresponding to the formula
R^{5a}—C(═O)—OH
wherein R^{5a} has the meaning given above,
to obtain a compound corresponding to formula Ia 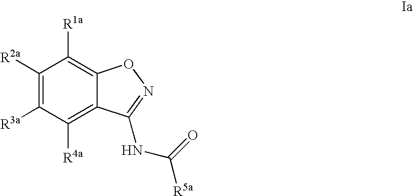
wherein R^{1a} to R^{5a} have the meanings given in claim 53,
and optionally isolating or purifying the obtained compound.

69. A method of treating or inhibiting a disorder or disease state selected from the group consisting of pain, eating disorders, migraine; chronic paroxysmal hemicrania, depression, urinary incontinence, cough, asthma, glaucoma, tinnitus, inflammation, neurodegenerative disorders, cognitive dysfunctions or deficiencies, epilepsy, narcolepsy, diarrhea, gastritis, gastric ulcer, pruritus, anxiety, panic attacks, schizophrenia, cerebral ischemia, muscle spasms, cramps, gastroesaphageal reflux syndrome; alcohol, drug or medicament abuse or dependency, or for inhibiting development of tolerance to medicaments or drugs, regulating food intake, modulating motor activity, regulating the cardiovascular system, local anesthesia, increasing vigilance, increasing libido, diuresis, or antinatriuresis in a subject in need thereof, said method comprising administering to said subject a pharmacologically effective amount of a compound according to claim 53.

70. A method according to claim 69, wherein said disorder or disease state is pain.

71. A method according to claim 70, wherein said pain is selected from the group consisting of acute pain, chronic pain, visceral pain and neuropathic pain.

72. A method according to claim 69, wherein said disorder of disease state is an eating disorder selected from the group consisting of bulimia, anorexia, obesity and cachexia; a neurodegenerative disorders selected from the group consisting of Parkinson's disease, Huntington's disease, Alzheimer's disease and multiple sclerosis; memory impairment; attention deficit syndrome (ADS); nicotine dependency; cocaine abuse or dependency; withdrawal symptoms associated with alcohol, drug or medicament abuse or dependency; or development of tolerance to opioids.
